(19) United States
(12) Patent Application Publication
(10) Pub. No.: US 2002/0051998 A1
(43) Pub. Date: May 2, 2002

(54) Directed evolution of biosynthetic and biodegradation pathways

(75) Inventors: Claudia Schmidt-Dannert, Shoreview, MN (US); Frances H. Arnold, Pasadena, CA (US)

Correspondence Address: DARBY & DARBY P.C., 805 Third Avenue, New York, NY, 10022, US

(73) Assignee: CALIFORNIA INSTITUTE OF TECHNOLOGY

(21) Appl. No.: 09/733,759

(22) Filed: Dec. 8, 2000

Related U.S. Application Data
(60) Provisional application No. 60/169,594, filed on Dec. 8, 1999.
(60) Provisional application No. 60/211,894, filed on Jun. 14, 2000.

(51) Int. Cl. G01N 33/53; C12P 23/00; C12N 5/04; C12N 5/06

(52) U.S. Cl. 435/7.1; 435/325; 435/410; 435/67

(57) ABSTRACT

The present invention relates to engineering new biosynthetic pathways into microorganisms, in particular biosynthetic carotenoid pathways. New and improved catalytic functions of metabolic pathways are created by, for example, site-specific mutation or gene shuffling techniques, to provide for efficient biosynthesis of carotenoids. By applying the described directed evolution techniques, almost any carotenoid could be produced, in a host cell, from one or a few sets of genes. In addition, the described techniques are useful for creating gene or protein libraries for new and uncharacterized carotenoids.

### FIELD OF THE INVENTION

The present invention relates to engineering new biosynthetic or biodegradation pathways into microorganisms, and particularly to using principles of molecular genetic breeding, including mixing genes and creating new catalytic functions by DNA shuffling and in vitro evolution, to create new metabolic pathways.

### BACKGROUND OF THE INVENTION

Natural products cover an enormous diversity of chemical structures and biological functions. However rich this pool of natural structures, it is but a tiny fraction of the structures that could be made biologically—this essentially infinite bank of possible functional molecules is an irresistible target for biological design. Furthermore, many known biologically-active compounds are only found in trace quantities in their natural sources and are difficult or impossible to synthesize chemically. Driving the field of metabolic engineering is the hope that recombinant cells can serve as biosynthetic factories, and possibly even as sources of new molecular diversity (Bailey, J. E., Nature Biotech, 1999;17:616-618; Reynolds, K. A., Proc. Nat'l. Acad. Sci. USA, 1998;95:12744-12746; Cane, et al., Biochemistry, 1999;38:1643-1651; and, Lau, et al., Nature, 1994;370:389-391).

One strategy to create new and improved compounds synthesized in biological systems, e.g., in host such as bacteria, yeast, fungi, algae, and plants, is to alter one or more functions of enzymes involved in the biosynthetic pathway of a compound. However, modifying an enzymatic pathway by rational protein design requires extensive knowledge of structure-function relationships of the enzymes of the pathway, which makes this option unrealistic.

Combinatorial biosynthesis is becoming a key expression in biotechnology and biochemistry, but only a very limited number of examples exist. The power of combinatorial biosynthesis has, for instance, been demonstrated for the synthesis of novel polyketides. Here, mixing and matching of the modular components of polyketide synthases (PKS) have led to the production of novel polyketides and to new mechanistic insights into their structure and function (Carrera and Santi, Currr. Opin. Biotechnol., 1998;9:403-411; Koshla, et al., Biotechnol. Bioeng., 1996;52:122-128; Xue and Sherman, Nature2000;403:571-575, Tanget al., Science 2000;287:640-642).

Unfortunately, biosynthesis of polyketides represents a rather special example of a biosynthetic pathway. Metabolic pathways are usually composed out of several enzymes, catalyzing completely different reactions in contrast to the repeated condensations between carboxylic acid derivatives catalyzed by the PKS modules. Thus, as opposed to polyketide biosynthesis, creation of organic molecule diversity usually requires changing enzyme functions involved in metabolic pathways and/or mixing and matching of enzymes from different origins in a tailor-made pathway. Furthermore, the combinatorial methods applied in polyketide biosynthesis so far are limited to moderate alterations of the PKS complex, involving empirical gene fusion approaches such as domain interaction, substitutions or additions, to create hybrid polyketides, not the addition of new functions foreign to this pathway.

Apart from novel biosynthetic pathways, an important application for metabolic engineering is to explore and improve biodegradation pathways. Biotechnological processes to destroy toxic wastes are particularly challenged by problems such as mixtures of waste compounds, too high or too low concentrations, inhibitory or toxic compounds, bioavailability and biodegradation rate. For instance, aromatic compounds carrying different chemical substituents represent an important class of xenobiotics. The substituents are of ten responsible for the low biodegradability of these compounds. Nevertheless, microbial communities exposed to xenobiotic compounds can of ten adapt to these chemicals, and microorganisms that metabolize them incompletely or completely have been isolated. However, depending on the aromatic xenobiotic and the enzyme composition of catabolic pathways of a certain microorganism, degradation can be either very slow or can lead to the accumulation of intermediates that are not further metabolized and which can be more toxic than the original xenobiotic. This is especially true for many nitro- and chloroaromatic compounds (Pieper, D. H., et al., Naturwissenschaften 1996;83:201-213, Fetzner, S., Appl. Microbiol. Biotechnol. 1998;50:633-657). Metabolic engineering approaches to the design of strains with novel biodegradation capabilities have mainly been based on the combination of pathway modules from different strains, thus creating hybrid pathways (Lee, J-Y, et al., Appl. Environ. Microbiol. 1995 ;61:2211-2217, Panke, S., et al., Appl. Environ. Microbiol. 1998 ;64:748-75 1, Reineke, W. Ann. Rev. Microbiol. 1998;52:287-331, Timmis, K. N., et al., Steffan, R. J. and Untermann, R., Annu Rev Microbiol. 1994;48:525-557). This has led to additional biodegradation abilities of those designed microorganisms. Improvements of catalyst quality and performance needed for effective biodegradation processes, however, are rarely achieved.

Directed evolution has become a powerful tool for the alteration of enzyme functions over the last few years (Kuchner and Arnold, TIBtech. 1997;15:523). Typically, evolutionary processes are mimicked in a test tube by random mutagenesis and/or DNA-shuffling of genes in combination with an efficient screening of the created library. This technique has led, in a relative short time, to the generation of novel enzyme variants with optimized properties for biotechnological applications. For example a p-nitrobenzyl esterase was evolved by four generations of random mutagenesis and two rounds of recombination to yield an enzyme 150-fold more active (in 15-20% DMF) than the wildtype protein (Moore and Arnold, Nat. Biotechnol., 1996; 14:458 and Moore et al., J. Mol. Biol., 1997;272:336). DNA shuffling of a family of cephalosphorinase genes led to a 540 fold increase of moxalactamase activity (Cramer et al., Nature, 1998;391:288). However, it has not been shown that genes with the required synthesis or degradation potential can be selected from nature, adapted and assembled into new pathways for biological products used in medicine or agriculture.

Thus, there is a need in the art for strategies to recreate pathways in recombinant hosts to optimize the production of useful compounds. This is particularly true for complex chemical compounds requiring multi-step synthesis, suffering from low yields and, accordingly, low availability and/or high prices. There is a further need for new structures having improved and/or novel qualities over the original compounds, requiring the development of new pathways for their synthesis. Especially, libraries of synthetic pathways could provide a wide range of compounds never before synthesized in a particular host, or at all. There is also a need in the art for new and improved biodegradation pathways, either to produce metabolites of interest or degrading waste products. The present invention addresses these and other needs in the art.

### SUMMARY OF THE INVENTION

The present invention provides recombinant systems created by directed evolution that provide for efficient biosynthesis or biodegradation of a variety of compounds. Thus, in one aspect, the invention provides a host cell, as well as a library of host cells, comprising one or more expression vectors that express one or more mutated genes encoding a biosynthetic enzyme operably with an expression control sequence, which host cell or host cells produce(s) the compound or compounds of interest. Accordingly, one or a few sets of genes could be used to make almost any variant within a selected class of compounds. Preferably, the compound is an uncharacterized compound. Alternatively, the compound is not endogenously produced by the host cell, or, more preferably, that type of host cell.

In particular, a preferred feature of the invention is the discovery that genes from unrelated metabolic pathways from the same or from different organisms can be modified by molecular evolution to yield a new gene. Thus, in one aspect the mutated gene is a combination of genes from different metabolic pathways. In another aspect, directed evolution of the invention permits introduction of a mutated gene into a host cell to produce an enzyme that functions in an unrelated or different metabolic pathway.

The invention specifically provides for directed evolution of carotenoid biosynthetic pathways.

The invention further provides a nucleic acid encoding for an biosynthetic or biodegradation enzyme modified according to the invention. Also provided is an expression vector comprising the nucleic acid operably associated with an expression control sequence, and a host cell comprising the expression vector.

The invention further provides a method for producing a compound. The method comprises culturing a host cell of the invention under conditions that permit production of the compound by the host cell. In particular, this method permits the production of compounds in microorganisms that do not endogenously produce them, and permits the production of new compounds.

The invention further provides a method for creating a new biosynthetic pathway. This method comprises detecting production of a selected compound in a host cell modified by transduction with a mutated gene encoding a biosynthetic enzyme involved in the pathway producing the compound, wherein the compound is not produced by an unmodified host cell.

The invention also provides optimization of biosynthetic pathways by directed evolution.

In addition, the invention provides a method for creating a new biodegradation pathway, and for optimization of a biodegradation pathway by directed evolution.

Moreover, the invention provides for the creation of new biometabolic pathways by subjecting a combination of different and/or unrelated biometabolic pathways to directed evolution according to the invention.

Further, the invention provides for the optimization of a biosynthetic or biodegradation pathway by combining enzymes from different organisms and/or different pathways, and modifying the resulting new pathway by directed evolution.

The invention also provides for gene libraries encoding for novel pathways created according to the methods described herein. Furthermore, the invention provides for libraries of novel pathways, created according to the invention.

The invention also provides for screening techniques, to enable identification and selection of an enzymatic pathway leading to a novel and/or improved compound.

Additionally, the invention provides for an enzyme which has been modified by directed evolution, and which functions in a biochemical pathway in a host cell.

### DESCRIPTION OF THE DRAWINGS

FIG. 1. C₄₀ carotenoid biosynthesis branches into a variety of pathways to acyclic and cyclic carotenoids, for which biosynthetic genes from bacteria have been cloned (for a review, see, Hirschberg, In: Carotenoids: Biosynthesis and Metabolism, Vol. 3, Carotenoids, G. Britton, Ed., Basel: Birkhäuser Verlag, 1998, pp.148-194; and Britton, G., Id., pp.13-147). Dotted arrows indicate how the central desaturation pathway has been extended to obtain the fully conjugated 3,4,3,4-tetradehydrolycopene and subsequent branching of this pathway for the synthesis of torulene.

FIGS. 2A, 2B, and 2C. (Color drawings) (A) HPLC analysis of carotenoid extracts of E. coli transformants carrying plasmids pAC-crtE_{EU}-crtB_{EU} and pUC-crtI_{EU} expressing the wildtype phytoene desaturase. (B) Recorded absorption spectra of individual HPLC peaks. (C) The corresponding carotenoid extract (orange) is shown. Results of pUC-crtI_{EH} were similar to pUC-crtI_{EU}.

FIGS. 3A, 3B, and 3C. (Color drawings) (A) HPLC analysis of carotenoid extracts of E. coli transformants carrying plasmids pAC-crtE_{EU}-crtB_{EU} and pUC-I14 expressing desaturase mutant I14. (B) Recorded absorption spectra of individual HPLC peaks. (C) The corresponding carotenoid extract (pink) is shown.

FIGS. 4A, 4B, and 4C. (Color drawings) (A) HPLC analysis of carotenoid extracts of E. coli transformants carrying plasmids pAC-crtE_{EU}-crtB_{EU} and pUC-I25 expressing desaturase mutant I25. (B) Recorded absorption spectra of individual HPLC peaks. (C) The corresponding carotenoid extract (yellow) is shown.

FIGS. 5A and 5B. (Color drawings) Cell pellets of E. coli transformants expressing wildtype and mutant cyclases. (A) JM109 carrying plasmid pUC-crtY_{EU} or pUC-Y2, together with pAC-crtE_{EU}-crtB_{EU} -crtI_{EU} or pAC-crtE_{EU}-crtB_{EU}-I14. (B) JM 109 transformants carrying pAC-crtE_{EU}-crtB_{EU}-I14 and various cyclase mutants.

FIGS. 6A and 6B. (A) HPLC analysis of carotenoid extract of E. coli transformant carrying the plasmids pAC-crtE_{EU}-crtB_{EU} -I14 and pUC-crtY_{EU} expressing desaturase mutant I14 together with wildtype lycopene cyclase. Double peaks indicate different geometrical isomers. Peak 1:β, β-carotene (λₘₐₓnm: 425 450 478). (B) Recorded absorption spectra of individual peaks. Results for crtY_{EH} were similar to crtY_{EU}.

FIGS. 7A and 7B. (A) HPLC analysis of carotenoid extract of E. coli transformant carrying the plasmids pAC-crtE_{EU}-crtB_{EU}-crt I_{EU} and pUC-crtY_{EU} expressing wildtype phytoene desaturase together with wildtype lycopene cyclase. Double peaks indicate different geometrical isomers. Peak 1:β, β-carotene (λₘₐₓnm: 425 450 478), peak 2: β-zeacarotene (λₘₐₓnm: 406 428 454). (B) Recorded absorption spectra of individual peaks. Results for crtY_{EH} were similar to crtY_{EU}.

FIGS. 8A and 8B. (A) HPLC analysis of carotenoid extract of the E. coli transformant carrying plasmid pAC-crtE_{EU}-crtB_{EU} -I14 and pUC-Y2 expressing desaturase mutant I14 together with cyclase mutant Y2. The following carotenoids were identified: peak 1:3,4,3′,4′-tetradehydrolycopene(λₘₐₓnm:480 510 540, M+at m/e=532.4), peak2: lycopene (λₘₐₓnm: 444 470 502, m+at m/e=536.4), peak 3: torulene (λₘₐₓnm: 454 480 514, M+at m/e=534.5), peak 4: α, Ψ-carotene (αₘₐₓnm: 435 450 478, M+at m/e 536.4), peak 5: β, β-carotene (λₘₐₓnm: 425 450 478, M+at m/e=536.4). Double peaks represent different geometrical isomers. (B) Recorded absorption spectra of individual peaks.

FIGS. 9A and 9B. (A) HPLC analysis of carotenoid extract of the E. coli transformant carrying plasmids pAC-crtE_{EU}-crtB_{EU} -crtI_{EU} and pUC-Y2 expressing wildtype desaturase together with cyclic mutant Y2. Peaks 4 and 5 correspond to the carotenoid peaks identified in FIG. 6. (B) Recorded absorption spectra of individual peaks.

FIG. 10. Pathways for the cleavage of catechol and chlorocatechol.

### DETAILED DESCRIPTION

The present invention advantageously provides for the efficient biosynthesis of compounds, particularly via genetic (molecular) breeding. The invention provides for the production of various compounds in high yield, particularly in organisms that normally do not produce such compounds, but also in microorganisms that can produce the compounds, in which case the production can be rendered more efficient.

In one embodiment of the invention, carotenoids can be produced in bacteria, which normally do not produce carotenoids. In another embodiment, carotenoid production in microorganisms such as, but not limited to, yeasts, molds, fungi, and algae, can be improved, e.g., by overcoming problems related to endogenous precursor and metabolite capacities.

The Examples presented herein describe the application of the invention for directed evolution of carotenoid biosynthetic pathways, providing compelling evidence for an equally successful application of the invention for both related and unrelated biosynthetic pathways. For instance, a nucleic acid encoding a novel phytoene desaturase (crtI) is provided for the production of novel and/or modified carotenoids. The specific mutants include an E. uredovora crtI comprising an arginine to histidine modification at position 332 and a glycine to serine substitution at position 470, and an E. uredovora/E. herbicola hybride-crtI comprising a proline to lysine modification at position 3, a threonine to valine modification at position 5, a valine to threonine modification at position 27, and a leucine to valine modification at position 28. In another specific embodiment, a nucleic acid encoding a lycopene cyclase is provided. In particular, a lycopene cyclase (crtY) from E. uredovora comprises an arginine to histidine modification at position 330 and a proline to serine modification at position 367. The invention also provides an expression vector comprising this nucleic acid operably associated with an expression control sequence, and a host cell comprising the expression vector.

Similarly, novel or improved non-ribosomal peptide synthesis pathways can be created according to the invention. The invention further contemplates using directed evolution techniques to engineer plant cells and plants to produce desired compounds. Moreover, it has been discovered that these systems can produce compounds never before produced by the microorganism or plant, and, indeed, novel compounds, including novel carotenoids, tetrapyrroles, polyketides, flavonoids, terpenoids, aminoglycosides that have not been characterized to date. As one of ordinary skill can readily appreciate, the ability of the directed evolution technique of this invention to produce known or characterized compounds, or unknown or uncharacterized compounds, provides a powerful tool for developing products from important classes of molecules. This invention overcomes the inability of naturally existing biosynthetic or chemical synthetic pathways to create a multitude of compounds of interest within a reasonable time-frame, much less a multitude of derivatives of each possible compound.

In addition, the invention provides for the biodegradation of compounds, in particular aromatic compounds. The invention further contemplates improving the efficiency of chosen biodegradation pathways to increase degradation rate of potentially toxic, as well as non-toxic, compounds. The biodegradation pathways to be modified according to the invention are preferably, but not limited to, naturally occurring pathways in chosen microorganisms, plants, or other useful hosts. In the context of the invention, an altered or improved biodegradation pathway can also be used in the production of novel or improved compounds which are metabolites of other compounds.

Creation of tailor-made biosynthetic or metabolic pathways provides a superior tool for the production or degradation of novel compounds, which can either not be chemically synthesized or degraded at all, or only in very limited yields. Additionally, biochemical characterization of the enzymes and enzyme variants involved in these pathways will lead to new information on their function.

The term “directed evolution” refers to a combination of metabolic engineering with molecular evolution of new catalytic functions, to discover new pathways to synthesize or metabolize existing and/or new compounds. Directed evolution can also be referred to as Molecular Breeding™. The principles of directed evolution, including in vitro directed evolution, can access this diversity. Directed evolution involves mixing wild-type alleles from different parents and spontaneous mutation of alleles, or combinations of both, followed by a selection for the desired properties. Mixing and matching genes from different sources to create new biosynthetic functions, e.g., by DNA shuffling, random mutagenesis, recombination and selection (Stemmer, Nature, 1994;370:389; Crameri et al., Nature, 1998;391 :288, Joo et al., Nature, 1999;399:670; Arnold and Volkov, Curr. Op. Chem. Biol., 1999;3:54), all in the absence of detailed information on enzyme structure or catalytic mechanism, and metabolic engineering expression of these genes, establishes new biosynthetic or biometabolic pathways. Preferably, directed evolution involves creating new metabolic pathways by combining gene products from different or unrelated pathways, preferably modified by molecular evolution.

“Molecular evolution” involves modifying target genes, e.g., by random or site-specific mutagenesis, gene shuffling, or other mutagenic techniques, to yield a “mutant gene encoding a biometabolic enzyme”. When incorporated in a metabolically engineered pathway, selection of mutants proceeds by identifying a desired phenotype, e.g., color of the compound in question.

The term “biometabolic” herein includes both “bioanabolic”, i.e., biosynthetic, and “biocatabolic”, i.e., biodegradation.

“Metabolic engineering” involves rational pathway design and assembly of bioanabolic/biosynthetic or biocatabolic/biodegradation genes and control elements, with optimization of metabolic flux by regulation and optimization of transcription, translation, protein stability and protein functionality using genetic engineering and appropriate culture condition. The bioanabolic or biocatabolic genes are heterologous to the host (e.g., microorganism or plant), either by virtue of being foreign to the host, or being modified by mutagenesis, recombination, and/or association with a heterologous expression control sequence in an endogenous host cell. Appropriate culture conditions are conditions of culture medium pH, ionic strength, nutritive content, etc.; temperature; oxygen/CO₂/nitrogen content; humidity; and other culture conditions that permit production of the compound by the host cell, i.e., by the metabolic action of the cell. Appropriate culture conditions are well known for microorganisms that can serve as host cells.

The term a “new catalytic function” refers to a catalytic functions mediated by a mutated biosynthesis or biodegradation enzyme and the compound it produces. A “new (or novel) compound”, also termed “heterologous compound” herein, refers to a compound not found in the organism from which the wild-type biosynthetic gene was originally isolated (the natural source) or, if found in such an organism, has not been characterized, or represents a minor component of an endogenous synthetic or degradation pathway (e.g., less than about 5%, more preferably less than about 1%, and more preferably still less than about 0.1% of total synthesis or production of the compound). Further, catalytic function does not only involve the product and substrate specificity and the catalyzed chemical reaction, but also the activity and stability of the pathway and/or an enzyme of the pathway, which can change the flux in a pathway.

### Definitions

In a specific embodiment, the term “about” or “approximately” means within 20%, preferably within 10%, and more preferably within 5% of a given value or range. Alternatively, especially in biological systems, the term “about” means within about a log (i.e., an order of magnitude), preferably within a factor of two of a given value, depending on how quantitative the measurement.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably selectively multiplied using PCR, or excised, from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified. An isolated metabolite includes a cellular extract containing the metabolite.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, including thin-layer chromatography (TLC); gel electrophoresis, immunoassays, composition analysis, biological assays, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, high-performance liquid chromatography (HPLC), reversed-phase (RP) HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Compounds can be purified by chromatography, particularly by RP-HPLC. Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

The use of italics with reference to a specific biosynthesis or biodegradation enzyme indicates a nucleic acid molecule (e.g., cDNA, gene, etc.); normal text indicates the polypeptide or protein.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

The term “chimeric” or “chimera” herein refers to a polynucleotide, gene, polypeptide, protein, or metabolic pathway which comprises parts derived from different species, different metabolic pathways, or both.

As used herein, the term “homologous” in all its grammatical forms and spelling variations refers to the relationship between proteins that possess a “common evolutionary origin, ” including proteins from superfamilies (e.g., the immunoglobulin superfamily) and homologous proteins from different species (e.g., myosinlight chain, etc.) (Reecket al., Cell 50:667, 1987). Such proteins (and their encoding genes) have sequence homology, as reflected by their sequence similarity, whether in terms of percent similarity or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous, ” when modified with an adverb such as “highly, ” may refer to sequence similarity and may or may not relate to a common evolutionary origin.

In a specific embodiment, two DNA sequences are “substantially homologous” or “substantially similar” when the encoded polypeptides are at least 35-40% similar as determined by one of the algorithms disclosed herein, preferably at least about 60%, and most preferably at least about 90 or 95%) in a highly conserved domain, or, for alleles, across the entire amino acid sequence. Sequence comparison algorithms include BLAST (BLAST P, BLAST N, BLAST X), FASTA, DNA Strider, the GCG (Genetics Computer Group, Program Manual for the GCG Package, Version 7, Madison, Wis.) pileup program, etc. using the default parameters provided with these algorithms. Sequences that are substantially homologous can be identified by comparing the sequences using standard software available in sequence data banks, or in a Southern hybridization experiment under, for example, stringent conditions as defined for that particular system.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

“Amplification” of DNA as used herein denotes the use of polymerase chain reaction (PCR) to increase the concentration of a particular DNA sequence within a mixture of DNA sequences. For a description of PCR see Saiki et al., Science, 239:487, 1988.

A “nucleic acid molecule” refers to the phosphate ester polymeric form of ribonucleosides (adenosine, guanosine, uridine or cytidine; “RNA molecules”); or deoxyribonucleosides (deoxyadenosine, deoxyguanosine, deoxythymidine, or deoxycytidine; “DNA molecules”); or any phosphoester analogs thereof, such as phosphorothioates and thioesters, in either single stranded form, or a double-stranded helix; or “protein nucleic acids” (PNA) formed by conjugating bases to an amino acid backbone; or nucleic acids containing modified bases, for example thiouracil, thio-guanine and fluoro-uracil. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. This includes single- and double-stranded molecules, i.e., DNA-DNA, DNA-RNA and RNA-RNA hybrids. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “coding sequence” or a sequence “encoding” an expression product, such as a RNA, polypeptide, protein, or enzyme, is a minimum nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG, though as shown herein, alternative start codons can be used) and a stop codon. The coding sequence may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′ -non-coding regions, and the like.

The term “gene”, also called a “structural gene” means a DNA sequence that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” or “operably (or operatively) associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as mRNA or a protein. The expression product itself, e.g. the resulting mRNA or protein, may also be said to be “expressed” by the cell.

The term “transfection” means the introduction of a heterologous nucleic acid into a cell. The terms “transduction” and “transformation” as used herein mean the introduction of a heterologous gene, DNA or RNA sequence to a host cell, so that the host cell will express the introduced gene or sequence to produce a desired product. The introduced gene or sequence may also be called a “cloned” or “heterologous” gene or sequence, and may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which heterologous DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector of ten contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will of ten include one or more replication systems for cloning or expression, one or more markers for selection in the host, e.g. antibiotic resistance, and one or more expression cassettes.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Preferably, a host cells of the invention is transformed with one or more genes encoding a biosynthetic or biodegradation function. Another example of a host cell is a cell that accumulates or secretes compound of interest. Host cells can further be used for screening or other assays, as described infra. Host cells can be cultured cells in vitro or one or more cells in a plant, e.g., a transgenic plant or a transiently transfected plant. Host cells of the invention include, though they are not limited to, bacterial cells (e.g., E. coli, Synechocystis sp., Z. mobilis, Agrobacterium tumefaciens, and Rhodobacter); yeast cells (e.g., S. cerevisiae, Candida utilis, Phaffia rhodozyma ); fungi (e.g., Phycomyces blakesleeanus); algae (e.g., H. pluvalis); and plants (e.g., Arabidopsis thaliana).

A “microorganism” as used herein refers to a bacteria, yeast, mold, fungus, or algae, and also, for the purposes of this invention, or plant cell. Microorganisms are modified by directed evolution to produce a heterologous compound. They can also be the source of biosynthetic genes.

The term “expression system” means a host cell and compatible vector under suitable conditions, e.g. for the expression of a protein coded for by foreign DNA carried by the vector and introduced to the host cell. An expression system of the invention provides a biosynthetic or biodegradation pathway.

The term “heterologous” as used herein refers to a combination of elements not naturally occurring in a given host cell. For example, heterologous DNA refers to DNA not naturally located in the cell, or in a chromosomal site of the cell. A heterologous gene is a gene in which the regulatory control sequences are not found naturally in association with the coding sequence. A heterologous compound is a compound that is not normally produced in the host cell which has been subjected to directed evolution in accordance with the invention to produce such a compound.

A mutated gene encoding a biosynthesis or biodegradation enzyme can be inserted into an appropriate cloning vector. A large number of vector-host systems known in the art may be used. Possible vectors include, but are not limited to, plasmids or modified viruses, but the vector system should be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2 μ plasmid. The necessary transcriptional and translational signals can be provided on a recombinant expression vector.

Expression of a mutated biosynthesis or biodegradation enzyme may be controlled by any promoter/enhancer element known in the art, but these regulatory elements should be functional in the host selected for expression, including prokaryotic expression vectors such as the β-lactamase promoter (Villa-Komaroff, et al., Proc. Natl. Acad. Sci. USA, 1978, 75:3727-3731), or the tac promoter (DeBoer, et al, Proc. Natl. Acad. Sci. USA, 1983, 80:21-25); see also “Useful proteins from recombinant bacteria” in Scientific American, 1980, 242:74-94; and promoter elements from yeast or other fungi such as the Gal 4 promoter, the ADC (alcohol dehydrogenase) promoter, PGK (phosphoglycerol kinase) promoter, and alkaline phosphatase promoter.

### Directed Evolution of Enzymatic Pathways

### General

The invention provides for the creation of novel enzymatic biosynthetic or biodegradation enzymes by directed evolution techniques, preferably within the context of an assembled pathway. Methods used for directed evolution according to the invention preferably include gene shuffling, error-prone PCR, or random mutagenesis, depending on the gene to be evolved. While these techniques have been used to modify, or “fine tune”, an existing enzymatic function, placed in the context of techniques for directed evolution of whole pathways, new functions or traits not previously exhibited by a particular enzyme, or any existing enzymatic pathway, may be introduced.

In one embodiment, selected enzymes from at least two pathways from different hosts are combined. Preferably, the combined pathway is subjected to directed evolution to optimize, adapt, and/or attune the resulting pathway to, e.g., create compounds displaying novel and/or useful features. If desired, at least one of the biosynthetic or biodegradation enzymes in one pathway has been subjected to directed evolution prior to combining the selected enzymes. The biosynthetic or biodegradation enzymes to be combined and subjected to directed evolution according to the invention may be anything from closely related to completely unrelated. Alternatively, the pathways may be related, but taken from different organisms. In addition, pathways may be substantially unrelated and taken from different hosts.

In another embodiment, variant enzyme libraries, preferably within the context of an assembled pathway, can be created by co-transformation with two plasmids that are stably propagated together as follows; Genes producing the precursors serving as substrates for the target enzyme(s) are cloned into one plasmid, and genes for the enzymes subjected to in vitro evolution are cloned into another plasmid, together with suitable sequences for regulating expression (via, e.g., promoter or operon control). Different biosynthetic genes are evolved by random mutagenesis and/or gene shuffling and introduced to the pathway. Enzyme variants leading to the production of novel compounds, or more efficient synthesis/catabolism of known compounds, can be combined in a modular way, resulting in additional novel pathways. Also, modular vectors can be constructed to allow for the expression of several biosynthetic genes.

Optimization of microbial production levels in general can be achieved by:

optimizing protein expression levels for maximal production by in vitro mutagenesis of target genes and/or classical regulation of gene expression;

synthesizing the compound of interest by directed evolution of the selected enzymes;

implementing the evolved pathways in microbial hosts;

biochemical characterization of novel enzyme variants and pathways.

In yet another embodiment, production of a compound in a microorganism which does not naturally synthesize this particular compound may be accomplished by, e.g., extending a related pathway with genes encoding for appropriate enzymes taken from other organisms. Also, once a biosynthetic pathway is created in one microorganism the pathway can be transferred to a different host system.

Bioanabolic or biocatabolic genes needed for application of the invention can be cloned, e.g., by retro-PCR from genomic DNA of the respective microorganism. Microorganisms can be obtained from, e.g., the German Culture Collection (DSM), the American Type Culture Collection (ATCC), other depositories, or naturally occurring bacteria.

### Screening Techniques

Efficient screening techniques are needed to enable selection of pathways for directed evolution. Preferably, suitable screening techniques for compounds produced by the enzymatic pathways allow for a rapid and sensitive screen for the properties of interest. Visual (calorimetric) assays are optimal in this regard, and are easily applied for compounds with suitable light absorption properties. Moreover, the successes of combinatorial chemistry in drug development and directed enzyme evolution have spurred the development of more and more sophisticated screening technology. This includes, for instance, high-throughput HPLC-MS analysis, where screening robots are connected to HPLC-MS systems for automated injection and rapid sample analysis. These techniques allow for high-throughput detection and quantification of virtually any desired compound. HPLC-MS, TLC, and screening of microtiter plates using a plate reader, can be used to identify novel carotenoids demonstrating only small differences in their absorption properties. Screening and selection techniques for directed enzyme evolution, which techniques may be adaptable for use in the invention, have recently been reviewed (Zhao, H. and Arnold, F., Curr. Opin. Struct. Biol. 1997; 7: 480-485; Hilvert, D. and Kast, P., Curr. Opin. Struct. Biol. 1997; 7: 470-479).

### Terpenoids

### General

Terpenoids constitute the largest family and chemically most diversified group of natural products. An amazing number of 23,000 different terpenoid compounds have been described and hundreds of new structures continue to be identified every year (Connolly & Hill, Dictionary of Terpenoids, Chapman & Hall, London, 1991). The enormous diversity of terpenoid structures reflects the importance and the diversity of functions of terpenoids in biological systems. Terpenoids serve as hormones (e.g. gibberellins), photosynthetic pigments (phytol, carotenoids), antioxidants (e.g. carotenoids), electron carrier (e.g. ubiquinone), mediators of polysaccharide assembly (polyprenyl diphosphates) and as membrane components (sterols, hopanoids). Monoterpenes are common fragrances and flavors. Many sesquiterpenes and diterpenes function as defensive agents, visual pigments, antitumor drugs and as signal transduction components. In plants, the monoterpenoids (10 carbon backbone) are known as constituents of essential oils and are responsible for the characteristic scent of the plants in which they occur, and a diversity of structural types are used as flavorings and scents. In addition, many of these compounds have biological activity, and many of the therapeutically active components in plants and herbs that have been traditionally used for the treatment of a variety of diseases are terpenoids. Examples include artemisinin, a sesquiterpene isolated from wormwood that is used for the treatment of fevers and malaria; taxol, a diterpene isolated from pacific yew that is one of the most effective anticancer drugs and forskolin, a diterpene isolated from an Indian medicinal plant lowers blood pressure and has cardio active properties. A variety of terpenoids have antibacterial and antifungal properties or are potent cell toxins like for example the trichoethecene sesquiterpenes isolated from certain fungi. Important terpenoid agrochemicals are e.g. the insecticidal pyerethrins (monoterpenes) and azadrachtin (triterpenoid). For a review on medicinal and agrochemical properties of terpenoids, see Dewick (Medicinal Natural Products, John Wiley & Sons, New York, 1998). Both the amazing chemical diversity and functional diversity of terpenoids, makes them possible the most promising class of natural products for the discovery of a variety of compounds of economic value (Sacchettini and Poulter, Science 1997;277:1788-1790).

Various enzymatic pathways leads to the formation of a variety of terpenoids, e.g., monoterpenoids, sesquiterpendoids (15 carbon backbone), diterpenoids (20 carbon backbone), and tetraterpenoids (40 carbon backbone). Of the monoterpenes, there are three main groups; acyclic terpenes such as geraniol, moncyclic species such terpineol, and bicyclic species such as camphor and thujone.

### Terpenoid Biosynthetic Genes

The biosynthetic pathways for terpenes, carotenoids, and steroids all arise all begin with the condensation of two molecules of acetyl-CoA, catalyzed by the enzyme acetoacetyl-CoA thiolase. The second step is catalyzed by the enzyme hydroxyglutaryl-SCoA (HGM-SCoA) synthase. The product, HMG-CoA. is reduced to produce mevalonic acid by HMG-CoA reductase. The mevalonic acid is phosphorylated to produce MVA-5 pyrophosphate, which is carboxylated to produce isopentenyl pyrophosphate (IPP). In the first committed step in isoprenoid biosynthesis, the linear 10-carbon (C10) geranyl diphosphate (GDP) molecule is formed via a head-to-tail condensation (1′-4 addition) of two C5 isoprene units; IPP and its isomer; dimethylallyl diphosphate (DMAPP). GDP, the precursor of all terpenoids; geranyl diphosphate, may thereafter undergo chain elongation and/or cyclization.

Chain Elongation

Head-to-tail additions of C5 isoprene units (IPP) lengthen the polyprenyl chain to produce the linear C15 sesquiterpene farnesyl diphosphate (FDP), the C20 diterpene geranylgeranyl diphosphate (GGDP), and so on. These sequential condensations of polyprenyl diphosphates are catalyzed by chain-length selective prenyl transferases. A number of prenyl transferases catalyzing the synthesis of polyprenyl chains containing up to ten C5 isoprene units (e.g., deacaprenyl diphosphate synthase from S. pombe) have been cloned from plants and microorganisms. By contrast, squalene (C30) and phytoene (C40) are produced by a head-to-head condensation of two building blocks FDP (C15) or GGDP (C20), respectively. During this type of condensation, no reactive diphosphate ester is retained in the final product.

Cyclization

Linear mono-, sesqui- and diterpenes may be transformed into a great variety of cyclic compounds reactions, catalyzed by terpene cyclases and using a cationic mechanism that involves initial carbocation formation. A different class of cyclases transforms oxidosqualene into sterols and squalene into hopanoids. Cyclic carotenoids are synthesized from phytoene derivatives by cyclization of only the end-groups. Terpene cyclization can be subdivided into the generation of a reactive carbocation, stepping of the carbocation through the substrate chain (of ten involving de- and reprotonation) to produce a terminal carbocation and quenching of this carbocation by a base. The cyclization reactions are special cases of SN1-like alkylations and thus correspond to the prenyl transferase reactions (Lesburg et al., Curr. Opin. Biotechnol. 1998 8:695-703; Wendt and Schulz, Structure 1998 6:127-133). Depending on the mechanism of how the first reactive carbocation is generated, terpene cyclase can be divided into two general classes: Class I enzymes generate an allylic carbocation by the release of a diphosphate group. This class includes the prenyl transferases, the mono- and sesquiterpene synthases as well as many diterpene cyclases that catalyze the formation of non-aromatic, macrocyclic diterpenes. Common to these enzymes is a DDXXD-sequence motif that binds Mg²⁺ ions, which facilitate diphosphate release. Class II enzyme on the other hand generate the carbocation by protonating a C-C double bond or the corresponding epoxide. This class includes the triterpene (squalene and oxidosqualene cyclases) and carotenoid cyclases as well as some diterpene cyclases catalyzing the synthesis of aromatic diterpene.

Enzyme Structures

Presently, the structures of one prenyl transferase (chicken FDP synthase) and two sesquiterpene cyclases (tobacco 5-epi-asristolochene synthase and Streptomyces pentalenene synthase) belonging to class I have been solved (Tarshis et al., Biochemistry 1994;33:10871; Lesburg et al., Science 1997;277:1870-1824; Starks et al., Science 1997; 277:1815-1820). These enzymes share a fold that consists predominantly of α-helices, five of which surround their deeply buried hydrophobic active sites. This fold has been named terpenoid synthase fold (class I). Except for the common DDXXD motif, no significant sequence homology exists between the three enzymes.

In addition, the structure of one class II enzyme, squalene-hopene cyclase from Alicyclobacillus, is known (Wendt et al., Science 1997;277:1811 -1815). Its structure differs grossly from those of the class I enzymes. The structure of squalene-hopene cyclase consists of two domains: a major regular (aa)6 barrel domain and a minor domain with a similar (aa) barrel fold. The active site is a large cavity located in the middle of the molecule. In contrast to the water-soluble sesquiterpene synthases, squalene-hopene cyclase is an internal membrane protein and a non-polar channel to the non-polar membrane moiety connects its active site to where the substrate squalene is dissolved.

In both classes of terpenoid synthases are the reactive carbocation intermediates shielded in a deeply buried hydrophobic active center. The required substrate conformation necessary for cyclization seems to be enforced mainly by aromatic hydrophobic residues lining the active site cavity.

Terpenoid Synthases

Monoterpene synthases catalyze the conversion of GDP to cyclic monoterpenes with either one or two rings. Because of the C2-C3 trans-double bond in GDP, monoterpene cyclization requires an isomerization step prior to cyclization. GDP is ionized with the assistance of a divalent metal ion that stabilizes the formed allylic carbocation diphosphate anion pair. Following rotation into the cis conformer, cyclization and release of the diphosphate anion results in the formation of the terminal carbocation. From this universal intermediate, the reaction can take one of several routes involving internal additions to double bonds, hydride shifts or rearrangements before the terminal carbocation is quenched by deprotonation or water. All monoterpene cyclases catalyze both the isomerization and cyclization resulting in the synthesis of approximately 1,000 different monoterpene structures from GDP (McCaskill and Croteau, Adv. Biochem. Eng. Biotechnol. 1997;55:107-146; Bohlmann et al., Proc. Natl. Acad. Sci. USA 1998;95:4126-4133).

To date, only about 20 monoterpene cyclases have been cloned. (See Table 1 and Bohlmann et al., 1998, supra). Here, monoterpenes are compounds of the oleoresins where they function as defensive agents and contribute to the fragrance and flavor of plants. Investigation of the substrate and product spectrum of monoterpene synthases showed their ability to produce multiple products (Bohlmann et al., J. Biol. Chem. 1997;272:21784-21792; Wise et al., Proc. Natl. Acad. Sci. USA 1998;273:14891-14899). Although some monoterpene cyclases, like e.g. the (−)-4S limonene cyclase from Mentha, are highly product specific, the majority of the investigated monoterpene cyclases synthesize significant amounts of one to two minor products in addition to the major product. This has been attributed to the high reactivity of the carbocation intermediates that allow proceeding of several reaction routes. However, a cyclase can only be either R or S specific.

Sesquiterpene Synthases

Sesquiterpene synthases convert FDP to over 200 different cyclic skeletons very similar to the reactions catalyzed by monoterpene cyclases. With 15 C-atoms and 3 double bonds in FDP, there are considerably more reaction routes possible compared to GDP. The number of possible sesquiterpene structures existing in nature has been estimated to be more than 7,000 (McCaskill and Croteau, Adv. Biochem. Eng. Biotechnol. 1997;55:107-146; Bohlmann et al., Proc. Natl. Acad. Sci. USA 1998;95:4126-4133). As in the case of the monoterpene cyclases, the reactive carbocation is created by ionization of the diphosphate ester. This cation can attack either the terminal or the internal C6-C7 double bond. Recruitment of the internal double bond in the cyclization reaction requires isomerization of the C2-C3 trans-double to the cis configuration via a NDP (Nerolidyl diphosphate) intermediate. Formation of macrocyclic sesquiterpenes such as germacranes, humulanes or pentalenene requires no isomerization, but synthesis of cyclohexanoic sesquiterpenes like bisabolanes, cadinanes or cedranes proceeds via a NDP intermediate. A variety of rearrangements, hydride shifts and methyl migrations prior to the quenching of the terminal carbocation by deprotonation or water allow the formation of a large number of different sesquiterpene structures.

Approximately 15 sesquiterpene cyclase genes have been isolated from fungi, bacteria and plants. (See Table 1). Similar to the monoterpene synthases, the product specificity of many sesquiterpene cyclases is fairly broad. Especially plant cyclases that are constitutively expressed produce a number of minor sesquiterpene products in addition to the major product. Extreme cases are humulene synthase and selinene synthase, which produce 34 and 53, respectively, different sesquiterpenes (Steele et al., J. Biol. Chem. 1998;273:2078-2089). On the other hand are inducible plant cyclases of ten very product specific as are fungal and bacterial cyclases (Bohlmann et al., Proc. Natl. Acad. Sci USA 1998; 95:6756-6761; Cane et al. Biochemistry 1994;33:5846-5857). These cyclases produce sesquiterpenes that have very specific defensive functions like e.g. pentalenenolactone antibiotics derived pentalenene and the wound protecting terpenes cadinene and bisabolene. Interestingly, most sesquiterpene cyclases accept also GDP as substrate and thus can produce monoterpenes, although at a much lower rate than sesquiterpenes. The C20 prenyl diphosphate GGDP, however, is not cyclized by the sesquiterpene cyclases that have been investigated so far.

TABLE 1
Isolated Terpenoid Synthase Genes

| | | | Group (Publication |
|---|---|---|---|
| Terpene synthase | Source | Comments | Year) |
| Monoterpene synthases | | | |
| (−)-Limonene synthase | Abies grandis | | Croteau (1997) |
| (−)-Myrcene synthase | Abies grandis | | Croteau (1997) |
| (−)-Pinene synthase | Abies grandis | | Croteau (1997) |
| (3R)-Linalool synthases (2) | Artemisia annua | | Chen, Croteau (1999) |
| (+)-cx-Pinene synthase | Salvia officinalis | | Croteau (1998) |
| (+)-Camphene synthase | Salvia officinalis | | Croteau (1998) |
| Bornyl DP synthase | Salvia officinalis | | Croteau (1998) |
| (+)-sabinene synthase | Salvia officinalis | | Croteau (1998) |
| 1,8-cineole synthase | Salvia officinalis | | Croteau (1998) |
| (−)-Camphene synthase | Abies grandis | | Croteau (1999) |
| (−)-βPhellene synthase | Abies grandis | | Croteau (1999) |
| Terpinolene synthase | Abies grandis | | Croteau (1999) |
| (−)-Limonene/(−)-α-Pinene | Abies grandis | | Croteau (1999) |
| synthase | | | |
| 4S-Limonene synthase | Mentha spicata | | Croteau (1993) |
| 4S-limonene synthase | Perilla frutescens | | Croteau (1996) |
| S-Linalool synthase | Clarkia breweri | | Pichersky (1996) |
| Myrcene/(E)-β-Ocimene | Arabidopsis | | Bohlmann (2000) |
| synthase | thaliana | | |
| Sesuniterpene synthases | | | |
| 5-epi Aristolochene synthase | Capsicum annuum | | Shin (1998) |
| Trichodiene synthase | Fusarium | Cyclization via NDP | Hobn (1989) |
| | sporotrichoides | intermediate | |
| Germacene C synthase | Lycopersicon | | Croteau (1998) |
| | esculentum | | |
| (E)-β-Famesene synthase | Menthapiperita | linear Croteau (1997) | |
| (E)-α-Bisabolene synthase | Abies grandis | High product specificity | Croteau (1998) |
| | | Cyclization via NDP | |
| | | intermediate | |
| Epi-Cedrol synthase | Artemisia annua | Cyclization via NDP | CroteaulBrodelius (1999) |
| | | intennediate | |
| δ-Selinene synthase | Abies grandis | Produces 52 diff. | Croteau (1998) |
| | | Sesquiterpenes | |
| γ-Humulene synthase | Abies grandis | Produces 34 diff. | Croteau (1998) |
| | | Sesquiterpenes | |
| 5-epi Aristolochene synthase | Nicotiana tabacum | | Chappell (1994) |
| Cadinene synthase (2) | Gossypium | High product specificity | Davisson/Heinstein |
| | arboreum | Cyclization via NDP(1995/1996) | |
| | | intermediate | |
| Pentalene synthase | Streptomyces sp. | | Cane (1994) |
| Trichodiene synthase | Myrothecium | Cyclization via NDP | Hohn (1998) |
| | roridum | intermediate | |
| Vetispiradiene synthase | Hyoscyamus | | Chappell (1995) |
| | muticus | | |
| Aristolochene synthase | P. roqueforti | | Hobn (1993) |
| Diterpene synthases | | | |
| Casbene synthase | Ricinus communis | | West (1994) |
| Taxadiene synthase | Taxus brevfolia | | Croteau (1996) |
| Abietadiene synthase | Abies grandis | Bifunctional | Croteau (1996) |
| ent-Kaurene synthase | Phaeosporia sp | Bifunctional: GGDP to ent- | Kamiya (2000) |
| | | kaurene | |
| ent-Kaurene synthase A | Maize | GGDP to CDP Briggs (1995) | |
| ent-Kaurene synthase A | Stevia rebaudiana | GGDP to CDP | Brandle |
| ent-Kaurene synthase A | Arabidopsis | GGDP to CDP Kamiya (1994) | |
| | thaliana | | |
| ent-Kaurene synthase A | Gibberella | GGDP to CDP Kamiya (1998) | |
| | fujikuroi | | |
| ent-Kaurene synthase B | Cucurbita maxima | CDP to ent-kaurene | Kamiya (1996) |
| ent-Kaurene synthase B | Stevia rebaudiana | CDP to ent-kaurene | Brandle (1999) |
| Triterpene synthases | | | |
| | (plant +microbial) | | |
| Squalene-hopene cyclase | Bradyrhizobium | Squalene cyclase | Kannenberg/Poralla |
| | japonicum | (1997) | |
| Squalene-hopene cyclase | Zymomonas | Squalene cyclase | Sprenger/Poralla |
| | mobilis | | (1995) |
| Lanosterol synthase | Candida albicans | Oxidosqualene cyclase | Kirsch (1990) |
| Lupeol synthase | Arabidopsis | Oxidosqualene cyclase | Matsuda (1998) |
| | thaliana | | |
| β-Amyrin synthase | Panax ginseng | Oxidosqualene cyclase | Ebizuka (1998) |
| Cycloartenol synthase | Pisum sativum | Oxidosqualene cyclase | Ebizuka (1997) |
| Lanosterol synthase | S. cerevisiae | Oxidosqualene cyclase | Bartel (1994) |
| Lupeol synthase | Olea erupea | Oxidosqualene cyclase | Ebizuka (1999) |
| Lupeol synthase | Taraxacum | Oxidosqualene cyclase | Ebizuka (1999) |
| | officinale | | |
| Cycloartenol synthase | Arabidopsis | Oxidosqualene cyclase | Bartel (1993) |
| | thaliana | | |
| Lanosterol synthase | Candida albicans | Oxidosqualene cyclase | Kirsch (1990) |
| Lupeol synthase | Arabidopsis | Oxidosqualene cyclase | Matsuda (1998) |
| | thaliana | | |
| β-Amyrin synthase | Panax ginseng | Oxidosqualene cyclase | Ebizuka (1998) |
| Cycloartenol synthase | Pisum sativum | Oxidosqualene cyclase | Ebizuka (1997) |
| Lanosterol synthase | S. cerevisiae | Oxidosqualene cyclase | Bartel (1994) |
| Lupeol synthase | Olea erupea | Oxidosqualene cyclase | Ebizuka (1999) |
| Lupeol synthase | Taraxacum | Oxidosqualene cyclase | Ebizuka (1999) |
| | officinale | | |
| Cycloartenol synthase | Arabidopsis | Oxidosqualene cyclase | Bartel (1993) |
| | thaliana | | |

Diterpene Synthases

Diterpene synthases catalyze the cyclization of GGDP to either macrocyclic or cyclohexanoic diterpenoids by two fundamentally different modes of cyclization. Non-aromatic macrocyclic diterpenes like e.g. casbene, cembrene or taxadiene are formed by a cyclization reaction similar to that of mono- and sesquiterpene cyclases. Synthesis of aromatic, cyclohexanoic diterpenes like e.g. abatiedadiene or ent-kaurene, involves the generation of copalyl diphosphate as an intermediate. This reaction cascade is initiated by protonation of the terminal double bond of GGDP followed by two internal additions and proton elimination, in a sequence similar to that catalyzed by triterpene cyclases. CDP is transformed into a variety of tricyclic and tetracyclic diterpenoids by ionization of the diphosphate ester and subsequent internal additions, rearrangements and quenching reactions (reviewed in McCaskill and Croteau, Adv. Biochem. Eng. Biotechnol. 1997;55:107-146; and Bohlmann et al., Proc. Natl. Acad. Sci. USA 1998;95:4126-4133).

More than 3,000 diterpenes have been characterized until present, but enzymes have only been cloned for the biosynthesis of four diterpenes. To date, genes have been cloned encoding casbene and taxadiene synthase for macrocyclic diterpene biosynthesis and ent-kaurene and abietadiene synthase for aromatic diterpenes biosynthesis (Mau and West, Proc. Natl. Acad. Sci. USA 1994;91:8497-8501; Stof er-Vogel et al., J. Biol. Chem. 1996;271:23262-23268; Kawaide et al., J. Biol. Chem. 2000;275:2276-2280; Wildung and Croteau, J. Biol. Chem. 1996;271:9201-9204).

Biosynthesis of aromatic diterpenes requires two enzyme functions that are either located in two different enzymes or in one bifunctional enzyme. In the case of the two-enzyme assisted ent-kaurene biosynthesis, ent-kaurene synthase A catalyzes the formation of CDP while ent-kaurene synthase B transforms CDP to ent-kaurene. Recently, a fungal bifunctional ent-kaurene synthase has been cloned from Phaeosporia sp. (See Table 1). The only abietadiene synthase gene known so far encodes a bifunctional enzyme cloned from Abies grandis.

Triterpene Synthases

Triterpene synthases cyclize the C30 carbon isoprene substrates squalene and 2,3-oxidosqualene to a variety of polycyclic products. Unlike class I cyclases, triterpene cyclases generate the reactive carbocation either by protonation of the terminal double bond of squalene or protonation and ring opening of the 2,3-epoxide group of 2,3-oxidosqualene. Squalene and 2,3-oxidosqualene are synthesized by ahead-to-head condensation of two FDP isoprene units and thus lack a reactive diphosphate ester. The cyclization reactions catalyzed by triterpene cyclases are one of the most complex one-step reactions known in either biochemistry or synthetic chemistry. Lanosterol synthase for example alters 20 bonds, forms 4 rings and sets 7 stereo centers to synthesize highly specific lanosterol from oxidosqualene (Corey et al., 1994, supra). Squalene hopene cyclase (Reipen, et al., Microbiology 1995;141:155-161) and β-amyrin synthase (Kushiro et al., Eur. J. Biochem. 1998;256:23 8-244) catalyze cyclization cascades that form pentacyclic triterpenoids.

Triterpenoids can be divided into steroidal and non-steroidal triterpenoids. They are produced in a variety of organisms and exhibit a variety of functions. In animals (in vertebrates, cholesterol derived from lanosterol), plants (cycloartenol (Corey et al., Proc. Natl. Acad. Sci. USA 1993;90:11628-11632)), yeast and fungi (lanosterol) are sterols important membrane constituents and, at the same time, serve as precursors for various hormones. The pentacyclic hopanoid and tetrahymenol lipids found many bacteria and function as reinforcers of cellular membranes. Although thousands of non-steroidal triterpenoids have been identified, mainly in plants, their function is yet unknown. Only recently, two enzymes encoding β-amyrin and lupeol synthase (Herrera et al., Phytochemistry 1998;49:1905-191 1) have been cloned. The best-characterized enzymes are squalene-hopene cyclase (structure known) and lanosterol synthase.

Terpenoid Structure Modifying Enzymes

Compared to the number of cloned terpene cyclases, little is known about the enzymes that further modify terpenoid skeletons. These additional modifications of ten lead to the physiological active terpenoid. Taxol biosynthesis, for example, involves first cytochrome P450-dependent hydroxylation of taxadiene, followed by acetylation and oxetane ring formation and then several subsequent oxygenation steps. Very recently, two enzymes catalyzing the third and the last step of taxol biosynthesis were cloned (Schoendorf and Croteau, Arch. Biochem. Biophys. 2000;374:371-380; Walker and Croteau, Proc. Natl. Acad. Sci. 2000;97:583-587).

Other modifying enzymes that have been cloned include an acetyltransferase and two P450 monooxygenases involved in trichoethecene biosynth, esis, several oxidases and a hydroxylase involved in gibberellin biosynthesis and a limonene hydroxylase (Lupien et al., Arch. Biochem. Biophys. 1999;368:181-192; Alexander et al., Appl. Environm. Microbiol. 1998;64:221-225; Hohn et al., Mol. Gen. Genet. 1995;248:95-102; Thomas et al., Proc. Natl. Acad. Sci. USA 1999;96:4698-4703; McCormick et al., Appl. Environm. Microbiol. 1996;62:353-359). Enzymes catalyzing the epoxidation of squalene to form 2,3-oxidosqualene have also been cloned (Favre and Ryder, Gene 1997; 189:119-126). Most of the modifying enzymes that have been cloned are P450 monooxygenases.

Table 2 shows a list of enzymes, involved in the biosynthesis of terpenoids, which are contemplated for modification according to the invention. Also provided are accession numbers to corresponding genes, any of which can be used in directed evolution of a terpenoid biosynthetic pathway according to the invention.

TABLE 2
Selection of Sequences Encoding Terpenoid Biosynthesis Enzymes

| ENZYME/PATHWAY | ORGANISM | ACCESSION NO. |
|---|---|---|
| S-linalool synthase | Clarkia brewer | U58314 |
| E-alpha-bisabolene synthase | Abies grandis | AF006195 |
| casbene synthase | Ricinus communis | L32 134 |
| pentalenene synthase | Streptomyces sp. | U05213 |
| 2,3-oxidosqualene-triterpenoid cyclase | Arbidopsis thaliana | U87266 |
| beta-Amyrin synthase | Panax ginseng | AB009030 |
| cycloartenol synthase | panax ginseng | AB009029 |
| kaurene synthase | Stevia rebaudiana | AF0973 10 |
| taxadiene synthase | Taxus brevifolia | U48796 |
| abietadiene synthase | Abies grandis | U50768 |
| kaurene synthase | Stvia rebaudiana | AF0973 11 |
| copalyl pyrophosphate synthase | Stevia rebaudiana | AF034545 |
| ent-kaurene synthase | Phaeosphaeria sp. | ABOO3 395 |
| sesquiterpene synthase | Artemisia annua | AF138959 |
| sesquiterpene cyclase | Capsicum annuum | AF212433 |
| delta-cadinen synthase | Gossypium hirsutum | U883 18 |
| d-selinene synthase | Abies grandis | U92266 |
| trichodiene synthase | Fusarium poae | U15658 |
| aristolochene synthase | Penicillium roqueforti | L05193 |
| sesquiterpene cyclase | Capsicum annuum | AF061285 |
| squalene synthase | Yarrowia lipolytica | AF092497 |
| squalene synthase | Botryococcus braunii | AF205791 |
| squalene synthase | Capsicum annuum | AF124842 |
| squalene epoxidase | Candida albicans | U69674 |
| squalene synthase | Candida utilis | AB012604 |
| squalene-hopene cyclase | Alicyclobacillus | AB007002 |
| | acidocaldarius | |
| hpnA, p1mB, hpnC, hpnD, hpnE | Zymomonas mobilis | AJ001401 |
| squalene epoxidase | Candida glabrata | AF006033 |
| squalene synthase | Candida albicans | D89610 |
| squalene epoxidase | Candida albicans | D88252 |
| squalene-hopene-cyclase | Z.mobilis | X73561 |
| squalene synthetase | S.cerevisiae | X59959 |

Selection and Cloning of Biosynthetic Genes

In a preferred embodiment, bacterial sesquiterpene and triterpene synthases are subjected to directed evolution. Also preferred are plant terpenoid synthase genes, although the large number of highly homologous terpenoid synthase isoforms in plants may make cloning of different genes and determination of product and substrate specificity somewhat laborious. The high product specificity of bacterial terpene cyclases compared to plant terpene cyclases render bacterial terpene cyclases especially suitable.

Sesquiterpene synthases appears to be the most flexible class of terpene synthases, possibly producing more than 7,000 different terpenoids with more than 200 different cyclic structures. It is therefore conceivable that the available sesquiterpene synthases can be easily evolved to accept a variety of polyprenyl substrate for the recombinant production of novel terpenoid structures. Triterpenoid cyclases catalyze one of the most complex biochemical reactions known in nature. At present, however, only a few triterpenoid cyclases catalyzing the synthesis of a handful of triterpenoids have been cloned. Plants and microorganisms produce thousands of different triterpenoids with yet unknown biological functions. Triterpenoids represent therefore a highly interesting terpenoid class for the discovery of novel pharmaceuticals and agrochemicals. Since triterpene cyclases appear to be internal membrane enzymes that release their hydrophobic products into the membrane, it is likely that these cyclases can be evolved to cyclize hydrophobic long-chain polyprenyls like phytoene (C40) as well.

Preferred genes for modification according to the invention, include but is not limited to those listed in Table 3, encoding for sesquiterpene and triterpene cyclases, as well as polyprene-chain synthases and modifying enzymes.

TABLE 3
Preferred Sequences Encoding Terpenoid Biosynthesis Enzymes

| Gene | Source | Comments | Accession No. |
|---|---|---|---|
| Polyprene-chain synthases | | | |
| FDP synthase (C15) | E. coil (ispA) | | D00694 |
| GGDP synthase (C20) | Erwinia uredevora (ertE) | Gene cloned | D90087 |
| Squalene synthase (C30) | Zymomonas mobilis | | AJ001401 |
| Dehydrosqualene synthase | Staphylococcus aureus (crtN) | | X73889 |
| (C30) | | | |
| Phytoene synthase (C40) | Erwinia uredevora (crtB) | Gene cloned | D90087 |
| Sesquiterpene synthases | | | |
| Pentalenene synthase | Streptomyces sp. | | U05213 |
| Triterpene synthases | | | |
| Squalene-hopene cyclase | Alicyclobacillus acidocaldarius | Homology group I | M73 834 |
| | Streptomyces coelicor | Homology group I | AL049485 |
| | Zymomonas mobills | Homology group II | X73561 |
| | Bradyrhizobiumjaponicum | Homology group II | X86552 |
| | Rhodopseudomonas palustris | Homology group II | Y09979 |
| Lanosterol synthase | Alicyclobacillus acidoterrestris | Homology group I | X89854 |
| Other enzymes | | | |
| Spheroidene monooxygenase | Rhodobacter capsulatus (crtA) | Genes cloned | X52291 |
| | Rhodobacter sphaeroides (crtA) | AJ010302 | |
| Squalene epoxidase | S. cerevisiae (ERG1) | M64994 | |

Pentalenene synthase can be cloned from Streptomyces sp. genomic DNA based on the known nucleotide sequence. Additional homologous sesquiterpene cyclase genes for DNA-shuffling can be isolated by PCR from other Streptomyces strains that have been reported to produce pentalenolactone antibiotics (e.g. S. arenae, S. omiyaensis, S. albofaciens, S. viridifaciens).

Bacterial triterpenoid cyclases can be divided into two homology groups based on nucleotide sequence identity (>80% identity) (see Table 1). These triterpenoid cyclases can be cloned from genomic DNA of the respective strains.

Enzymes necessary for the biosynthesis in E. coli of FDP, GGDP, squalene, dehydrosqualene and phytoene that are required as substrates for the terpene cyclases, can also be cloned from genomic DNA of readily available bacterial strains. Phytoene synthase and GGDP synthase have already been cloned during work on directed evolution of carotenoid biosynthetic pathways. (See Examples).

Squalene epoxidase necessary for the production of oxidosqualene can be cloned from S. cerevisiae and carotenoid monooxygenases that introduce oxygen functions in polyprenyl substrates have been cloned from two Rhodobacter strains during prior work on carotenoid biosynthesis. (See Examples)

Suitable bacterial gene sources are available from strain collections, and functional gene expression in E. coli has been reported for selected genes. Genes can initially be cloned into pUCmod, which contains an optimized SD-sequence downstream of the lac-promoter and an optimized multiple cloning site. The complete unit consisting of promoter and biosynthetic gene can easily be excised from pUCmod and cloned into pACmod for pathway assembly.

Literature describing the structure and/or function of terpendoid genes can be found in, e.g., Dudareva N., et al., Plant Cell Jul. 8, 1996 (7): 1137-48; Bohlmann, J. et al., Proc Natl Acad Sci U S A Jun. 9, 1998;95(12):6756-61; Proc Natl Acad Sci U S A Aug. 30, 1994;91(18): 8497-501; Cane D E et al., Biochemistry May 17, 1994;33(19):5846-57; Colby S M et al., J Biol Chem Nov. 5, 1993; ;268(31):23016-24; Back K, et al., J Biol Chem Mar.31, 1995;270(13):7375-81; Kawaide H, et al., J Biol Chem Aug. 29, 1997;272(35):21706-12; Bohlmann J, et al., Arch Biochem Biophys Mar. 15, 2000; 375(2):261-9; Yuba A, et al., Arch Biochem Biophys Aug. 15, 1996;332(2):280-7; Inoue T, et al., Biochim Biophys Acta Jan. 2, 1995;1260(1):49-54; Back K et al., Arch Biochem Biophys Dec. 1994; 315(2):527-32; Vogel B S et al., J Biol Chem Sep. 20, 1996;271(38):23262-8; Wildung M R, et al., J Biol Chem Apr. 19, 1996;271(16):9201-4; Steele C L, et al., J Biol Chem Jan. 23, 1998; 273(4):2078-89; Bohlmann J, et al., Arch Biochem Biophys Aug. 15, 1999;368(2):232-43; Abe I, et al., Proc Natl Acad Sci U S A Sep. 26, 1995;92(20):9274-8; Hanley K M, et al., Plant Mol Biol Mar. 30, 1996;(6):1139-51; Back K, et al., Plant Cell Physiol Sep. 1998;39(9):899-904; Cane D E, et al., Arch Biochem Biophys Aug. 1, 1993;304(2):415-9; Cane D E, et al., Arch Biochem Biophys Jan. 1993; 300(1):416-22; Huang K X, et al., Protein Expr Purif Jun. 13, 1998;(1):90-6; Merkulov S, et al., Yeast Feb. 16, 2000;(3):197-206; Jennings S M, et al., Proc Natl Acad Sci U S A Jul. 15, 1991; 88(14):6038-42; Corey E J, et al., Proc Natl Acad Sci U S A Mar. 15, 1994;91(6):2211-5; Favre B, et al., Gene Apr. 11, 1997;189(1):119-26; Sakakibara J, et al., J Biol Chem Jan. 6, 1995;270(1):17-20; Tippelt A, et al., Biochim Biophys Acta Mar. 30, 1998;1391(2):223-32; Reipen I G, et al., Microbiology Jan. 1995;141 (Pt 1):155-61; Full C, et al., FEMS Microbiol Lett Feb. 15, 2000; 183(2):221-4; Perzl M, et al., Microbiology Apr. 1997;143 (Pt 4):1235-42; Ochs D, et al., J Bacteriol Jan. 1992;174(1):298-302; Nakashima T, et al., Proc Natl Acad Sci U S A Mar. 14, 1995; 92(6):2328-32; Kushiro T, et al., Eur J Biochem Aug. 15, 1998;256(1):238-44; Shibuya M, et al., Eur J Biochem Nov. 1999;266(1):302-7; Morita M, et al., Biol Pharm Bull Jul. 20, 1997;(7):770-5; Sung C K, et al., Biol Pharm Bull Oct. 18, 1995;(10):1459-61; Herrera J B, et al., Phytochemistry Dec. 1998; 49(7):1905-11; Kusano M, et al., Biol Pharm Bull Jan. 18, 1995;(1):195-7; Corey E J, et al., Proc Natl Acad Sci U S A Dec. 15, 1993;90(24):11628-32; Okada S, et al., Arch Biochem Biophys Jan. 15, 2000; 373(2):307-17; Kelly R, et al., Gene Mar. 15, 1990;87(2):177-83; Corey E J, et al., Biochem Biophys Res Commun Feb. 15, 1996;219(2):327-31; Tao Y, et al., J Biol Chem Oct. 13, 1995; 270(41):23984-7; Sloane D L, et al., Gene Aug. 19, 1995;161(2):243-8; Richman A S, et al., Plant J Aug. 19, 1999;(4):411-21; Tudzynski B, et al., Curr Genet Sep. 1998;34(3):234-40; Kawaide H, et al., J Biol Chem Jan. 28, 2000;275(4):2276-80; Yamaguchi S, et al., Plant Physiol Apr. 1998; 116(4):1271-8; Tudzynski B, et al., Fungal Genet Biol Dec. 25, 1998;(3):157-70; Coles J P et al., Plant J Mar. 17, 1999;(5):547-56; Thomas S G, et al., Proc Natl Acad Sci U S A Apr. 13, 1999; 96(8):4698-703; Shimizu N, et al., J Bacteriol Mar. 1998;180(6):1578-81; Zhang Y W, et al., Biochemistry Sep. 22, 1998;37(38):13411-20; Apfel C M, et al., J Bacteriol Jan. 1999;181(2): 483-92; Schmidt CO, et al., Arch Biochem Biophys Apr. 15, 1999;364(2):167-77; Bouwmeester H J, et al., Phytochemistry Nov. 1999;52(5):843-54; Mercke P, et al., Arch Biochem Biophys Sep. 15, 1999; 369(2):213-22; Colby S M, et al., Proc Natl Acad Sci U S A Mar. 3, 1998;95(5):2216-21; Bohlmann J, et al., J Biol Chem Aug. 29, 1997;272(35):21784-92; Walker K, et al., Arch Biochem Biophys Feb. 15, 2000;374(2):371-80; Walker K, et al., Proc Natl Acad Sci U S A Jan. 18, 2000; 97(2):583-7; Chen X Y, et al., Arch Biochem Biophys Dec. 20, 1995;324(2):255-66, Jia J W, et al., Arch Biochem Biophys Dec. 1, 1999;372(1):143-9; Wise M L, et al., J Biol Chem Jun. 12, 1998; 273(24):14891-9; Shimizu N, et al., J Biol Chem Jul. 31, 1998;273(31):19476-81; Crock et al., Proc Natl Acad Sci U S A Nov. 25, 1997;94(24):12833-8; Okada K, et al., Eur J Biochem Jul. 1, 1998; 255(1):52-9; Chen X Y, et al., J Nat Prod Oct. 1996;59(10):944-51; Hohn T M, et al., Arch Biochem Biophys Nov. 15, 1989;275(1):92-7; Proctor R H, et al., J Biol Chem Feb. 25, 1993;268(6):4543-8; Hua L, et al., Arch Biochem Biophys Sep. 15, 1999;369(2):208-12; Tachibana A, et al., Eur J Biochem Jan. 2000;267(2):321-8; Fekete C, et al., Mycopathologia 1997;138(2):91-7; Trapp S C, et al., Mol Gen Genet Feb. 1998;257(4):421-32; Alexander N J, et al., Appl Environ Microbiol Jan. 1998; 64(1):221-5; Hohn T M, et al., Gene Jun. 30, 1989;79(1):131-8; Hohn T M, et al., Mol Gen Genet Jul. 22, 1995;248(1):95-102; McCormick S P, et al., Appl Environ Microbiol Feb. 1996;62(2):353-9.

### Directed Evolution of Terpenoid Biosynthetic Pathways

The above listed enzymes, as well as other enzymes of potential interest, may be applied in the context of the invention to produce known or novel terpenoids in a more efficient manner. For example, the modular expression vectors pUCmod and pACmod (See section entitled “Carotenoids” below), can be used to clone and assemble terpenoid biosynthetic pathways in E. coli. Similarily, biosynthetic genes that useful for the production of polyprenyl diphosphate precursors in E. coli can be cloned into pACmod in such a way that each gene is under the control of either an optimized lac- or tac-promoter. Co-transforming E. coli cells, synthesizing polyprenyl diphosphate precursors, with shuffled terpenoid cyclases genes cloned into pUCmod can create pathway libraries.

To establish terpenoid biosynthesis in E. coli, bacterial terpenoid cyclases can be transformed into E. coli cells producing FDP, GGDP or squalene depending on the desired type of cyclase. Terpenoid production in shaking flasks and microtiter plates can be investigated as described below. Optimal cultivation conditions can be established both for larger scale terpenoid production and for terpenoid biosynthesis in microtiter plates.

Specific Embodiments of Directed Evolution of Terpene Synthases

Shuffling of either the homologous Streptomyces sesquiterpene synthases or the two groups of homologous bacterial triterpene synthase followed by transformation of the shuffled genes into E. coli cells producing polyprenyl substrates (or by combination of cell lysates) can create libraries of terpenoid pathways.

Streptomyces Sesquiterpene Synthase. This embodiment describes shuffling of either the homologous Streptomyces sesquiterpene synthases or the two groups of homologous bacterial triterpene synthase followed by transformation of the shuffled genes into E. coli cells producing polyprenyl substrates (or by combination of cell lysates) to create libraries of terpenoid pathways. Sesquiterpene and triterpene synthase variants, producing in E. coli cyclization products other than those produced by the wild type enzyme from their natural substrates, can be created by DNA shuffling. This approach allows exploring the diversity of possible cyclization products that can be produced from FDP by a sesquiterpene cyclase and from squalene by a triterpene synthase. It is likely that sesquiterpene and triterpene synthase variants can be obtained which synthesize novel types of terpenes in E. coli that are either not found in nature or are not available from natural sources.

Terpene synthases for novel substrates. This embodiment describes shuffling of terpene synthase genes to create variants which cyclize polyprenyl substrates other than their natural substrates for the biosynthesis of new types of terpenes. Libraries of shuffled sesquiterpene synthases can be transformed into E. coli cells producing GGDP for the production of novel cyclic terpene structures. Cyclization of GGDP by sesquiterpene cyclases has not been found so far, however, the homology of sesquiterpene cyclases with other cyclases makes it likely that variants may be capable to cyclize GGDP and even produce new diterpene structures. Triterpene synthases, which cyclize polyprenyl substrates containing no reactive diphosphate ester group, can be adapted to cyclize unnatural substrates such as phytoene and dehydrosqualene. Thereby, completely novel types of terpenes not found in nature can be created. In addition, triterpene cyclase variants may be obtained that will cyclize the polyprenyl diphosphate GGDP as substrate and thus, possibly synthesize novel types of diterpenes.

Terpene synthases for modified substrates. This embodiment describes shuffling of terpene synthase genes to generate variants that cyclize modified polyprenyl substrates for the biosynthesis of new types of terpenes. Many of the biologically active terpenoids contain additional modifications to their cyclic terpene skeleton. The introduction of oxygen functions is the most common modification, which is of ten catalyzed by P450 monooxygenases. However, only few such modifying enzymes, mainly oxygenases, have been cloned so far. To introduce oxygen functions into terpene structures, one possible strategy is to chose carotenoid monooxygenases (spheroidene monooxygenase crtA from Rhodobacter). Preliminary studies has shown that these monooxygenases are evolvable to oxygenize different substrates derived from phytoene. (See below). In vitro evolution of crtA to synthesize novel acyclic C40 and C30 oxo-carotenoids are described in other embodiments herein. Pathways for the synthesis of oxygenated phytoene, squalene or dehydrosqualene created in this project can thus, similarly, be used for the creation of novel oxygenated terpenoid structures. Libraries of shuffled wild type triterpene synthases or triterpene synthase variants can be transformed in E. coli cells producing oxygenated polyprenyl substrates and screened for new cyclic terpenoids.

Finally, the discovered novel pathways can be collected and the clones and genes preserved. For analyses such as preliminary structural classification, larger amounts of novel terpenoids can be synthesized in E. coli for further analysis. In some cases, optimization of a promising cyclase variant through additional rounds of in vitro evolution may be advantageous.

### Development of Terpenoid Analytical and Screening Methods.

As terpenoids do not have any light absorbing or fluorescence properties, analysis of terpene biosynthesis relies either on the use of radio-labeled substrates and radio-GC/HPLC or on GC/HPLC-MS. Both radio-GC and GC-MS are the predominant methods described for terpene analysis in literature. However, HPLC-MS has also been used, especially for the less or non-volatile terpenoids with 15 or more carbon atoms (Bohlmann et al., PNAS 1998, supra; Corey et al., Proc. Natl. Acad. Sci. USA 1994;91:2211-2215; Thomas et al., Proc. Natl. Acad. Sci. USA 1999;96:4698-4703). Hence, HPLC-MS methods for the analysis and quantification of terpenoids can be developed. GC-MS can be used for routine analysis of biosynthesis of known terpenoids. For both HPLC and GC analysis, methods described in literature can be adapted to the actual analytical needs and to existing equipment. Methods for terpenoid extraction and sample preparation for GC/LC-MS analysis is preferably developed based on published material. Special emphasis should be put on the development of methods requiring only few simple steps that are adaptable to high-throughput sample analysis. Furthermore, known terpenes can be isolated as standards for GC/LC-MS analysis according to published methods. The wealth of published terpenoid mass spectra and of those deposited in the NIST database can also be recruited for terpenoid identification. In some cases, structural identification by high-resolution NMR and mass spectrometry may become necessary.

Central to terpenoid pathway breeding will be the development of high-throughput screening methods for rapid identification of new terpenoid biosynthetic capabilities in large E. coli libraries. As described above, LC-MS would be the method of choice for terpenoid analysis. Samples need to be directly injected into the LC-MS from either microtiter plates or deep well plates. Biosynthesis of new terpenoids can initially be identified by mass selective detection and retention times of peaks compared to terpenoid standards.

Because of the long analysis time required for each individual sample compared to the fast parallel readings by a plate reader, development of a pooling strategy may be crucial to reduce sample numbers and thus, analysis time. Such a pooling strategy could involve pooling, for example, 50 clones (or extracts thereof ) of a library for the initial analysis of 100-200 of such pooled samples. Upon identification of pools containing novel terpenoid structures, the initial 50 clones of a pool can then be subdivided into pools of 10 clones for further analysis. After one or two additional rounds of pooling and subdividing, E. coli clones that synthesize novel terpenoid structures should be identifyable.

Libraries can be transferred from agar plates to microtiter plates for cell growth and terpenoid or precursor production. In order to screen libraries of shuffled terpenoid cyclases with several different polyprenyl substrates for the synthesis of novel terpenoid structures, an in vitro method for terpenoid synthesis using E. colicell extracts can be developed. Extracts from E. coli cells producing the polyprenyl substrates for terpenoid cyclases will be combined with those obtained from the individual E. coli clones of the shuffled terpenoid cyclase library. The same cell lysate prepared from a batch of recombinant E. coli cells producing a polyprenyl substrate can be used to screen the entire library of shuffled terpenoid cyclases, thereby allowing homogenous terpenoid biosynthesis throughout the library. However, if this in vitro approach does not lead to the synthesis of sufficient amounts of terpenoids for LC-MS analysis, which may be possible for the biosynthesis of the membrane dissolved hydrophobic tri- and tetraterpenes, in vivo terpenoid libraries can be created.

Terpenoids can be extracted from E. coli cells or lysates with solvents such as pentane, hexane and acetone. For instance, protocols developed for 96-well plate carotenoid extraction as described below can be adapted for terpenoid extraction. As an alternative, solid phase extraction can be investigated for sample preparation for LC-MS analysis.

### Carotenoids

### General

Carotenoids represent the major class of natural pigments. More than 600 different carotenoids have been identified in bacteria, fungi, algae, plants and animals (Staub, O., In: Pfander, H. (ed.), Key to Carotenoids, 2^{nd} ed., Birkhäuser Verlag, Basel). They function as accessory pigments in photosynthesis, as antioxidants, as precursors for vitamins in humans and animals and as pigments for light protection and species specific coloration. Light absorption properties of the predominantly yellow to red carotenoids are determined by their delocalization and isomerisation state (Packer, L., Meth. Enzymol., 1992, 214, Part B). Carotenoids are of interest, e.g., for pharmaceuticals, food colorants, and animal feed and nutrient supplements. The discovery that these natural products can play an important role in the prevention of cancer and chronic disease (mainly due to their antioxidant properties) and, more recently, that they exhibit significant tumor suppression activity due to specific interactions with cancer cells has boosted interest in their pharmaceutical potential (Bertram, J. S., Nutr. Rev., 1999;57:182-191; Singh, et al., Oncology, 1998;12:1643-168; Rock, C. L., Pharmacol. Ther., 1997;75:185-197; Edge, et al., J. Photochem. Photobiol., 1997;41:189-200).

At present, carotenoids are commercially produced as antioxidants, food colorants, vitamin A precursors and as animal food additives (e.g., in aqua farming and poultry industry, Krinski, N. I., Pure Appl. Chem., 1994, 66:1003-1010 and Polazza, P. and Krinski, N. I., Meth. Enzymol., 1992, 213:403-420). Although the use of carotenoids as natural food colorants, as antioxidants in cancer prevention, and immune modulators will increase, only a few carotenoids can be obtained in useful quantities by chemical synthesis, extraction from their natural sources or microbial fermentation (Johnson, et al., Adv. Biochem. Eng. Biotechnol., 1995;53:119-178). A number of carotenogenic genes have therefore been cloned from microorganisms and plants and expressed in E. coli, thereby allowing the recombinant biosynthesis of different acyclic, cyclic caroteioids and oxo-carotenoids (Misawa, N. and Shimada, H., J. Biotechnol., 1998;59:169-181 and Hirschberg, J., In: Carotenoids: Biosynthesis and Metabolism, Vol. 3, Carotenoids, G. Britton, Ed. Basel: Birkhäuser Verlag, 1998; 148-194).

Because of the availability of various biosynthetic genes from different organisms involved in the production of a wide array of carotenoids, functional expression of most genes in suitable host cells, and their biotechnological importance, carotenoid biosynthesis is a particularly useful application of the invention. Also, the light absorption properties of carotenoids allow for convenient high-throughput screening to identify new or desired carotenoids, or cells that produce increased concentrations of carotenoids.

### Carotenoid Biosynthetic Genes

Various carotenoids can be produced in recombinant microorganisms by combining biosynthetic genes from different organisms to create biosynthetic pathways. During the past 10 years, many microbial and plant enzymes of the first part of carotenoid, biosynthesis including complete bacterial biosynthesis pathways, have been cloned. In addition, enzymes responsible for further carotenoid modification have been characterized on a molecular level. The few exceptions are β-carotene ketolases from Agrobactrium, Haematococcus, Alcaligenes and Synechocystis, β-carotene hydroxlyases and glucosyl transferases from Erwinia and other microorganisms and plants, and neurosporene and lycopene modifying enzymes from phototropic bacteria. Two enzymes (β-cyclohexenyl expoxidase and capsanthin-capsorubin synthase) have been recently cloned which catalyze the synthesis of capsanthin and capsorubin through epoxidation of β-carotene at position C5-C6 to violaxanthin followed by a subsequent ring contraction. In addition, hydroxylases have also been cloned from plants and fungi, β-lycopene cyclases have been cloned from various microorganisms and plants, and ε-lycopene cyclases have been cloned from plants.

Any of these genes can be used in directed evolution of a carotenoid biosynthetic pathway of the invention. These genes, when isolated from bacteria, receive a “crt” designation. However, unless referring to a specific gene or gene product, that designation used herein refers to a gene function, whether or not the gene is bacterial or eukaryotic.

Most genes involved in carotenoid biosynthesis could be functionally expressed in E. coli or other microorganisms. This, and clustering of many bacterial biosynthesis genes in operons, allowed for the cloning of new biosynthetic genes and their functional characterization through complementation in recombinant E. coli or in mutant strains deficient in carotenoid production, e.g., Rhodobacter. Many carotenogenic genes employed in recombinant biosynthesis can be derived from either Rhodobacter or Erwinia species (Armstrong, G. A. and Hearst, J. E., FASEB J., 1996, 10:228-237 and Sandmann, G., Eur. J. Biochem., 1994, 223:7-24).

At present more than 150 genes for 24 carotenogenic enzymes (crt) have been isolated from bacteria, plants, algae and fungi that can be used to engineer a variety of diverse carotenoids in recombinant microorganisms (Table 4) (for an exhaustive list, see, Hirschberg et al., Pure and Appl. Chem., 1997, 69:2151; see also, Hirschberg In: Carotenoids: Biosynthesis and Metabolism, Vol. 3, in Carotenoids, 2^{nd} Ed., 1997, Basel: Birkhäuser Verlag, Table 2, pp. 184-191, which is specifically incorporated herein by reference).

TABLE 4
Selection of Genes Encoding Carotenoid Biosynthesis Enzymes

| Enzyme | Gene | Organism | Accession No. |
|---|---|---|---|
| ASSEMBLY OF CAROTENOID BACKBONE | | | |
| GGPP-synthase | crtE | Erwinia uredovora | D90087 |
| | crtE | Synechocystis PCC6803 | D90899 |
| | a1-3 | Neurospora crassa | X53979 |
| | Ggps | Arabidopsis thaliana | L25813 |
| Phytoene synthase | crtB | Agrobacterium aurantiacum | D58420 |
| | crtB | Synechocystis PCC6803 | X69172 |
| | a1-2 | Neurospora crassa | L27652 |
| | Psy | Arabidopsis thaliana | L25812 |
| Dehydrosqualene synthase (C30 | crtM | Staphylococcus aureus | X73889 |
| carotenoids) | | | |
| BIOSYNTHESIS OF ACYCLIC CAROTENOIDS | | | |
| Phytoene desaturase | | | |
| two desaturations | crtP | Synechocystis PCC6803 | X62574 |
| | Pds1 | Arabidopsis thaliana | L16237 |
| three desaturations | crt1 | Rhodbacter capsulatus | Z11165 |
| four desaturations | crt1 | Erwinia uredovora | D90087 |
| up to five desaturations | al-i | Neurospora crassa | M57465 |
| ζ-carotene desaturase | crtQ | SynechocystisPCC6803 | X62574 |
| | crtQ | Capsicum annuum | X68058 |
| Dehydrosqualene desaturase (C30 | crtN | Staphylococcus aureus | X73889 |
| carotenoids) | | | |
| Hydroxyneurosporene synthase | crtC | Rhodobacter sphaeroides | X82458 |
| | crtC | Rubrivivax gelatum | U73944 |
| Methoxyneurosporene desaturase | crtD | Rho dbacter capsulatus | Zi 1165 |
| | crtD | Rubrivivax gelatum | U73944 |
| Hydroxyneurosporene-O-methyltransferase | crtF | Rhodobacter sphaeroides | X82458 |
| Spheroidene monooxygenase | crtA | Rhodbacter capsulatus | Z11165 |
| BIOSYNTHESIS OF CYCLIC CAROTENOIDS | | | |
| Lycopene-β-cyclase | crtY | Erwinia uredovora | D90087 |
| | crtY | Synechocystis PCC6803 | X74599 |
| | CrtL-b | Arabidopsis thaliana | Z29211 |
| Lycopene-ε-cyclase | CrtL-e | Arabidopsis thaliana | U50738 |
| β-carotene hydroxylase | crtZ | Agrobacterium aurantiacum | D58420 |
| | CrtR-b1 | Capsicum annum | Y09225 |
| Zeaxanthin glucosylase | crtX | Erwinia herbicola Ehof | M87280 |
| β-carotene C(4) oxygenase | crtW | Agrobacterium aurantiacum | D58420 |
| | crtO | Synechocystis PCC6803 | D64004 |
| | crtW | Alcaligenes PC1 | D58422 |
| | CrtO/Bkt | Haematococcus pluvialis | X86782/D45881 |
| Zeaxanthin epoxidase | Zepi1 | Arabidopsis thaliana | T45502 |
| Violaxanthin deepoxidase | Vde1 | Arabidopsis thaliana | N37612 |
| Violaxanthin cleavage | Vp14 | Zea mays | U95953 |
| Capsanthin/capsorubin synthase | Ccs | Capsicum annum | X77289 |
| β-carotene desaturase | crtU | Streptomyces griseus | X95596 |

Complete carotenoid biosynthesis pathways have been cloned from a number of bacteria, where the biosynthesis enzyme genes are arranged in gene cluster (reviewed in Armstrong, Ann. Rev. Microbiol., 1997, 51:629; Sandmann, Eur. J. Biochem., 1994, 223:7). The pathways Erwinia and Rhodobacter for the synthesis of zeaxanthin diglucoside and the acyclic xanthophylls speroidene and spheroidenone, respectively, were the first from which all involved enzymes have been identified (Armstrong et al., Mol. & General Gene., 1989, 216:254; Lang et al., J. Bacteriol., 1995, 177:2064; Lee and Liu, Mol. Microbiol., 1991, 5:217; Misawa et al., J. Bacteriol., 1990, 172:6704).

Various techniques have been applied for cloning of carotenogenic genes (Hirschberg, J., In: Carotenoids: Biosynthesis and Metabolism, Vol. 3, Carotenoids, G. Britton, Ed. Basel: Birkhäuser Verlag, 148-194, 1998). Functional color complementation in E. coli expressing carotenogenic genes from Erwinia has been used successfully for the cloning of a variety of microbial and plant carotenogenic genes (Verdoes et al., Biotech. and Bioeng., 1999, 63:750; Zhu et al., Plant and Cell Physiology, 1997, 38:357; Kajiwara et al., Plant Mol. Biol., 1995, 29:343; Pecker et al., Plant Mol. Biol., 1996, 30:807). Recent advances in plant (including cyanobacteria) genomics and the use of cyanobacteria as models of plant carotenogenesis resulted in the identification of nearly all enzymes involved in plant carotenoid biosynthesis (reviewed in Hirschberg et al., Pure and Applied Chemistry, 1997, 69:215 1; Cunningham and Gantt, Ann. Rev. of Plant Physiol. and Plant Mol. Biol., 1998, 49:557). In contrast, cloned enzymes of bacterial carotenoid biosynthesis cover only the main routes.

Genes encoding the early carotenoid biosynthesis enzymes GGDP synthase, phytoene synthase and phytoene desaturase account for more than half of all cloned carotenogenic genes. Different phytoene desaturase genes are available that introduce two, three, four or five double bonds into phytoene to produce ξ-carotene (plant, cyanobacteria, algae) (Bartley et al., Eur. J. of Biochem., 1999, 259:396), neurosporene (Rhodobacter) (Raisig et al., J. Biochem., 1996, 119:559), lycopene (most eubacteria and fungi) (Verdoes, et al., Biotech. and Bioeng., 1999, 63:750; RuizHidalgo et al., Mol. & Gen. Genetics, 1997, 253:734) or 3,4-didehydrolycopene (Neurospora crassa) (Schmidhauser et al., Mol. and Cell Biol., 1990, 10:5064), respectively.

Lycopene-β-cyclases catalyzing β-ring formation have been cloned from a number of bacteria and plants and genes encoding lycopene-ε-cyclases have been isolated from plants (Cunningham et al., Plant Cell, 1996, 8:1613; Schnurr et al., Biochem. J., 1996, 315:869; Matsumura et al., Gene, 1997, 189:169; Cunningham et al, FEBS Lett., 1993, 328:130). While dicyclic products are formed by the β-lycopene cyclase, plant ε-lycopene cyclases usually synthesize monocyclic ε, ψ-carotene with the exception of lettuce ε-cyclase that forms ε,ε-carotene (Cunningham and Gantt, Ann. Rev. of Plant Physiol. and Plant Mol. Biol., 1998, 49:557). To date only β-ring modifying enzymes have been cloned, including a number of β-carotene C(3) hydroxylases from bacteria and plants (Linden, Biochimica et Biophysica Acta-Gene Structure and Expression, 1999, 1446:203; Bouvier, Biochimica et Biophysica Acta-Lipids and Lipid Metabolism, 1998, 1391:320; Pasamontes et al., Gene, 1997, 185:35) and β-carotene C(4) ketolase or oxygenases from bacteria and algae (Kajiwara et al., Plant Mol. Biol., 1995, 29:343; Misawa et al., Biochem. and Biophy. Research Comm., 1995, 209:867; FernandezGonzalez et al., J. Biol. Chem., 1997, 272:9728; Lotan and Hirschberg, FEBS Lttr., 1995, 364:125; Misawa et al., J. of Bacteriol., 1995, 177:6575). Plant genes were identified encoding zeaxanthin C(5, 6)epoxidase and violaxanthin C(5, 6) deepoxidase involved in the violaxanthin cycle and pepper capsanthin/capsorubin synthase catalyzing κring formation from the 3 -hydroxy-5,6-epoxy-β-rings in violaxanthin and antheraxanthin (see, Cunningham and Gantt, Ann. Rev. of Plant Physiol. and Plant Mol. Biol., 1998, 49:557).

Enzymes involved in acyclic carotenoid biosynthesis have so far only been cloned from phototrophic bacteria for the xanthophyll synthesis (Armstrong et al., Mol. & Gen. Gene., 1989, 216:254; Lang et al., J. of Bacteriol., 1995, 177:2064; Ouchane et al., J. Biol. Chem., 1997, 272:1670; Komori et al.Biochem., 1998, 37:8987). Recent additions to the collection of carotenogenic genes are dehydrosqualene synthase and desaturase from Staphylococcus aureus for the synthesis of the C30-carotenoid 4,4′-diaponeurosporene (Wieland et al., J. Bacteriol., 1994, 176:7719) and a β-carotene desaturase from Streptomyces griseus for the synthesis of isorenieratene containing aromatic end groups (Schumann et al., Mol. & Gen. Gene., 1996, 252:658; Krugel et al., Biochimica et Biophysica Acta-Mol. and Cell Biol. of Lipids, 1999, 1439:57).

U.S. Pat. No. 5,744,341 discloses eukaryotic genes encoding C-cyclase, isopentenyl pyrophosphate isomerase, and β-carotene hydroxylase, as well as vectors containing these genes.

The following carotenoid biosynthesis genes have been cloned.

crtE: GGPP-synthase from R. capsulatus and E. uredovora

crtB: phytoene synthase from R. capsulatus and E. uredovora

crtI: phytoene desaturase from E. uredovora and E. herbicola

crtY: lycopene cyclase from E. uredevora and E. herbicola

crtA: spheroidene monooxygenase from R. capsulatus and R. spaeroides

crtO: β-C4-ketolase (oxygenase) from Synechocistis sp.

crtW: β-C4-ketolase from Algaligenes sp., A. aurantiacum

crtD: methoxyneurosporene desaturase from R. capsulatus and R. spaeroides

crtX: zeaxanthin glucosyl transferase from E. uredovora and E. herbicola

crtZ: β-carotene hydroxylase from E. uredovora and E. herbicola

crtU: β-carotene desaturase from S. griseus

crtM: dehydrosqualene synthase from S. aureus

crtN: dehydrosqualene desaturase from S. aureus.

### Directed Evolution of Carotenoid Biosynthetic Pathways

The invention provides novel biosynthetic capacities by directed evolution of selected biosynthesis genes from different sources and subsequent complementation host cell strains that express carotenoid precursor biosynthetic genes, and optimally other carotenoid biosynthetic genes.

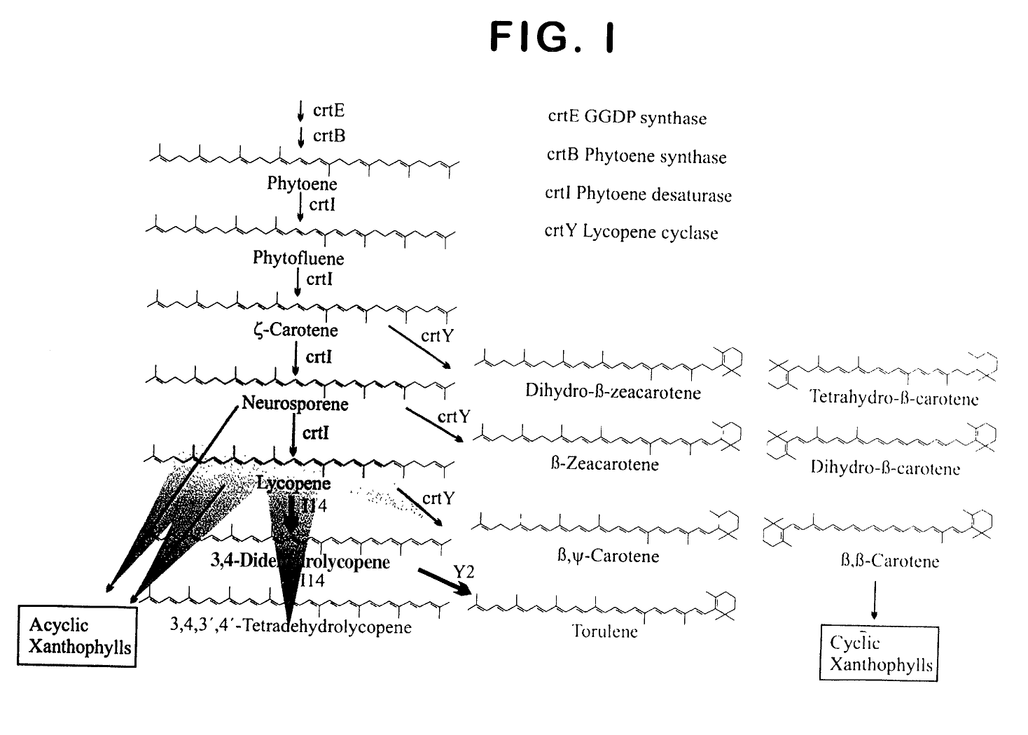

Carotenoids are derived from the universal isoprenoid biosynthesis pathway. Phytoene represents the first carotenoid of the pathway and is synthesized by a head-to-tail condensation of two C20 building blocks geranylgeranyl-diphosphate (GGPP). Enzymes necessary for the synthesis of GPP and phytoene, GGGP-synthase (crtE) and phytoene synthase (crtB) have been cloned from microorganisms as well as from plants. Starting from phytoene, three subsequent desaturation reaction result into the formation of neurosporene (Rhodobacter) or four desaturation reactions lead to the synthesis of lycopene (in cyclic carotenoid producing organisms). While desaturation is catalyzed by a single enzyme, phytoene desaturase (crtI), in bacteria and fungi, two enzymes (crtP and crtQ) desaturate phytoene via ξ-carotene to lycopene in cyanobacteria, algae, and plants. Cyclization of lycopene to β-carotene or α-carotene as in plants is catalyzed by homologous lycopene-β or lycopene-ε-cyclases. Species specific modifications of neurosporene, lycopene, diapocarotenoids, and carotene leads to the enormous diversity of carotenoids and oxygen-containing xanthophylls found in nature. A summary of current knowledge in carotenoid biosynthesis is found in Armstrong, G. A., Annu. Rev. Microbiol., 1997, 51:629-59; Cunningham, F. X. and Gantt, E., Annu. Rev. Plant Physiol. Plant Mol. Biol., 1998, 49:557-83; Armstrong, G. A. and Hearst, J. E., FASEB J., 1996, 10:228-237; Sandmann, G., Eur. J. Biochem., 1994, 223:7-24; and Armstrong, G. A., J. Bacteriol, 1994, 176:4795-4802. FIG. 1 outlines important carotenoid biosynthesis pathways known at present.

As noted above, a “phytoene desaturase” is an enzyme that introduces two desaturations in phytoene to produce ζ-carotene, as in plants and cyanobacteria; three desaturations to produce neurosporene, as in Rhodobacter; or four desaturations to produce lycopene, as in Erwinia and other photosynthetic bacteria (Garcia-Asua et al., Trends Plant Sci., 1998, 3:445-449). The desaturase from Neurospora crassa introduces five double bonds into phytoene to synthesize 3,4 didehydrolycopene (Bartley et al., J. Biol. Chem., 990, 265:16020-16024). A desaturase capable of introducing six double bonds into phytoene would lead to the production of the fully conjugated carotenoid 3,4,3′,4′-tetradehydrolycopene. The phytoene desaturase from Erwinia uredovorahas been shown to synthesize only trace amounts of 3,4,3′,4′-tetradehydrolycopene under certain conditions (Fraser et al., J. Biol. Chem., 1992, 267:19891-19895).

Starting from neurosporene in Rhodobacter or lycopene in other photosynthetic bacteria, diverse acyclic carotenoids are synthesized by desaturation, hydroxylation and methylation. Erwinia synthesizes cyclic carotenoids from lycopene. These modifying enzymes show a high degree of promiscuity that allows them to act equally well on neurosporene and lycopene in engineered pathways (Ausich, R. L., Pure Appl. Chem., 1994, 66:1057-1062; Hunter et al., J. Bacteriol., 1994, 176:3692-3697; Takaichi et al., Euro. J. Biochem., 1996, 241:291-296; and Albrecht et al., J. Biotechnol., 1997, 58:177-185). It is therefore likely that carotenoids with a further extended chromophore, such as 3,4-didehydrolycopene or 3,4, 3′4′-tetradehydrolycopene, would also be modified by these enzymes or their variants, leading to the production of novel carotenoids.

Bacterial lycopene cyclases usually introduce β-ionone rings at both ends of lycopene to produce β, β-carotene (Cunningham et al., Plant Cell, 1996, 8:1613-1626) (FIG. 1). However, when neurosporene is produced by a three-step desaturase from Rhodobacter or ξ-carotene is produced by a two-step desaturase from Synechococcus sp. in an engineered pathway, the cyclase is capable of cyclizing not only the Ψ end group (as in lycopene and at one end of neurosporene) to the β end group, but also the 7,8-dihydro-Ψ end group (as at one end of neurosporene and in ξ-carotene) to the 7,8-dihydro-β end group (Takaichi, supra, 1996) (see FIG. 1 for carotenoid structures). Synthesis of the respective monocyclic intermediates demonstrates that he enzyme acts on the two ends separately. The proposed reaction mechanism for cyclization involves only the double bonds C1-C2 (C1′-C2′) and C5-C6 (C5′-C6′), which agrees with the observed broad substrate specificity (Hugeney et al., Plant J., 1995, 8:417-424).

Carotenoid biosynthesis in a non-carotenogenic microorganism such as E. coli requires extension of the general terpenoid pathway with the genes for geranylgeranyldiphosphate (GGDP) synthase (crtB) and phytoene synthase (crtE) for the production of the first C₄₀ carotenoid phytoene (FIG. 1). Subsequent desaturation by phytoene desaturase (crtI) and further modifications catalyzed by, e.g., cyclases, hydroxylases, and ketolases result in the production of different carotenoids (Britton, In: Carotenoids: Biosynthesis and Metabolism, Vol. 3, Carotenoids, G. Britton, Ed. Basel: Birkhäuser Verlag, 1998, 13-147).

In the context of a biosynthetic pathway in E. coli, variant enzyme libraries can be created by co-transformation with two plasmids that together are stably propagated. Genes that produce the carotenoid precursors that serve as substrates for the target enzyme are cloned into an appropriate plasmid, such as a pACYC184-derived plasmid. Genes for the enzymes subjected to evolution in vitro are cloned into a different plasmid, such as a pUC19-derived plasmid. All enzymes are preferably individually expressed under the control of a lac-promoter followed by an optimized Shine-Dalgarno sequence, although operon control is also possible.

In a specific embodiment, starting with recombinant E. coli cells expressing GGPP-synthase (crtE) and phytoene synthase (crtB), and hence producing the first C40 carotenoid phytoene, different biosynthetic genes are evolved by random mutagenesis and/or gene shuffling and introduced to this pathway. Enzyme variants leading to the production of novel carotenoids can be combined in a modular way, resulting in additional novel pathways.

For the success of this approach, modular vectors are constructed allowing for the expression of several biosynthetic genes. Most important, high-throughput screening methods have been developed for the identification of recombinants producing novel carotenoids.

Specific gene modifications include:

directed evolution of phytoene desaturase (crtI) for the synthesis of 3,4, 3′,4′-tetradehydrolycopene;

directed evolution of spheroidene-monoxygenases (crtA) and β-C4-ketolase (crtO) for the synthesis of novel, non-cyclic xanthophylls;

directed evolution of lycopene-cyclases (crtY) and β-C4-ketolase (crtO) for the production of novel, cyclic/aromatic xanthophylls;

directed evolution of lycopene-cyclase (crtY) for the production of ε,ε-carotene instead of β,β-carotene;

directed evolution of β-carotene desaturase crtU from Streptomyces griseus for the production of novel aromatic cyclic C40 carotenoids; and

production of novel C30-carotenoids by expression of dehydrosqualene synthase (crtM) and dehydrosqalene desaturase (crtN) from Staphylococcus aureus for 4,4′-diaponeurosporene synthesis in E. coli and a) directed evolution of crtN for the production of diapolycopene, b) directed evolution of crtY (β-carotene cyclase) for cyclization of diaponeurosporene and diapolycopene, and c) adaptation of further enzymes, like β-C4-oxygenases (ketolases, crtW, crtO), β-carotene hydroxylases crtZ, carotene desaturase crtU, and spheroidene-monooxygenase crtA, to modify these diapo-carotenoids.

Optimization of microbial production levels of novel carotenoids and carotenoids in general can be achieved by:

(1) optimizing protein expression levels for maximal production by in vitro mutagenesis of target genes and/or classical regulation of gene expression;

(2) synthesis of water soluble carotenoids by directed evolution of the hydroxylating genes neurosporene dehydrogenase (crtC) and β-carotene hydroxylase (crtZ) as well as the glycosylating enzyme zeaxanthin glucosidase (crtX);

(3) implementing the evolved pathways in microbial hosts like S. cerevisiae, C. utili, or Rhodobacter defect mutants with higher production capacities for hydrophobic carotenoids due to larger membrane storage capacities.

(4) biochemical characterization of novel enzyme variants and pathways.

Construction of Carotenoid Biosynthetic Pathways in E. coli

The following describes a specific embodiment of the invention: creation of carotenoid biosynthetic pathways in E. coli. Modification of these strategies by standard molecular biological techniques adapts them for other microorganisms. Thus, the general techniques for directed evolution applied to specific systems described throughout this application permits creation of carotenoid biosynthetic pathways in other microorganisms. The invention is not limited to these disclosed embodiments, which are exemplified infra.

Furthermore, once a biosynthetic pathway is created in one microorganism, such as E. coli, the pathway can be transferred to a different host system, such as S. cerevisiae or a plant cell.

Methods for directed evolution more preferably include gene shuffling or error-prone PCR, depending on the gene to be evolved. To identify novel carotenoids that demonstrate only small differences in their absorption properties, additional methods, including LC-MS, TLC, and screening of microtiter plates using a plate reader, can be used.

Modular Expression Vectors. In a specific embodiment, for the expression of all these different biosynthetic genes in E. coli, two modified expression vectors based on pUC (lac-promoter, pUCmod) and pKK (tac-promoter, pKKmod) with optimized cloning sites and Shine-Dalgarno sequence, and different promoter strengths, were designed. In addition, a second low-copy number plasmid (pACmod) based on pACYC 184 and compatible to the pUC and pKK-based vectors was designed for complementation in E. coli. While pACmod served as vector for the expression of selected biosynthetic genes (each under the control of its own promoter) for carotenoid production, pUCmod or pKKmod were used for library creation following in vitro mutagenesis or gene shuffling of the target genes.

Assembly of Carotenoid Biosynthesis Pathways. Assembly of the cloned wildtype genes in modular pathways by cloning them into pACmod, pUCmod and pKKmod (metabolic engineering) results in the expected production of various carotenoids and hence verified the functional expression of these genes. Recombinant E. coli cells producing carotenoids turned yellow to orange, depending on the carotenoid produced.

Specific Embodiments of Directed Evolution of Carotenoid Synthases

References herein to genes or gene products in this section by abbreviation are provided for convenience. Any gene having that function can be substituted for a specifically recited gene.

Directed evolution of phytoene-desaturase. In one embodiment, the invention provides for evolving desaturase that introduces six double bonds instead of four into phytoene and thus synthesizes the fully conjugated carotenoid 3,4, 3′,4′- tetradehydrolycopene. To this end, desaturase genes (crtI), such as those from Erwinia hericola and Erwinia uredovora can be recombined in vitro by DNA-shuffling and transformed into phytoene-producing recombinant E. coli cells. Visual screening of approximately 10⁴ clones results in several yellow clones and one pink clone clearly distinguishable from the orange clones producing lycopene. Spectrophotometrical analysis of the carotenoids produced by those mutants shows that the yellow clones produce predominantly β-carotene lacking two of the double bonds found in lycopene. The pink clone, however, produces the fully conjugated linear carotenoid 3,4, 3′,4′ tetradehydrolycopene. Sequence analysis and chimera formation between wildtype gene and the mutant desaturase introducing six double bonds identified amino acid substitutions in a surprising location, e.g., in a putative dinucleotide binding-site not previously known to alter enzyme function in this way.

Complementation of wildtype and mutant desaturase with crtA and crtY. Both wildtype desaturase, synthesizing lycopene, and mutant desaturase, synthesizing 3,4, 3′,4′ tetradehydrolycopene, are each cloned into a suitable plasmid, such as pACmod, along with crtB and crtE necessary for phytoene production if production is to occur in a no carotogenic microorganism. Complementation of lycopene- and 3,4,3′4′ tetradehydrolycopene-, respectively, producing cells with either spheroidene-monooxygenases (crtA), e.g., from Rhodobacter or lycopene cyclases (crtY), e.g., from Erwinia, leads to the formation of acyclic xanthophylls and cyclic carotenoids. Spectrophotometrical analysis of cell extracts indicates, at least in the case of xanthophyll formation, that 3,4,3′,4′ tetradehydrolycopene is converted by crtA to yield different xanthophylls than those produced in cells harboring the wildtype desaturase gene. This is also reflected by the dark, orange-red color of xanthophyll-producing cells harboring the mutant desaturase gene. Carotenoid extracts of lycopene- and 3,4,3′4′ tetradehydrolycopene producing cells complemented with crtA can be analyzed by HPLC equipped with a photodiode-array or mass detector, and/or by NMR. E. coli cells expressing the mutant desaturase along with the lycopene cyclase synthesize exclusively β,β-carotene. In cells expressing the wildtype desaturase along with the lycopene cyclase, β-zeacarotene and β,β-carotene as the cyclization products of neurosporene and lycopene, respectively, accumulate.

Directed evolution of crtA and crtY. In order to improve xanthophyll formation and to produce different xanthophylls by oxygenation of 3,4,3′,4′ tetradehydrolycopen, both crtA genes from Rhodobacter or other microorganisms can be shuffled. The library thus created is screened for promising variants. In parallel, crtY genes of Erwinia or other microorganisms coding for lycopene cyclases shuffled and the library is screened for cyclase variants exhibiting the ability to cyclize 3,4-didehydrolycopene and/or 3,4,3′,4′-tetradehydrolycopene.

Directed evolution of βC4-ketolase (crtO) for the production of novel acyclic xanthophylls. The β-C4-ketoalse (crtO), e.g., from Synechocystes sp., catalyzes the oxygenation of unsaturated C-atoms and hence, can be used for the introduction of aldehyde groups into acyclic carotenoids. The natural substrate for crtO is a cyclic carotenoid, β-carotene. In contrast to other β-carotene ketolases from Agrobacterium and Haematococcus, though, crtO is homologous to microbial phytoene-desaturase. Thus, adaptation of crtO to accept acyclic carotenoids by directed evolution is likely. In a specific embodiment, using 3,4,3,′,4′ tetradehydrolycopene- or lycopene-producing recombinant E. colicells, novel crtO variants introducing aldehyde functions into these substrates are evolved by error-prone PCR and subsequent complementation. Novel variants are screened visually or spectrophotometrically in microtiter plates.

Variants of crtO introducing aldehyde functions into 3,4,3,′,4′ tetradehydrolycopeneor lycopene can be transformed into recombinant host cells harboring crtA variants introducing keto-groups into 3,4,3,′,4′ tetradehydrolycopene or lycopene. Hence, synthesis of xanthophylls with both aldehyde- and keto-functions is possible.

Directed evolution of βC4-ketolase for the production of novel cyclic carotenoids. Novel enzyme variants of crtO are evolved which (i) not only introduce a keto-group at position C4 of β-carotene (echinenone), but also at position C4′ (canthaxanthin) and (ii) introduce keto-groups at positions other than C4 or C4′, possibly resulting in ring rearrangements. Following complementation of β-carotene producing cells with a library of mutated crtO, novel enzyme variants are again identified by a bathochromic shift of the absorption maximum due to the introduction of oxo-groups. For example, while β-carotene is orange, echinenone is orange-red and canthaxanthin is red.

Directed evolution of lycopene cyclase (crtY) for the production of novel cyclic xanthophylls and ε,ε-carotene. In addition to evolving a lycopene cyclase which accepts 3,4,3,′,4′ tetradehydrolycopene as substrate and thus produces the orange-red 3,4,3,′,4′ tetradehydro-ε, ε-carotene, cyclase variants able to accept acyclic xanthophylls, produced by variant crtA and crtO, are evolved. Since cyclization results in a hypsochromic absorption shift and in a loss of spectral fine structure, screening for novel enzyme variants can again be based on altered absorption properties of either host cell clones or cell extracts. Although cyclization of tetrahydrolycopene might be very difficult, the strategies provided here for directed evolution provide the greatest assurance of success.

A second approach to evolve novel lycopene cyclase variants permits production of ε,ε-carotene. Because of the homology between bacterial and plant β-cyclases with plant ε-cyclase and capsanthin-capsorubin-synthase, it is likely that directed evolution of bacterial β-cyclase results in variants with symmetrical or asymmetrical ε-cyclase activity. The synthesis of cyclic carotenoids or xanthophylls with two ε-rings is of especially high interest. Plant β-cyclase and ε-cylcase synthesize together mainly α-carotene (β,ε-carotene), while ε-carotene (ε,ε-carotene) is produced only in very small amounts. Since cyclization isomers of carotene show only small differences in absorption spectra, screening can be based on a microtiter plate screen where absorption of carotenoid extracts can be measured at multiple wavelengths.

Optimization of Carotenoid Production by Metabolic Engineering

In order to gain maximal flux of carotenoids in a desired way through all of the assembled pathways, control and optimization of expression levels and enzyme activities are of major importance for any of the above directed evolution strategies. For instance, directed evolution of cyclases which accept 3,4-didehydrolycopene or 3,4,3′4′ tetradehydrolycopene as substrate requires sufficient production of this carotenoid in E. coli. Otherwise the cyclase would preferentially accept lycopene, since (i) desaturation is a sequential reaction and (ii) a novel variant will most likely have at first only a relatively low affinity for the new substrate 3,4-didehydrolycopene or 3,4,3′,4′ tetradehydrolycopene. Expression levels and enzyme activities can either be increased by choosing an appropriate promoter or by subjecting biosynthetic gene variants to further rounds of random mutagenesis.

A different approach to increase the general carotenoid storage capacity of E. coli is to produce more water-soluble carotenoids, which may increase the bioavailability of carotenoids in medical applications. For example, neurosporene hydroxylase (crtC) can be adapted to novel acyclic xanthophylls or carotenoids derived from preceding evolution rounds. Similarly, β-carotene hydroxylase (crtZ) and zeaxanthin glucosidase (crtX) can be evolved to accept novel cyclic carotenoids as substrates, also derived from preceding evolution rounds. Since, hydroxyl and glucosyl groups do not contribute to the chromophore of carotenoids, screening can either rely on increased production levels by the action of variants of these enzymes or on HPLC-MS screening.

Most carotenogenic genes and gene clusters have been cloned and expressed in the genetically easy to manipulate non-carotenogenic host E. coli. However, E. colihas only a limited supply of the common isoprenoid precursors IPP, DMAPP, and GGDP needed for carotenoid biosynthesis. The production levels of carotenoids E. coli are therefore low (10 -500 1μg⁻¹ cell dry weight) compared to commercially employed carotenogenic microbial strains like Dunalliella, Haematococcus and Xanthophyllomyces dendrorhous (fomerly Phaffia rhodozyma), where production levels of up to 50 mg g⁻¹ cell dry weight are obtained (Johnson and Schroeder, Adv. Biochem. Engineering and Biotech., 1995, 53:119). Efforts to increase the isoprenoid central flux in E. coli (reviewed in Misawa and Shimada., J. Biotech., 1998, 59:169) have been directed at increasing the production of IPP, produced through the mevalonate-independent pathway recently discovered in eubacteria, plants, and algae (see, Britton, G., In: Carotenoids: Biosynthesis and Metabolism, Vol. 3, Carotenoids, G. Britton, Ed. Basel: Birkhäuser Verlag, 1998, 13-147, and references therein), and of GGDP from IDP. Overexpression of the IDP isomerase (idi) that catalyzes the isomerisation of IDP to DMAP, and an archaebacterial GGDP synthase (gps) that converts IDP and DMAPD directly to GDP, resulted in an approximately 50-fold increase of astaxanthin production (Wang et al., Biotech. Bioengineering, 1999, 1:235). Similarly, production levels of about 1500 μg g⁻¹ dry weight β-carotene and zeaxanthin could be obtained in E. coli by overexpression of the 1 -deoxy-D-xylose 5-phosphate synthase (dxs) involved in IPP synthesis and idi (Albrecht et al., Biotech. Letter, 1999, 21:791). Further increase of IDP synthesis by coexpression of idi, dxs and a 1-deoxy-D-xylose 5-phosphate reductase (dxr) was toxic for E. coli possibly due to overloading the membranes with carotenoids.

Other suitable bacterial hosts include, but are by no means limited to, Synechocystis sp., E. niobilis, Z. mobilis, and Agrobacterium tumefaciens, as well as modified variants of carotogenic bacteria.

Thus, an alternative option for the improvement of carotenoid yields is the use of recombinant Rhodobacter strains, deficient in the production of several carotenoids (Komori et al., Biochem., 1998, 37:8987). The photosynthetic purple bacterium Rhodobacter would naturally be a good host for carotenoid production due to its large membrane storage capacities. Expression cassettes used for E. coli can be transferred onto a shuttle vector and expressed under the control of the same promoters, lac or tac, used in E. coli.

Yeasts (e.g., S. cerevisiae, Candida utilis, X dendrorhous, which are non-limiting examples) are capable of accumulating large quantities of the isoprenoid derivative ergosterol. Ergosterol biosynthesis has been successfully diverted for the production of carotenoids in the non-carotenogenic yeasts S. cerevisiae and Candida utilis (reviewed in Misawa and Shimada, supra). Recently, overexpression of the HMG-CoA reductase (involved in the mevalonate synthesis pathway) and blockage of ergosterol synthesis by disruption of the ERG9 gene encoding squalene synthase yielded a lycopene overproducing C. utilis strain (7.8 mg g-1 dry weight) with commercial potential after introducing of the carotenogenic genes crtE, crtB, and crtI (Shimada et al., Appl. and Environmental Microbiol., 1998, 64:2676).

Production levels of carotenoids in yeasts are generally significantly higher than in E. coli (Misawa and Shimada, supra, 1998). For example, the yeast X. dendrorhous (Phafffia rhodozyma) is capable of producing up to 50 mg per gram of cells, while carotenoid production levels in E. coli range from 0.1-1.5 mg/g cells. The increased carotenoid yields in recombinant yeasts are mainly attributed to their larger membrane storage capacities for the hydrophobic carotenoids. Hence, selected evolved pathways for the production of novel carotenoids can be transferred into S. cerevisiae. Different vectors, 2μm-based vectors and integration vectors, and different promoters for the optimization of gene copy numbers and expression levels in yeast are used.

In addition to yeasts, the invention permits manipulation of fungi (e.g., Phycomyces blakesleeanus) and algae (e.g., H. pluvalis) to create carotenoid biosynthetic pathways of the invention.

It should be noted, that, apart from engineering microbial carotenoid biosynthesis, there have been recent accomplishments in manipulating carotenoid biosynthesis in transgenic plants as reviewed (Hirschberg, Curr. Op. Biotech., 1999, 10:186; Mann et al., Nature Biotechnol 2000;18:888-892; Roemer et al., Nature Biotechnol. 2000;18:666-669; Ye et al., Science 2000; 287:303-305). The pathways of this invention created in microorganisms, such as E. coli, can be transferred to plants, such as but by no means limited to Arabidopsis thaliana.

Synthesis of the carotenoid 3,4,3′,4′-tetradehydrolycopene in E. coli by directed evolution of the crtI enzyme already demonstrates the feasibility of rational assembly of biosynthetic gene and directed evolution of key enzymes for the production of new metabolites. Furthermore, a library of shuffled lycopene cyclases (crtY) in E. coli yielded a microorganism capable of synthesis of torulene, a product never before known to be produced in any bacteria, via a very different pathway from that employed in yeast that naturally synthesize this metabolite. Preliminary results also indicate that 3,4,3′,4′-tetrahydrolycopene serves as a substrate of the monooxygenases crtA. Hence, biosynthesis enzymes may have broader substrate specificities than are naturally seen in an endogenous pathway, indicating that not every gene in a tailor-made pathway needs to be highly adapted to a novel substrate and thus speeding up the process for the synthesis of new metabolites.

### Development of Carotenoid Analytical and Screening Methods

Since carotenoids exhibit specific absorption properties depending on their chromophore, novel carotenoids can be distinguished by their altered light absorption properties when the enzymatic modifications affect the chromophore. In order to facilitate screening based on altered spectrophotometrical properties for synthesis of novel carotenoids, biosynthesis enzymes are chosen for directed evolution which affect the chromophore by, e.g., desaturation, oxygenation or cyclization. Detailed methods for carotenoid analysis are found in Britton et al., In: Carotenoids: Volume 1A: Isolation and Analysis, Basel: Birkhäuser Verlag; and Britton et al., In: Carotenoids: Volume 1B: Isolation and Analysis, Basel:Birkhäuser Verlag.

In a specific embodiment, two different methods for screening of recombinant E. coli libraries for novel carotenoid production were developed. The first screen is a simple plate-screen based on a filter transfer for visualization of carotenoid producing clones. The second screen is a microtiter plate screen involving organic solvent extractions of carotenoids, followed by absorption measurements at multiple wavelengths with a plate reader.

### Polyketides

### General

Polyketides are natural products occurring in a wide variety of organisms, particularly abundant in the actinomycetes, a class of mycelial bacteria. The structurally diversity among the polyketides has allowed for extensive use in various medical areas. Examples of medically important polyketides include tetracyclines and erythromycin (antibiotics); daunomycin (cytostatic drug), and rapamycin (immunosuppressant).

### Polyketide Biosynthetic Genes

Polyketide synthases are multifunctional enzymes which catalyze the biosynthesis of polyketides through repeated (decarboxylative) Claisen condensations between acylthioesters, e.g., acetyl, malonyl, methylmalonyl, or propionyl. After each condensation, they introduce structural variability into the product by catalyzing all, part, or none of a reductive cycle comprising a ketoreduction, dehydration, and enoylreduction on the, β-ketogroup of the growing polyketide chain. Polyketide synthases incorporate enormous structural diversity into their products, in addition to varying the condensation cycle, by controlling the overall chain length, choice of primer and extender units and, particularly in the case of aromatic polyketides, regiospecific cyclizations of the nascent polyketide chain. After the carbon chain has grown to a length characteristic of each specific product, it is released from the synthase by thiolysis or acyltransfer. Thus, polyketide synthases consist of families of active sites which work together to produce a given polyketide. It is the controlled variation in chain length, choice of chain-building units, and the reductive cycle, genetically programmed into each enzyme unit, that contributes to the variation among naturally occurring polyketides.

There are two general classes of polyketide synthases; Type I, “modular” enzymes PKSs including assemblies of several large multifunctional proteins carrying, between them, a set of separate active sites for each step of carbon chain assembly and modification (Cortes, J. et al. Nature 1990;348:176, Donadio, S., et al., Science 1991;252:675, MacNeil, D. J., et al., Gene 1992; 115:119), and Type II, non-modular, synthases for aromatic compounds.

Streptomyces is an actinomycete producing various aromatic polyketides. For instance, Streptomyces coelicolor produces the blue-pigmented polyketide, actinorhodin. The actinorhodin gene cluster has been cloned (Malpartida, F., and Hopwood, D. A., Nature 1984;309:462, Malpartida, F. and Hopwood, D. A. Mol. Gen. Genet. 1986;205:66) and sequenced (Hallam, S. E., et al., Gene 1988;74:305; Fernandez-Moreno, M. A., et al., Cell 1991;66:769, Caballero, J. et al., Mol. Gen. Genet. 1991 ;230:401). Examples of enzymes involved in polyketide biosynthesis are listed below in Table 5.

TABLE 5
Selection of Sequences Encoding Polyketide Biosynthesis Enzymes

| ENZYME/PATHWAY | ORGANISM | ACCESSION NO. |
|---|---|---|
| nogalamycin biosynthesis | Streptomyces nogalater | AJ224512 |
| RdmC, RdmD, and RdmE | Streptomyces purpurascens | U10405 |
| transposase | Sorangium cellutosum | AF217189 |
| type II thioesterase | Streptomyces venezuelae | AF193868 |
| type II thioesterase | Streptomyces narbonensis | AF193867 |
| EryA | Saccharopolyspora erytbraea | M63676 |
| griseus nonactin biosynthesis | Streptomyces griseus | AF074603 |
| glycosyltransferase, methyltransferase, | Streptomyces argillaceus | AF077869 |
| and oxygenase | | |
| deoxyhexose reductase | Streptomyces noursei | AF071520 |
| TD-glucose dehydratase | Streptomyces noursei | AF071519 |
| epothilone biosynthesis | Sorangium cellulosum | AF210843 |
| polyketide synthase | Aspergillus fumigatus | AF025541 |
| ketoreductase, glycosyl transferase, | Streptomyces nogalater | AF187532 |
| nogalonic acid methyl ester cyclase, | | |
| dTDP-glucose-4,6-dyhdratase, dTDP- | | |
| 4-dehydrorhamnose reductase, | | |
| polyketide cyclase, amino methylase, | | |
| and dTDP-glucose synthase | | |
| polyketide synthase | Gibberella fujikuroi | AF155773 |
| lovastatin nonaketide synthase | Aspergillus terreus | AF151722 |
| mithramycin biosynthetis | Streptomyces argillaceus | AJ007932 |
| jadomycin polyketide synthase | Streptomyces venezuelae | AF126429 |
| clyclase, jadomycin polyketide | | |
| ketosynthase, ketoreductase, | | |
| bifunctional cyclase/dehydrase | | |
| lovastatin biosynthesis | Aspergillus terreus | AF141925 |
| lovastatin biosynthesis | Aspergillus terreus | AF141924 |
| 2,4-diacetyiphioroglucinol | Pseudomonas | U41818 |
| biosynthesis | | |
| pimS1 | Streptomyces natalensis | AJ132222 |
| pimS0 | Streptomyces natalensis | AJ132221 |
| cytoebrome P450 monooxygenase | Myxococcus xanthus | AJ232955 |
| pyoluteorin biosynthesis | Pseudomonas fluorescens | AF081920 |
| pksP | Aspergillus fimigatus | Y17317 |
| granaticin biosynthesis | Streptomyces violaceoruber | AJ011500 |
| tal | Myxococcus xanthus | AJ006977 |
| eryG and eryA, eryBII, eryCIII, eryCII | Saccharopolyspora erythraea | Y14332 |
| chalcone synthase | Pinus strobes | AJ004800 |
| daunorubicin, doxorubicin, and | Streptomyces peucetius | U77891 |
| baumycin biosunthesis | | |
| coronafacic acid biosynthesis | Pseudomonas syringac | AF098795 |
| sterigmatocystin biosynthesis | Emericella nidulans | U34740 |
| polyketide synthase | Aspergillus parasiticus | L42766 |
| polyketide synthase | Aspergillus parasiticus | L42765 |
| glycinca oxidoreductase | Pseudomonas syringac | AF061506 |
| polyketide cyclase | Streptomyces pseucetius | AF048833 |
| polyketide synthase | Aspergillus parasiticus | U52151 |
| frenolicin biosynthesis | Streptomyces roseofulvus | AF058302 |
| rifamycin biosynthesis | Amycolatopsis mediterranei | AF040570 |
| polyketide synthase | Colletotrichum lagenarium | D83643 |
| cutR, cutS | S.lividans | X58793 |
| DNA for mtmQ, mtmX, mtmP, | S.argillaceus | X89899 |
| mtmK, mtmS and mtmTl | | |
| polyketide synthase | A.parasiticus | Z47198 |
| fabD, fabH, fabC, fabB, and ORF5 | Streptomyces glaucescens | L43074 |
| urdE,F,A,B,C,D | S.fradiae | X87093 |
| polyketide synthase | Emericella nidulans | L39121 |
| polyketide synthase | Streptomyces antibioticus | L09654 |
| polyketide synthase | Streptomyces roseofulvus | L263 38 |
| polyketide synthase type I | Streptomyces noursei | AF071523 |
| O-methyhransferase | Streptomyces noursei | AF071517 |
| P450 hydroxylase | Streptomyces noursei | AF071516 |
| glycosyltransferase | Streptomyces noursei | AF071514 |
| avermectin biosynthesis | Streptomyces avermitilis | AB032523 |
| polyketide synthase | Streptomyces venezuelae | AF079138 |
| pikCD operon | Streptomyces venezuelae | AF079139 |
| desosamine biosynthesis | Streptomyces venezuelae | AF079762 |
| macrolide antibiotics 3-O- | Streptomyces thermotolerans | D30759 |
| acyltransferase, carbomycin 4-O- | | |
| metyltranferase | | |
| polyketide synthase, hydroxylase | Streptomyces sp. | Y10438 |
| polyketide synthase | Sorangium cellulosum | U24241 |
| valine dehydrogenase | Streptomyces fradiae | L33872 |
| macrocyn-O-methyltransferase | S .fradiae | J03008 |
| acyltranferase O-methyltranferase | Streptomyces mycarofaciens | M93958 |

Literature describing the structure and/or function of these genes can be found in Ylihonko K, et al., Mol Gen Genet May 23, 1996;251(2):113-20; Torkkell S, et al., Mol Gen Genet Sep. 1997;256(2):203-9; Ylihonko K, et al., Mol Gen Genet May 23, 1996;251(2):113-20; Torkkell S, et al., Mol Gen Genet September 1997;256(2):203-9; Ikeda H, et al., Proc Natl Acad Sci U S A Aug. 17,1999; 96(17):9509-14; MacNeil D J, et al., Gene Jun. 15, 1992;115(1-2):119-25; Ikeda H, et al., Gene Jan. 12, 1998;206(2):175-80; Denoya C D, et al., J Bacteriol June 1995;177(12):3504-11; Ikeda H, et al., Gene Jan. 12, 1998;206(2):175-80; Schwecke T, et al.; Proc Natl Acad Sci U S A Aug. 15, 1995; 92(17):7839-43; Molnar I, et al., Gene Feb. 22, 1996;169(1):1-7; Aparicio J F, et al., Gene Feb. 22. 1996; 169(1):9-16; Ruan X, et al., Gene Dec. 5, 1997;203(1):1-9; Ruan X, et al., Gene Dec 5, 1997; 203(1):1-9; Aparicio J F, et al., J Biol Chem Apr. 9, 1999;274(15):10133-9; Motamedi H, et al., Eur J Biochem Feb. 15, 1997;244(1):74-80; Motamedi H, et al., Eur J Biochem Sep. 15, 1998; 256(3):528-34; Motamedi H, et al., J Bacteriol September 1996;178(17):5243-8; Bergh S, et al., Biotechnol Appl Biochem Feb. 15, 1992;(1):80-9; Lombo F, et al., Gene Jun. 12, 1996;172(1):87-91; Prado L, et al., Mol Gen Genet March 1999;261(2):216-25; Blanco Gm, et al., Mol Gen Genet Jan. 2000; 262(6):991-1000; Fernandez E, et al., J Bacteriol September 1998;180(18):4929-37; Lombo F, et al., J Bacteriol May 1997;179(10):3354-7; Lozano M J, et al., J Biol Chem Feb. 4, 2000;275(5):3065-74; Grimm A, et al., Gene Dec 30, 1994;151(1-2):1-10; Rangaswamy V, et al.,: Proc Natl Acad Sci U S A Dec. 22, 1998;95(26):15469-74; Penfold C N, et al., Gene Dec. 12, 1996;183(1-2):167-73; Rangaswamy V, et al., J Bacteriol July 1998;180(13):3330-8; Decker H, et al., J Bacteriol November 1995; 177(21):6126-36; Decker H, et al., J Bacteriol November 1995;177(21):6126-36; Faust B, et al., Microbiology January 2000;146 (Pt 1):147-54; Bibb M J, et al., Gene May 3, 1994;142(1):31-9; Ichinose K, et al., Chem Biol Nov. 5, 1998;(11):647-59; Sherman D H, et al., EMBO J Sep. 8, 1989;(9):2717-25; Bechthold A, et al., Mol Gen Genet Sep. 20, 1995;248(5):610-20; Schupp T, et al., J Bacteriol July 1995; 177(13):3673-9; Xue Y, et al., Proc Natl Acad Sci U S A Oct. 13, 1998;95(21):12111-6; Bevitt D J, et al., Eur J Biochem Feb. 15, 1992;204(1):39-49; Cortes J, et al., Nature Nov. 8, 1990; 348(6297):176-8; Ye J, et al., J Bacteriol Oct. 1994;176(20):6270-80; Rajgarhia V B, et al., J Bacteriol April 1997;179(8):2690-6; Filippini S, et al., Microbiology Apr. 1995;141 (Pt 4):1007-16; Dickens M L, et al., J Bacteriol Feb. 1995;177(3):536-43; Filippini S, et al., Microbiology Apr. 1995; 141 (Pt 4):1007-16; Krugel H, et al., Mol Gen Genet Oct. 1993;241(1-2):193-202; Kim E S. et al., Gene Apr. 8, 1994;141(1):141-2; Bibb M J, et al., EMBO J Sep. 8, 1989;(9):2727-36; Summers R G, et al., J Bacteriol March 1992;174(6):1810-20; Bate N, et al., Microbiology Jan. 2000;146 (Pt 1):139-46; Fouces R, et al., Microbiology April 1999;145 (Pt 4):855-68; Arisawa A, et al., Appl Environ Microbiol July 1994;60(7):2657-60; Arisawa A, et al., Biosci Biotechnol Biochem Dec. 1993; 57(12):2020-5; Merson-Davies L A, et al., Mol Microbiol Jul. 13, 1994;(2):349-55; Chang P K, et al., Mol Gen Genet Aug. 21, 1995;248(3):270-7; Feng G H, et al., J Bacteriol Nov. 1995; 177(21):6246-54; Mao Y, et al., Chem Biol Apr. 6, 1999;(4):251-63; Mao Y, et al., J Bacteriol April 1999;181(7):2199-208; Olano C, et al., Mol Gen Genet August 1998;259(3):299-308; Hu Z, et al., Mol Microbiol Oct. 14, 1994;(1):163-72; Chen S, et al., Eur J Biochem April 1999;261(1):98-107; Niemi J, et al., J Bacteriol May 1995;177(10):2942-5; Kantola J, et al., Microbiology January 2000;146 (Pt 1):155-63; Brunker P, et al., Gene Feb. 18, 1999;227(2):125-35; Schupp T, et al., FEMS Microbiol Lett Feb. 15, 1998;159(2):201-7; Piecq M, et al., DNA Seq 1994;4(4):219-29; Yu J H, et al., J Bacteriol August 1995;177(16):4792-800; Han L, et al., Microbiology December 1994;140 (Pt 12):3379-89; Gould S J, et al.,: J Antibiot (Tokyo) Jan. 1998;51(1):52-7; Xue Y, et al., Gene Mar. 7, 2000; 245(1):203-11; Silakowski B, et al., J Biol Chem Dec. 24, 1999;274(52):37391-9; Beye S. et al., Biochim Biophys Acta May 14, 1999;1445(2):185-95; Xue Y, et al., Chem Biol Nov.5, 1998; (11):661-7; Graziani E I, et al., Bioorg Med Chem Lett Nov. 17, 1998;8(22):3117-20; Xue Y, et al., Gene March 7, 2000;245(1):203-11; Hara O, et al., J Bacteriol August 1992;174(15): 5141-4; Wang Y G, et al., Chin J Biotechnol 1989;5(4):191-201; Zotchev S, et al., Microbiology March 2000;146 (Pt 3):611-9; Walczak R J, et al., FEMS Microbiol Lett Feb. 1, 2000;183(1):171-5; Kakavas S J, et al., J Bacteriol December 1997;179(23):7515-22; Takano Y, et al., Mol Gen Genet Nov. 15, 1995; 249(2):162-7;Funa N, et al., Nature Aug. 26, 1999;400(6747):897-9; Paitan Y, et al., J Mol Biol Feb. 19, 1999;286(2):465-74; Paitan Y, et al., Microbiology November 1999;145 (Pt 11):3059-67; Nowak-Thompson B, et al., Gene Dec. 19, 1997;204(1-2):17-24; Dairi T, et al., Biosci Biotechnol Biochem September 1997;61(9):1445-53; Yu T W, et al., J Bacteriol May 1994;176(9):2627-34; Proctor R H, et al., Fungal Genet Biol Jun. 27, 1999;(1):100-12; Tsukamoto N, et al., J Bacteriol April 1994; 176(8):2473-5; Tsukamoto N, et al., J Antibiot (Tokyo) August 1992;45(8):1286-94; Dickens M L, et al., J Bacteriol Jun. 1996;178(11):3384-8; Fernandez-Moreno M A, et al., J Biol Chem Sep. 25, 1992; 267(27):19278-90; Fujii I, et al., Mol Gen Genet Nov. 27, 1996;253(1-2):1-10; Hida T, et al., Mol Gen Genet Nov. 27, 1995;249(3):274-80.

### Directed evolution of Polyketide Biosynthetic Pathways

The above listed enzymes, as well as other enzymes of potential interest, may be applied in the context of the invention to produce known or novel polyketides in a more efficient manner.

### Flavonoids

### General

Flavonoids are polyphenolic compounds that are ubiquitously present in foods of plant origin such as fruits, vegetables, nuts, seeds, flowers, leaves, bark, tea and wine. Flavonoids are categorised into flavonols (quercetin, kaempherol, myricetin), flavones (apigenin, luteolin), flavanones (catechin, epicatechin), anthocyanidins and isof lavonoids (genistein, daidzein), and include more than 4000 different compounds.

The function of polyphenols in plants are antioxidants (protection from UV light), protection from insects, fungi, viruses and bacteria, visual attention-pollinator attraction, feed repellent and plant hormone controllers. Due to their activity as antioxidants, dietary flavonoids may have a potent antioxidant, anti-inflammatory and/or antiviral capacity in humans, by altering enzyme activities related to cell division, proliferation, platelet aggregation and immune response. Flavonoids have also been investigated for their anticarcinogenic activities. Various flavonoids, most notably the isof lavonoids, are able to bind non-trivially to estrogen receptors and possess estrogenic or antiestrogenic activities.

The basic flavonoid structure allows a multitude of variations in chemical structure. Improvement and modification of the flavonoid biosynthetic pathways according to the invention can therefore, coupled with appropriate screening techniques, yield novel flavonoids of potential interest for both pharmaceutical and other applications.

### Flavonoid Biosynthetic Genes

One biosynthetic pathway leads from chorismic acid via phenylalanine and/or tyrosine to aromatic compounds such as the primary metabolite lignin and numerous secondary metabolites such as alkaloids, flavonoids, and phenolics. (See Metzler D. E., supra). In the synthesis of flavonoids, phenylalanine is converted to trans-cinnamatic acid by L-phenylalanine ammonia-lyase and cinnamoyl-CoA by 4-coumaroyl-CoA synthetase. The latter serves as the starting material for chain elongation on malonyl-CoA by chalcone synthase. The resulting β-polyketone derivative is cyclized via a Claisen condensation, and further processed into chalcones, flavanones and flavones by chalcone isomerase. These in turn can be converted into the yellow flavonol pigments and the red, purple, and blue anthocyanidins. One example of a flavonoid biosynthetic pathway is shown in FIG. 12. A review of flavonoid biosynthetic pathways can be found in Derwick, 1997 (In: Medicinal Natural Products, J. Wiley & Sons, New York) and references therein.

Some non-limiting examples of mRNA sequences encoding for enzymes involved in the flavonoid biosynthetic pathway, which are contemplated for modification according to the invention, are listed in Table 6.

TABLE 6
Selection of Sequences Encoding Flavonoid Biosynthesis Enzymes

| | | ACCESS- |
|---|---|---|
| | | ION |
| ENZYME/PATHWAY | ORGANISM | NO. |
| flavonoid 3′,5′-hydroxylase | Catharanthus roseus | AJ011862 |
| glucose:flavonoid 3-O-glucosyl | Malus domestica | AF117267 |
| transferase | Perilla frutescense | AB002818 |
| | V. vinifera | X75968 |
| chalcone synthase | V. vinifera | X75969 |
| | Parsley | V01538 |
| chalcone isomerase | V. vinifera | X75963 |
| flavanone-3-hydroxylase | Citrus Cinensis | AB011796 |
| | V. Vinifera | X75965 |
| flavonol synthase | Citrus unshiu | AB011795 |
| dihydroflavonol reductase | V. vinifera | X75964 |
| anthocyanidine synthase | Dianthus caryophyllus | U82432 |
| leukoanthocyanidine dioxygenase | V. vinifera | X75966 |
| stilbene synthase | V. vinfera | X76892 |

Literature describing the structure and/or function of these genes can be found in Gong Z, et al., Plant Mol Biol December 1997;35(6):915-27; Yamazaki M, et al., J Biol Chem Mar. 12, 1999; 274(11):7405-11; Gong Z Z, et al., Plant Mol Biol Sep. 1999;41(1):33-44; Sparvoli F, et al., Plant Mol Biol Mar. 24, 1994;(5):743-55; Saito K, et al., Plant J Jan. 17, 1999;(2):181-9; O'Neill S D, et al., Mol Gen Genet November 1990;224(2):279-88; Rosati C, et al., Plant Mol Biol Oct. 1997;35(3):303-11; van Tunen A J, et al., EMBO J May 7, 1988;(5):1257-63; Charrier B, et al., Plant Mol Biol Nov. 29, 1995; (4):773-86; Tanaka Y, et al., Plant Cell Physiol Jul. 1996;37(5):711-6; Feinbaum R L, Mol Cell Biol May 8, 1988;(5):1985-92; Tanaka Y, et al., Plant Cell Physiol Sep. 1995;36(6):1023-31; Beld M, et al., Plant Mol Biol Nov. 13, 1989;(5):491-502; Boss P K, et al., Plant Mol Biol November 1996; 32(3):565-9; McKhann H I, et al., Plant Mol Biol Mar. 24, 1994;(5):767-77; Batschauer A, et al., Plant Mol Biol Feb. 16, 1991;(2):175-85; Melchior F, et al., FEBS Lett Jul. 30, 1990;268(1):17-20; Ford C M, et al., J Biol Chem Apr. 10, 1998;273(15):9224-33; Schroder J, et al., Z Naturforsch [C] January -February 1990;45(1-2):1-8; Grotewold E, et al., Mol Gen Genet January 1994;242(1):1-8.

### Directed Evolution of Flavonoid Biosynthetic Pathways

The above listed enzymes, as well as other enzymes of potential interest, may be applied in the context of the invention to produce known or novel terpenoids in a more efficient manner.

### Development of Flavonoid Analytical and Screening Methods.

Since flavonoids exhibit specific absorption properties depending on their chromophore, novel flavonoids can be distinguished by their altered light absorption properties when the enzymatic modifications affect the chromophore. In order to facilitate screening based on altered spectrophotometrical properties for synthesis of novel flavonoids, biosynthesis enzymes are chosen for directed evolution which affect the chromophore by, e.g., desaturation, oxygenation or cyclization. Other modifications of the flavonoid structures can be detected by, e.g., LC-MS techniques.

### Tetrapyrroles

### General

Tetrapyrroles are major constituents of every living cell, and are involved in electron transport systems and function as prosthetic groups of many enzymes. Due to their importance to all living systems and their intense coloring they have also been named the “pigments of life”. At present, seven different tetrapyrrole classes are known: haems, chlorophylls and corrinoids (e.g. co-enzyme B₁₂) as the well-known and wide-spread enzyme cofactors; bilins, which are linear tetrapyrroles used for light-harvesting in cyanobacteria and algae; sirohaem in sulphite reductase; haem d1 in nitrite reductase/cytochrome oxidase in denitrifying bacteria and coenzyme F₄₃₀ in methanogenic archeae. Cyclic tetrapyrrole derivatives are derived from a common porphorynogen structure in which the four pyrrolic rings are usually linked by methine bridges. Exceptions are the corrinoids where ring D and A are directly linked. All four rings are at various oxidation levels and, depending on the tetrapyrrole class, have various substituents like acetate, propionate, methyl, ethyl or vinyl groups. All substituents occur in the same order on three of the pyrrolic rings but are reversed on the fourth ring D. Metal ions such as iron, magnesium, cobalt and nickel can be complexed by the central nitrogen atoms. The various oxidation levels of the tetrapyrroles, diversity of the ring substituents and complexed metal ions contribute to their many biological functions.

From a biotechnological point of view, porphyrinoids comprises a class of highly valuable chemicals with many applications in chemistry and medicine (Franck, B. and Nonn, A., Angew. Chem. Int. Ed. Engl. 1995;34:1795-1811). Due to their complex structures, chemical synthesis demands cumbersome multi-step synthesis with very low overall yields. Accordingly, commercially synthesized tetrapyrroles are generally very expensive. A recent summary of novel porphyrinoids in chemistry and medicine as well of the major problems in chemical synthesis is given in Frank and Nonn, supra. Thus, development of tetrapyrrole synthetic pathways according to the invention may lead to a more efficient synthesis of various natural tetrapyrrole derivatives and novel derivatives not found in nature, e.g., by molecular pathway breeding in genetically engineered microorganisms, thereby overcoming and/or avoiding some of the problems related to the currently used chemical syntheses.

### Tetrapyrrole Biosynthetic Genes

All tetrapyrroles are derived from a single common macrocycle, uroporphyrinogen III (urógen III). At this point a major branching of the biosynthetic pathways occurs. (See Battersby, A. R., and Leeper, F. J., Top. Curr. Chem. 1998;195:143-193 for comprehensive figures). (See Chadwick, D. J. and Ackrill, K. (eds.), In: Ciba Foundation Symposia 180: The Biosynthesis of the Tetrapyrrole Pigments; Chichester, Wiley 1994; Jahn, D., et al., Naturwissenschaften 1996;83:389-400; Roth, J. R., et al., Annu. Rev. Microbiol. 1996;50: 137-181; Suzuki, J. et al., Annu. Rev. Gen. 1997;31:61-89; Scott, I. A., Phil. Trans. R. Soc. Lond. A. 1998;356:1341-1366; and Battersby, A. R. and Leeper, F. J., Top. Curr. Chem. 1998;195:143-193). Enzymes and genes involved in urógen III synthesis have been identified for many microorganisms, whereas complete biosynthetic pathways at later branching points have only been identified at a molecular level for haem (not haem b and bilins), chlorophylls and recently, co-enzyme B₁₂ biosynthesis. However, some genes of enzymes involved e.g. in bilin synthesis have been cloned. The most complex biosynthetic pathway, though the evolutionary more ancient pathway, is at present co-enzyme B₁₂ biosynthesis where, depending on the microorganism, up to 30 genes are involved.

As in the case of the carotenoids, tetrapyrrole biosynthesis genes have mainly been isolated by complementation of recombinant E. coli. Most microbial biosynthesis genes isolated so far can be functionally expressed in E. coliand, depending on the biosynthetic genes transformed into E. coli, result in coloration and/or intense fluorescence of the E. coli cells (See Chadwick and Ackrill, supra, Fujino, E., et al., J. Bacteriol.1995;177:5169-5175). An overview of porphyrrin biosynthesis, including enzymes, genes, and biochemical reactions can be found at the following World Wide Web address: genome.adjp/kegg/pathway/map/map00860.html.

### Directed Evolution of Tetrapyrrole Biosynthetic Pathways

Analogous to the molecular pathway breeding of carotenoid biosynthetic pathways, novel biosynthetic capacities of tetrapyrroles in E. coli can be explored by assembly of biosynthetic genes from different microbial sources and in vitro mutagenesis of selected enzymes. Because of the reasonably well understood enzyme functions involved in haem and co-enzyme B₁₂ synthesis and their availability from different microbial sources, biosynthetic genes of these two pathways can be used in the initial experiments.

Though many genes involved in tetrapyrrole biosynthesis have been identified from various microbial organisms, including complete pathways for urógen III, haem, chlorophyll and co-enzyme B12 synthesis, and their enzymatic functions characterized, little is known how to employ these genes for tetrapyrrole production in recombinant microorganisms. At present, only vitamin B₁₂ is biotechnological produced on a large scale by genetically engineered Ps. denitrificans (Chadwick, D. J. and Ackrill, K. (eds.): Ciba Foundation Symposia 180: The Biosynthesis of the Tetrapyrrole Pigments. Chichester: Wiley 1994). Thus, apart from cloning of the necessary biosynthesis genes from the respective microorganism, as in the case for the carotenoid biosynthesis, basic overproduction of tetrapyrrole precursors can be established in E. coli. Although E. coli exhibits biosynthetic genes needed for the production of urógen III, sirohaem and haem, these genes and hence the tetrapyrrole co-factors are only produced in small amounts sufficient for cell functions. Accordingly, gene regulation is under strict control of cellular needs.

Hence, in one embodiment, an efficient urógen III biosynthesis in E. coli is established, followed by the biosynthesis of the main intermediates of haem and co-enzyme B₁₂ biosynthesis which shall be modified by novel biosynthetic enzyme variants. Enzyme variants are evolved either by random mutagenesis or, if two genes with sufficient homology are available, by gene shuffling followed by screening of the resulting E. coli library for new biosynthesis properties.

Cloning of biosynthetic genes and construction of expression vectors

In one embodiment, biosynthetic genes necessary for porphyrin overproduction in E. coli are isolated by retro-PCR from genomic DNA of the respective microorganisms based on published nucleotide sequences. The following genes are cloned:

hemA:5-aminolaevulinate(ALA) synthase from Rhodobacter sphaeroides

hemB: ALA dehydratase from E. coli and Bacillus subtilis

hemC: porphobilinogen deaminase from E. coli and Bacillus subtilis

hemD: urógen synthase from Bacillus subtilis

hemE: urógen decarboxylase from E. coli and Bacillus subtilis

hemF: coproporphyrinogen III oxidase from E. coli

HEM14: Protoporphyrinogen IX oxidase from S. cerevisiae

hemH: ferrochelatase from E. coli and Bacillus subtilis

cobA. urógen methyltransferase from Pseudomonas denitrificans

cobI. precorrin-2 methyltransferase from Pseudomonas denitrificans

Construction of modular expression vectors is basically similar as described for directed evolution of carotenoid biosynthesis (See, supra). However, to ensure overproduction of urógen II, the universal precursor for all naturally occurring tetrapyrroles, a third low-copy vector is used. To this end, the vector pFN467 is modified to allow expression of the genes necessary for urógen III synthesis in E. coli.

Mechanistic aspects of the reactions catalyzed by these enzymes are summarized in (Chadwick, D. J. and Ackrill, K. (eds.): Ciba Foundation Symposia 180: The Biosynthesis of the Tetrapyrrole Pigments. Chichester: Wiley 1994).

Urógen III Overproduction in E. coli

In most bacteria, plants and algae 5-aminolevulinic acid (ALA), as the primary precursor for tetrapyrrole biosynthesis, is synthesized by converting glutamyl-tRNA to glutamate-1-semialdehyde and further to ALA (C5 pathway). In contrast, only the α-group of proteobacteria, e.g., Rhodobacter sphaeroides, animals and fungi synthesize ALA in a protein biosynthesis independent way (Shemin pathway). Here, condensation of one molecule succinyl-CoA and glycine into ALA is catalyzed by one enzyme, the ALA synthase (hem A). Since E. coli uses the C5 pathway for ALA synthesis, ALA synthase (hemA) from R. sphaeroides is employed for ALA synthesis in order to allow for protein biosynthesis independent production, which might become crucial during overexpression of the many biosynthetic genes in E. coli.

Subsequent enzymes in the pathway for urógen III synthesis are hemB and hemC. These genes are cloned either from Bacillus subtilis or E. coli. Both genes for hemB and hemC can be assembled in pathways and investigated for functional expression. The best enzymes are chosen for further work. The last gene necessary for urógen synthesis is hemD, coding for urógen III synthase. Since the E. coli urógen III synthase (cysG) is a natural fusion protein, functioning also as a sirohaem chelatase, only hemD from B. subtillis are used. All these genes are cloned either in a pKK or pUC derived vector and thus, expressed under the control of a tac or lac promoter, respectively. Genes for hemA, hemB, hemC and hemD are assembled in a pFN467 derived vector.

Overproduction of urógen III in recombinant E. coli cells gives rise to fluorescent cells, which can be easily visualized under UV light.

Assembly of Haem and Precorrin Biosynthetic Genes in Functional Synthesis Pathways

In this embodiment, urógen methyltransferase (cobA) from Pseudomonas denitrificans is cloned and expressed either under the control of the lac- or tac-promoter in urógen III overproducing recombinant E. coli cells, for the synthesis of precorrin-2. Further methylation of percorrin-2 to precorrin-3 is catalyzed by cobI, also from Pseudomonas denitrificans. For precorrin-3 synthesis, the cobA gene is inserted into a pACYC184 derived low-copy vector and recombinant E. coli cells, producing precorrin-2, complemented with the cobI gene.

Similarly, genes necessary for protohaem biosynthesis (hemE, hemF, HEM14 and hemH) is either cloned and assembled for expression on a pAC184 derived vector or for complementation, expressed on a pUC- or pKK-based vector, depending on which tetrapyrrole is produced and which gene is modified by directed evolution.

Directed Evolution of Protoporphyrinogen Oxidase (Aromatase)

Although genes for bacterial aromatase are known, the HEM14 gene from S. cerevisiae coding for this enzyme is used, since bacterial aromatases are heterotrimers, while yeast aromatase is a monomer.

Protoporphyrinogen oxidase catalyzes three, step-wise desaturations, each with the loss of a hydride from one ring linking C-atom and a proton from the pyrrole N of protoporphyrinogen IX followed by tautomerization to give the aromatic protoporphyrin IX. Looking at the catalyzed desaturation reactions, this enzyme can be evolved to accept coproporphyrinogen III, which differs only in its side chains from protoporphyrinogen III. Desaturation of this substrate is detectable by changes in absorption and fluorescence. Accordingly, desaturation of urógen III is possible by variants of aromatase obtained by directed evolution.

Another target for the directed evolution of aromatase is the desaturation of either precorrin-2 or precorrin-3. During the oxidation of protoporphyrinogen IX by aromatase, only the C-atoms linking rings A, B, C and D get oxidized, but not the C-atom linking rings D and A. This might be related to the inverse side-chain arrangement of ring D. In precorrin-2 and precorrin-3, though, only the C-atom linking ring D with ring A can possibly be desaturated. An enzyme variant capable to desaturate these positions would give rise to novel interesting tetrapyrroles.

Directed Evolution of Ferrochelatase

As in the case of evolved aromatase variants, novel variants of ferrochelatase can result in the production of new tetrapyrroles with interesting new light-absorbing and fluorescent properties. Ferrochelatase predominantly inserts Fe²⁺ in protoporphyrin IX, although it also inserts other metal ions like Co²⁺, Ni²⁺ and Zn²⁺ at lower rates, while Cu²⁺, Mn²⁺, Pb²⁺ and Hg²⁺are not inserted. By directed evolution of ferrochelatase different synthesis goals are addressed: ferrochelatase is evolved to insert i) Co²⁺, Ni²⁺ or Zn²⁺ at similar rates as Fe²⁺ or ii) to insert Cu²⁺ or Mn²⁺, which are not inserted by the wildtype enzyme; iii) the wildtype or any of the novel variants is adapted to other substrates exhibiting a similar reduction state as protoporphin IX, e.g novel tetrapyrroles produced by aromatase variants. Ferrochelatase is one of the few enzymes involved in tetrapyrrole synthesis which three-dimensional structure has been elucidated (Al-Karadaghi, S., et al., Structure 1997;5:1501-1510). Hence, comparison of enzyme variants and wildtype enzyme will certainly provide new information on how this enzyme functions.

Directed Evolution of Side-chain Modifying Enzymes

Further diversity of tetrapyrrole synthesis is expected by adapting the side-chain modifying enzymes urógen III decarboxylase (hemE) and coproporphyrinogen III oxidase (hemF) to new substrates.

Hence, hemE is evolved to adapt precorrin-2 or precorrin-3 as substrates and hemF to accept urógen III or precorrin-2 and precorrin-3, respectively, as substrates. Especially decarboxylation of propionate- to vinyl-residues by hemF of other tetrapyrroles than coproporphyrinogen might lead to interesting tetrapyrroles due to possible tautomerization reactions.

### Development of Tetrapyrrole Analytical and Screening Methods

Tetrapyrroles not only exhibit characteristic light absorption spectra, but also distinct fluorescent properties. Most modifications of the tetrapyrrole ring system by oxidation, metal chelation or side-chain modifications will result in a different delocalization state of the ring system and thus influence its fluorescent and light absorption properties. Therefore, light absorption and fluorescence serves as ideal tools for tetrapyrrole analysis (along with HPLC and NMR) and screening.

Especially, fluorescence of recombinant E. coli cells producing tetrapyrroles or of cell extracts can be used for sensitive detection in a screen. Hence, methods based on absorption and fluorescence are developed for the screening of large libraries. This can either be done visually as a plate screen or in a microtiter plate based assay, using either a conventional plate reader for absorption measurements or a much more sensitive fluorescence plate reader. Also digital imaging can be employed, allowing for the screening of very large libraries.

Prior to directed evolution of any biosynthetic enzyme for the synthesis of novel tetrapyrroles, the absorption and fluorescent properties of every tetrapyrrole (precorrin-2, precorrin-3, coproporphyrinogen III, protoporphyrinogen IX, protoporphyrin and protohaem IX) serving as substrates for enzymes to be modified, can be analyzed and compared to published properties. In addition, extraction methods for isolation and HPLC methods can be established based on literature methods.

### Aminoglycosides

### General

Aminoglycosides are a group of broad-spectrum antibiotics active against many aerobic gram-negative and some gram-positive bacteria. They contain an amino sugar, and an amino-or guanido-substituted inositol ring which are attached by a glycosidic linkage to a hexose nucleus, resulting in a polycationic and highly polar compound. Common examples of aminoglycosides are streptomycin, gentamicin, amikacin, kanamycin, tobramycin, netilmicin, neomycin, and framycetin.

### Aminoglcoside Biosynthetic Genes

Aminoglycosides are mostly produced by fungi or actinomycetes like bacteria belonging to the genus Streptomyces. The first discovered aminoglycoside was streptomycine from Streptomyces griseus. Its structure contains the aminocyclitol streptamine whose two amino groups are bound as guanidine substituents, making stridine. Other aminoglycoside antibiotics are based on the aminocyclito 2-deoxystreptamine (e.g. gentaminicin C1 from Micromonospora purpurea) Both, streptamine and 2-deoxystreptamine are derived from glucose-6-phosphate. Streptamine biosynthesis involves oxidation of the 5-OH and generation of an enolate anion, followed by an attack of the enolate anion on to the C-1 atom to form a cyclohexane ring. Reduction and hydrolysis of the phosphate, followed by oxidation/transamination reactions produce streptamine. Incorporation of the amidino groups from argenine produces streptidine. The biosynthesis of 2-deoxystreptidine is similar. These reactions are catalyzed by myo-Inositol-1-phosphate synthase, myo-Inositol-1 (or 4)-monophosphatase, scyllo-Inosamine kinase, minocyclitol de amidinotransferase and other yet unknown enzymes. The other components of streptomycin, namely L-streptose and 2-deoxy-2-methylamino-L-glucose are also derived from glucose-6-phosphate. For a graphical display of the biosynthesis pathways, including enzymes, genes and biochemical reactions, see World Wide Web at genome.ad jp/kegg/pathway/map/map00521.html.

Some non-limiting examples genes encoding for enzymes involved in the aminoglycoside biosynthetic pathway, which are contemplated for modification according to the invention, are listed in Table 7.

TABLE 7
Selection of Sequences Encoding Aminoglycoside Biosynthesis Enzymes

| ENZYME/PATHWAY | ORGANISM | ACCESSION NO. |
|---|---|---|
| Spectinomycin biosynthesis | Streptomyces flavopersicus | U70376 |
| ORF3, ORF2 | Streptmyces griseus | AB023785 |
| BimS, B1mT, BlinD | Streptomyces bluensis | F126354 |
| 5′hydroxystreptomycin biosynthesis | Streptomyces glaucescens | AJ006985 |
| strA, trB1, strD, strF, strG, strH, strI, | Streptomyces griseus | Y00459 |
| strK, strR, strS strT | | |
| fosfomycin biosynthesis | Streptomyces wedmorensis | AB016934 |
| fortimycin KL1 methyltransferase | Micromonospora | D49442 |
| | ohvasterospora | |
| formimidoyl fortimycin A synthetase | M. olivasterospora | D10050 |

Literature describing the structure and/or function of these genes can be found in Distler J, et al., Mol Gen Genet June 1987;208(1-2):204-10; Ohnuki T, et al., J Bacteriol October 1985; 164(1):85-94; Shiro M, et al., Biochim Biophys Acta Feb. 7, 1996;1305(1-2):44-8; Beyer S. et al., Mol Gen Genet Apr. 10, 1996;250(6):775-84; Beyer S, et al., Eur J Biochem Dec. 15, 1998; 258(3):1059-67; Mansouri K, et al., Mol Gen Genet September 1991;228(3):459-69; Peschke Mol Microbiol Jun. 16, 1995;(6):1137-56; Distler J, et al., Nucleic Acids Res Oct. 12, 1987; 15(19):8041-56; Ahlert J, et al., Arch Microbiol August 1997;168(2):102-13; Kuzuyama T, et al., J Antibiot (Tokyo) October 1995;48(10):1191-3; Kuzuyama T, et al., J Antibiot (Tokyo) September 1993; 46(9):1478-80; Hidaka T, et al., Mol Gen Genet Nov. 27, 1995;249(3):274-80; Dairi T, et al., Mol Gen Genet December 1992;236(1):49-59; Ohta T, et al., J Antibiot (Tokyo) July 1992;45(7):1167-75.

### Directed Evolution of Aminoglcoside Biosynthetic Pathways

Analogous to the molecular pathway breeding described above, novel biosynthetic capacities of aminoglycosides can be explored by assembly of biosynthetic genes from different microbial sources and in vitro mutagenesis of selected enzymes. Preferably, biosynthetic genes from reasonably well understood enzyme functions are used in the initial experiments.

### Non-ribosomal peptide synthesis

### General

Many naturally occurring peptides are not produced via ribosomal biosynthesis, but by a more individualistic sequence of enzyme-controlled processes. Useful properties of many such non-ribosomally produced peptides include antibiotic activities. For instance, vanomycin is a glycopeptide antibiotic produced by Streptomyces orientalis, which has activity against gram-positive bacteria, especially resistant strains of staphylococci and streptococci. Polymyxins are a group of cyclic polypeptides produced by Bacillus species, which have been used for treatment of infections with gram-negative bacteria, as well as in various preparations for topical use. Actinomycin D is an antibiotic produced by the funguslike bacterium Streptomyces parvallum, which inhibits RNA transcription in eukaryotes and has antitumour properties, so it is of ten used in conjunction with other drugs in chemotherapy. Other microbially produced polypeptide mixtures used clinically include Bacitracin, Tyrothricin, and Capreomycin.

### Biosynthetic Genes for Non-Ribosomal Peptide Synthesis

Non-ribosomal peptide synthesis is apparently carried out by multi-enzyme complexes. Each amino acid, added depending on enzyme specificity, is activated by conversion to an AMP-ester. This derivative is subsequently bound to the enzyme through thioester linkages, oriented so that a sequential series of peptide bonds are formed before the peptide is released from the multi-enzyme complex.

Some non-limiting examples of genes encoding for enzymes involved in non-ribosomal peptide synthesis pathways, which are contemplated for modification according to the invention, are listed in Table 8.

TABLE 8
Selection of Sequences Encoding Non-Ribosomal Protein Biosynthesis Enzymes

| ENZYME/PATHWAY | ORGANISM | ACCESSION NO. |
|---|---|---|
| dehydrogenase, ligase, carboxylase, | Bacillus subtilis | AF218939 |
| hydroxymethylglutaryl-CoA lyase, | | |
| hydroxybutyryl-dehydratase | | |
| ppsE, yngL, yngK, yotB, yngI, yngH, | Bacillus subtilis | Y13917 |
| yngG, yngF, ppsD, yngE | | |
| gramicidin S synthetase | B.subtilis | Z34883 |
| gramicidin S Biosynthesis | B.brevis | M29703 |
| actinomycin synthetase III | Streptomyces chrysomallus | AF204401 |
| actinomycin synthetase I | Streptomyces chrysomallus | AF134587 |

Literature describing the structure and/or function of these genes can be found in Quadri L E, et al., Biochemistry Nov. 9, 1999;38(45):14941-54; Reimmann C, et al., Microbiology November 1998;144 (Pt 11):3135-48; Suo Z, et al., Biochemistry Oct. 19, 1999;38(42):14023-35; Serino L, et al., J Bacteriol January 1997;179(1):248-57; Shaw-Reid C A, et al., Chem Biol Jun. 6, 1999; (6):385-400; Fernandez-Moreno M A, et al., J Bacteriol November 1997;179(22):6929-36; Schwartz D, et al., Appl Environ Microbiol February 1996;62(2):570-7; Wohlleben W, et al., Gene Jun. 15, 1992; 115(1-2):127-32; Grammel N, et al., Biochemistry Feb. 10, 1998;37(6):1596-603; Strauch E, et al., Gene 1988;63(1):65-74; Behrmann I, et al., J Bacteriol 1990 September; 172(9):5326-34; Pospiech A, et al., Microbiology August 1995;141 (Pt 8):1793-803; Bernhard F, et al., DNA Seq 1996;6(6):319-30; Schauwecker F, et al., J Bacteriol May 1998;180(9):2468-74; Pospiech A, et al., Microbiology April 1996; 142 (Pt 4):741-6; Chong P P, et al., Microbiology January 1998;144 (Pt 1):193-9; de Crecy-Lagard V, et al., Antimicrob Agents Chemother September 1997;41(9):1904-9; Haese A, et al., Mol Microbiol Mar. 7, 1993;(6):905-14; Perkins J B, et al., J Bacteriol June 1990;172(6):3108-16; Butlrt M J, et al., Appl Environ Microbiol August 1995;61(8):3145-50; Weber G, et al., Curr Genet Aug. 26, 1994; (2):120-5; Gutierrez S, et al., J Bacteriol April 1991;173(7):2354-65; Pfennig F, et al., J Biol Chem Apr. 30, 1999;274(18):12508-16; Kovacevic S, et al., J Bacteriol February 1989;171(2):754-60; Saito F, et al., J Biochem (Tokyo) August 1994;116(2):357-67; Yu H, et al., Microbiology December 1994; 140 (Pt 12):3367-77; Stachelhaus T, et al., J Biol Chem Mar. 17, 1995;270(11):6163-9; de Ferra F, et al., J Biol Chem Oct. 3, 1997;272(40):25304-9; Stein T, et al., J Biol Chem Jun. 28, 1996; 271(26):15428-35; NakataK, et al., FEMS Microbiol Lett Jan. 1, 1989;48(1):51-5; Wessels P, et al., Eur J Biochem Dec. 15, 1996;242(3):665-73; Pelzer S, et al., J Biotechnol Aug. 11, 1997; 56(2):115-28; Blanc V, et al., Mol Microbiol Jan. 23, 1997;(2):191-202; Parquet C, et al., Nucleic Acids Res Jul. 11, 1989;17(13):5379; Neilan B A, et al., J Bacteriol July 1999;181(13):4089-97; Lacalle R A, et al., EMBO J Feb. 11, 1992;(2):785-92; Mao Y, et al., J Bacteriol April 1999;181(7):2199-208; Konig A, et al., Eur J Biochem Jul. 15, 1997;247(2):526-34; Billman-Jacobe H, et al., Mol Microbiol September 1999;33(6):1244-53; Du L, et al., Chem Biol Aug. 6, 1999;(8):507-17; Nishizawa T, et al., J Biochem (Tokyo) September 1999;126(3):520-9; Tosato V, et al., Microbiology November 1997;143 (Pt 11):3443-50; Tognoni A, et al., Microbiology Mar. 1995;141 (Pt 3):645-8; Yoshida K, et al., DNA Res Dec. 31, 1995;2(6):295-301; Steller S, et al., Chem Biol Jan. 6, 1999;(1):31-41; Lin GH, et al., J Bacteriol March 1998;180(5):1338-41; Cosmina P, et al., Mol Microbiol Mar. 8, 1993;(5):821-31; Schneider A, et al., Arch Microbiol May 1998;169(5):404-10; Steller S, et al., J Chromatogr B Biomed Sci App Jan. 14, 2000;737(1-2):267-75; Kratzschmar J, et al., J Bacteriol October 1989; 171(10):5422-9; Krause M, et al., J Bacteriol October 1988;170(10):4669-74; Saito F, et al., J Biochem (Tokyo) August 1994;116(2):357-67.

### Directed evolution of Biosynthetic Pathways for Non-Ribosomal Peptide Synthesis

Analogous to the molecular pathway breeding described above, novel capacities of enzymes involved in non-ribosomal protein synthesis can be explored by assembly of biosynthetic genes from different microbial sources and in vitro mutagenesis of selected enzymes. Preferably, biosynthetic genes from reasonably well understood enzyme functions are used in the initial experiments.

### Biodegradation pathways for aromatic compounds

### General

Over the past 15 years many catabolic enzymes and pathways for the degradation of aromatic xenobiotics have been described on a molecular level. Comparison of the major pathways involved in biodegradation of aromatic compounds reveals that different enzymes carry out the initial conversion steps but that the reaction products are farther metabolized by a limited number of central routes yielding intermediates such as protocatechuates or (substituted) catechols. The activation of the aromatic nucleus through the introduction of two hydroxyl groups is a general requirement for the initiation of aerobic degradation. These dihydroxylated compounds are then channeled either in a meta- (extra-diol cleavage) or ortho- (intra-diol cleavage) cleavage pathway, which ideally leads to intermediates of central metabolic routes, such as the tricarboxylic acid cycle (TCA) (See FIG. 11). For a review see Ellis, L. B. M., Hershberger, C. D. and Wackett, L. P. (1999) Nucl. Ac. Res. 27:373-376; Van der Meer, J. R., de Vos, W., Harayama, S. and Zehnder, A. J. B. (1992) Microbiol. Rev. 56:677-694; Lal, R., Lal, S., Dhanaraj, P. S. and Saxena, D. M. (1995) Adv. Appl. Microbiol. 41:55-95.

Microbial degradation of aromatic xenobiotics is chromosomal- as well as plasmid-mediated. For instance, genes of benzoate catabolism are chromosomally encoded while those for toluene or xylene degradation are plasmid encoded. The chromosomal genes in general mediate the degradation through the ortho-pathway, whereas the plasmid encoded enzymes degrade these compounds through the meta-pathways (Lal, R., et al., Adv. Appl. Microbiol. 1995;41:55-95). The first biodegradation enzymes were identified as plasmid-encoded genes in Pseudomonas species. In the meantime a variety of plasmids, encoding catabolic genes, have been isolated from various Pseudomonas species. The plasmid pWWO from Ps. putida PaW1 is the most extensively studied plasmid and was first described in 1974 (Williams, P. A. and Murray, K., J. Bacteriol. 1994;120:416-423). Because of its role in toluene degradation this plasmid was designated as TOL plasmid, although other compounds such as xylenes are also degraded.

The organization of catabolic genes in operons and their frequent location on transposons contributed to the interchange of genetic material between chromosome and plasmid as well between different microorganisms. In fact, mixing of pathway modules of different microorganisms is thought to be the main driving force behind the adaptation of microorganisms to novel xenobiotics (Van der Meer, J. R., et al., Microbiol. Rev. 1992;56:677-694, Van der Meer, J. R. (1997) Antonie van Leeuwenhook 71:159-178).

The improved and tailor-made metabolic routes provided by the invention of fers exciting possibilities to overcome many of the problems associated with the handling of harmful waste products. To improve, for instance, the rate of pollutant removal it is necessary to determine the rate-limiting enzymatic or regulatory step in a multi-step pathway and increase expression and/or catalytic performance of this enzyme (or enzymes). Mono- and dioxygenases have been identified as the rate-limiting steps in the degradation of aromatic compounds (Timmis, K. N., Steffan, R. J. and Untermann, R. (1994) 48:525-557; Sheridan, R., Jackson, G. A., Regan, L., Ward, J. and Dunnill, P. (1998) Biotechnol. Bioeng. 58:240-249). Improvement of catalyst performance (e.g. activity, stability, and substrate specificity), though, will require engineering the individual catalysts. Expansion of pathways to new substrates is achieved by altering of substrate spectra of participating enzymes in a pathway and assembly of enzymes to a create novel pathways according to the invention.

### Bioderadation Pathways

In this embodiment of the invention, catabolic enzymes are combined in new pathways and their catalytic performance evolved according to, e.g., a specific environmental pollutant to be degraded. Instead of combining pathway modules, enzymes from different microorganism is assembled and evolved in order to design efficient, novel biodegradation routes. Especially the upper-pathway elements, which funnel degradation intermediates into the meta- or ortho-cleavage routes, are simplified. To this end, single- or two-component monooxygenases, hydroxylases or dehalogenases replace the multi-component mono- or dioxygenases. A simplified pathway offers the advantage to be more easily genetically handled and evolved by in vitro techniques towards new substrates and better performance.

Standard E. coli expression systems may be used during molecular evolution and pathway assembly, the finally designed pathways is preferably placed under the control of a regulatory circuit efficiently induced by the aromatic compound to be degraded. Therefore, appropriate regulated promoters are preferably developed. In a final step, these pathways are implemented in microorganisms, preferably Pseudomonas, suitable for bioremedation processes. Stable chromosomal integration can be achieved by minimized transposons, which contain selection markers other than antibiotic resistance genes and lacking the resolvase gene (De Lorenzo, V., Herrero, M., Sanchez, J. M. and Timmis, K. N. (1998) FEMS Microbiol. Ecology 27:211-224).

### Directed Evolution of Bioderadation Pathways

The first objective in directed evolution of novel catabolic pathways is preferably, although not necessarily, the cloning and expression of the necessary catabolic genes in E. coli. Hence, genes are either be isolated by PCR from the respective microorganism or, if this microorganism is not available from a strain collection, requested from researchers working with the genes.

For the expression of all these different catabolic genes in E. coli, two modified expression vectors based on pUC (lac-promoter, pUCmod) and pKK (tac-promoter, pKKmod) with optimized cloning sites, shine-dalgamo sequence and different promoter strengths were designed. In addition, a second low-copy number plasmid (pACmod) based on pACYC184 and compatible to the pUC and pKK-based vectors was designed for complementation in E. coli (Schmidt-Dannert, C. and Arnold F. H., in preparation). A third low-copy number plasmid vector pFN467, compatible to the previous ones, is modified (pFNmod) to allow the assembly and handling of catabolic routes involving more than six genes. While pACmod or pFNmod serve as vectors for the expression of assembled catabolic genes (each gene under the control of its own promoter), pUCmod or pKKmod are used for library creation following in vitro mutagenesis or gene shuffling of the target genes.

Crucial for all further steps of tailoring enzyme functions for biodegradation is knowledge of substrate specificities and activities of those enzymes selected for molecular pathway breeding. Since information on substrate spectrum and activity apart from one main catalytic function is available only for a few well-characterized catabolic enzymes, substrate spectrum and activity of single enzymes and assembled enzymes is investigated. Hence, HPLC-based analytical methods are developed according to methods described in literature (Van der Meer, J. R., de Vos, W., Harayama, S. and Zehnder, A. J. B. (1992) Microbiol. Rev. 56:677-694; Sheridan, R., Jackson, G. A., Regan, L., Ward, J. and Dunnill, P. (1998) Biotechnol. Bioeng. 58:240-249). In addition, efficient screening methods for molecular evolution of enzymes need to be developed based either on a direct screening of E. coli clones expressing desired enzyme on agar plates or, more likely, on a spectrophotometrical screen in a microtiter plate format. Numerous spectrophotometrical assays for the detection of phenolic compounds, catechols, halogenes, muconic acid etc. have been described (Mars, A. E., Kingma, J., Kaschabek, S. R., Reineke, W. and Janssen D. B. (1999) J. Bacteriol. 181:1309-1318; Davis, J., Vaughan, D. H. and Cardosi, M. F. (1995) Anal. Proc. 32:423-426; Parke, D. (1992) Appl. Environm. Microbiol. 58:2694-2697; Bertoni, G., Bolognese, F., Galli, E. and Barbieri, P. (1996) 62:3704-3711; Khalaf, K. D., Ba, H., Moralesrubio, A. and Delaguardia, M. (1994) TALANTA 41:547-556; Cheregi, M. and Danet, A. F. (1997) Anal. Lett. 30:2847-2858) and can be adapted. In addition, it may be possible to use a dedicated cell-sorter, by exploiting the internal fluorescence of some of the intermediates.

The invention provides for the following non-limiting embodiments, of which two deals with the design of new pathways and the third with the development of inducible promoters for biodegradation.

Molecular Pathway Breeding for the Degradation of Non-Halogenated Aromatic Compounds

As outlined above, non-halogenated aromatic compounds such as toluene, phenol, xylenes are degraded by the activation of the aromatic nucleus through the introduction of two hydroxyl groups and thus producing (substituted) catechol. Two enzymes, a multi-component dioxygenase or hydroxylase (e.g. four-component toluene-dioxygenase or phenol-hydroxylase) and a dihydrodiol dehydrogenase, usually catalyze catechol synthesis. However, in other microorganisms a single-component phenol monooxygenase is capable to hydroxylate phenol to catechol (Nurk, A., Kasak, L. and Kivisaar, M. (1991) Gene 102:13-18; Ohlsen, R. H., Kukor, J. J. and Kaphammer, B. (1994) J. Bacteriol. 176:3749-3756). Furthermore, monooxygenases have been described to introduce not only one but two hydroxyl to an aromatic nucleus and to remove chlorine groups by hydroxylation (S. Fetzner (1998) Appl. Microbiol. Biotechnol. 50:633-657). The phenol hydroxylase from e.g. Ps. pickettii hydroxylates methylated and chlorinated phenol derivatives (Ohlsen, R. H., Kukor, J. J. and Kaphammer, B. (1994) J. Bacteriol. 176:3749-3756). Based on these facts it should not only be possible to develop a pathway for efficient degradation of phenol-derivatives by directed evolution of phenol-monooxygenase and funneling of the catechols into the meta-pathway, but also to adapt phenol-monooxygenase to substrates such as xylene, toluene, cresol and benzene.

Cloning of catabolic genes and construction of expression vectors. Genes for phenol monooxygenase and meta-cleavage pathway are isolated by PCR from the respective microorganisms based on published nucleotide sequences or requested from researchers working with these genes. All genes are cloned in either pUCmod or pKKmod and assembled to pathways by inserting in low-copy vectors pACmod and pFNmod, as described above.

The following genes are cloned:

Phenol-monooxygenase from Pseudomonas sp. EST1001 and Pseudomonas pickettii PKO1.

Catechol 2,3 dioxygenase from Bacillus stearothermophilus and Pseudomonas putida UCC-2.

2-Hydroxymuconic semialdehyde dehydrogenase, 2-oxo-pent-4-enolate hydroxylase and 4-hydroxy-2-oxovalerate-aldolase from Pseudomonas putida and Acinetobacter sp.

Functional expression of phenol-monoxygenases and investigation of enzyme activity and substrate specificity. Both phenol-monooxygenases from Ps. sp. and Ps. picketti are expressed in E. coli and their hydroxylating activity with phenol, xylene, cresol, toluene and benzene as substrates is investigated (see objective 3). The enzyme with the broadest substrate specificity, and hence the largest potential to be evolved for efficient hydroxylation of substrates other than phenol, is chosen for further work.

Development of analytical and screening methods. HPLC-methods are developed based on published methods (Van der Meer, J. R., de Vos, W., Harayama, S. and Zehnder, A. J. B. (1992) Microbiol. Rev. 56:677-694; Sheridan, R., Jackson, G. A., Regan, L., Ward, J. and Dunnill, P. (1998) Biotechnol. Bioeng. 58:240-249) for the accurate analysis of enzyme activities, e.g. hydroxylation of aromatic compounds and cleavage of substituted and unsubstituted catechols by wildtype or variants of phenol-monooxygenase and catechol 2,3 dioxygenase, respectively. Furthermore, HPLC-analysis, and if necessary NMR- and MS-analysis, is advantageous to investigate substrate flow through the meta-pathway and to detect the enzymatic steps which control intermediate conversion and hence need to be adapted by molecular evolution.

For the directed evolution of individual enzymes, efficient methods for screening large numbers of E. coli clones expressing enzyme variants should be established. Hence, microtiter plate based spectrophotometrical screens can be developed. Spectrophotometrical detection methods for phenols, catechols and diverse other intermediates have been described (Mars, A. E., Kingma, J., Kaschabek, S. R., Reineke, W. and Janssen D. B. (1999) J. Bacteriol. 181:1309-1318; Davis, J., Vaughan, D. H. and Cardosi, M. F. (1995) Anal. Proc. 32:423-426; Parke, D. (1992) Appl. Environm. Microbiol. 58:2694-2697; Bertoni, G., Bolognese, F., Galli, E. and Barbieri, P. (1996) 62:3704-3711; Khalaf, K. D., Ba, H., Moralesrubio, A. and Delaguardia, M. (1994) TALANTA 41:547-556; Cheregi, M. and Danet, A. F. (1997) Anal. Lett. 30:2847-2858). These methods can be adapted to a microtiter plate format. Positive enzyme variants identified in these spectrophotometrical screens can be more accurately analysed by HPLC methods.

Assembly of meta-pathway and investigation of substrate acceptance and complementation with phenol-monooxygenase. Catechols produced by the action of phenol-monooxygenase are funneled into the meta-pathway. In contrast to the ortho-cleavage route, methyl-catechols derived from xylene, toluene or cresol can be completely degraded through the meta-pathway and no dead-end products are formed (Timmis, K. N., Steffan, R. J. and Untermann, R. (1994) 48:525-557).

In order to investigate substrate specificities and enzyme activities of the catechol 2,3 dioxygenases, they are expressed separately and enzyme properties investigated by HPLC-analysis. The enzyme with the broadest substrate spectrum can be chosen for assembly into the meta-cleavage pathway.

The meta-cleavage enzymes 2-hydroxymuconic semialdehyde dehydrogenase, 2-oxo-pent-4-enolate hydroxylase and 4-hydroxy-2-oxovalerate-aldolase, either from Ps. putida or Acinetobacter sp., can be assembled on pACmod. Substrate flow through the meta-pathways can be analysed by HPLC and accumulation of intermediates investigated. Those enzymes, which show the broadest substrate spectrum, can be selected for the final assembly of a pathway

Finally, the assembled meta-cleavage pathway on pACmod can be complemented with pUCmod or pKKmod expressing the gene for phenol-monooxygenase and degradation of phenol through this pathway can be investigated.

Improvement of phenol degradation by directed evolution of phenol-monooxygenase and catechol 2,3 dioxygenase. The initial hydroxylation of the aromatic nucleus and ring-cleavage have been reported to be the rate limiting steps during degradation of aromatic compounds (Timmis, K. N., Steffan, R. J. and Untermann, R. (1994) 48:525-557). Thus, improvement of the catalytic activity of phenol-monooxygenase and catechol 2,3 dioxygenase can result in an increased biodegradation rate of phenol. To this end, both enzymes are evolved by random mutagenesis for variants with increased catalytic activity. Screening of the created E. coli library expressing either phenol-monooxygenase variants or catechol 2,3 dioxygenase variants is done spectrophotometrically (phenol, catechol) or fluorimetrically (phenol) in a microtiter plate format. Variants with improved catalytic activity are then introduced into the complete degradation pathway and biodegradation rates compared to the pathway containing the wildtype enzymes.

Altering the substrate specificity of phenol-monooxygenase by directed evolution. Although phenol is the preferred substrate for phenol-monooxygenases, these enzymes also hydroxylate other substrates such as chlorophenols and cresols. Thus, directed evolution of phenol-monooxygenase can lead to variants with an altered substrate spectrum. E. coli libraries expressing phenol-monooxygenase variants are screened for variants with enhanced hydroxylating activity for different methylated phenols, benzenes, toluenes and xylenes. A library can be screened simultaneously with different substrates, possibly allowing the identification of variants with high hydroxylating activity toward several substrates. Evolved variants with altered hydroxylating activities are introduced into the meta-pathway, and degradation of substrates other than phenol can be investigated by HPLC.

Additionally, phenol-monooxygenase can be evolved to hydroxylate chloroaromatic compounds. The resulting catechols possibly need to be funneled into the ortho-pathway (see project II) due their inhibitory effect on catechol-2,3-monooxygenases. However, recently a catechol 2,3 dioxygenase from Ps. putida GJ31 has been described to convert 3-chlorocatechol (Mars, A.E., et al., J. Bacteriol. 1999;181:1309-1318). Thus, a catechol 2,3 dioxygenase is evolved which is capable to convert chlorocatechols.

Adaptation of meta-pathway enzymes to new substrates. It is most likely that certain enzymes of the meta-pathway will have to be adapted to substituted catechols (methyl-catechol can be degraded). Hence, depending on the results from the HPLC-analysis, individual enzymes of this pathway is evolved to allow efficient degradation of new substrates.

Molecular Pathway Breeding for (Poly)Chlorophenol Degradation

Chlorophenols, including pentachlorophenol (PCP), represent a major group of environmental pollutants that are not easily degraded by microorganism. However, several microorganisms have been isolated to degrade PCP, and the metabolism of chlorophenol degradation has been studied. Two major classes of metabolic pathways for chlorophenol degradation have been identified. Mono- and dichlorophenols are usually degraded analogous to non-halogenated aromatic compounds by ring-activation through multi-component dioxygenases and funneling into the ortho-pathway. On the other hand, most polychlorinated phenols are degraded through a chlorohydroxyquinol intermediate before ortho-cleavage of the aromatic ring. Degradation of PCP to chlorohydroxyquinol has been very recently elucidated for the first time on both an enzymatic and a molecular level for Flavobacterium sp.ATCC3972, summarized in (S. Fetzner (1998) Appl. Microbiol. Biotechnol. 50:633-657). Three enzymes are involved in PCP degradation. The first enzyme, PCP 4-monooxygenase, converts PCP to tetrachloro-p-hydroquinone. Next, tetrachloro-p-hydroquinone reductive dehalogenase converts tetrachloro-p-hydroquinone to 2,6-dichloro-p-hydroquinone. In a final reaction, 2,6-dichloro-p-hydroquinone is converted to 6-chlorohydoxyquinol (6-CHQ) by a 2,6-dichlorohydroquinone chlorohydrolase. 6-CHQ is thought to undergo ortho-ring cleavage in Flavobacterium sp. for further degradation.

Accordingly, an efficient pathway for the degradation of polychlorinated phenols can be assembled and evolved. The three genes necessary for PCP dehalogenation, may, for instance, be assembled in a functional pathway in E. coli. Thus, functional PCP degradation to 6-CHQ can be established in E. coli. In order to allow further degradation of 6-CHQ through ortho-ring cleavage, genes necessary for ortho-ring cleavage of chlorocatechols are assembled in a functional pathway. As outlined in FIG. 11, chlorocatechols are degraded through a modified ortho-pathway.

Enzymes known to degrade chlorocatechols are used for ortho-ring cleavage as previously reviewed (Reineke, W. (1 998) Ann. Rev. Microbiol. 52:287-331; Schlohmann, M. (1 994) Biodegradation 5:301-321). In particular, chlorocatechol 1,2 dioxygenase, chlroromuconate cycloisomerase, dienelactone hydrolase and maleylacetate reductase degrade di- and trichlorocatechols to form 3-oxoadipate or chlorine substituted 3-oxooadipate. This pathway includes two dechlorination steps: dechlorination at position 4 or 5 by the action of the chloromuconate cycloisomerase and at position 2 by the action of the maleylacetate reductase. The final degradation product 3-oxoadipate needs to be further metabolized by the 3-oxoadipate (B-ketoadipate) pathway consisting of the 3-oxoadipate:succinyl-CoA transferase and 3-oxoadipyl-CoA thiolase in order to reach the tricarboxylic acid cycle.

Following an investigation of biodegradation of PCP and other polychlorinated aromatics through both upper- and ring-cleavage pathway by HPLC-analytic, rate-limiting enzymes can be optimized by directed evolution and substrate specificities of enzymes evolved for the efficient degradation of various polychlorinated phenols.

Cloning of catabolic genes and construction of expression vectors. Genes for PCP degradation and ortho-ring cleavage can be isolated by PCR from the respective microorganisms based on published nucleotide sequences. All genes can be cloned in either pUCmod or pKKmod and assembled to pathways by inserting in low-copy vectors pACmod and pFNmod, as described above.

The following genes for PCP degradation can be cloned:

PCP 4-monooxygenase (pcpB), tetrachloro-p-hydroquinone reductive dehalogenase (pcpC)

2,6-dichlorohydroquinone chlorohydrolase (pcpA) from Flavobacterium sp. ATCC 39723

The following genes for ortho-ring cleavage can be cloned:

chlorocatechol 1,2 dioxygenase from Ralstonia eutropha and Ps. putida

chloromuconate cycloisomerase from Ralstonia eutropha and Ps. putida

dienelactone hydrolase Ralstonia eutropha and Ps. sp.

maleylacetate reductase from Ralstonia eutropha and Ps. sp. B13.

The expression in E. coli of the cloned catabolic genes under the control of either the lac- or tac-promoter is checked.

Assembly of a functional PCP-degradation pathway and investigation of PCP degradation. Following the verification of expression of the PCP-degradation genes from Flavobacterium sp. in E. coli, these genes are assembled in pACmod to create a pathway. Each gene is expressed under the control of either the lac- or the stronger tac-promoter. Degradation of PCP and other polychlorinated mono-aromatic compounds by this pathway are investigated by HPLC-analysis as well as MS- or NMR-analysis (see below), thereby investigating the substrate specificities of the individual enzymes and the rate limiting steps of dechlorination. A careful determination of dechlorination of PCP and other polychlorinated phenols is necessary to identify those enzyme in the pathway, which need to be evolved in terms of increased activity or substrate specificity.

Development of analytical and screening methods. HPLC-methods are developed based on published methods (Van der Meer, J. R., de Vos, W., Harayama, S. and Zehnder, A. J. B. (1992) Microbiol. Rev. 56:677-694; Sheridan, R., Jackson, G. A., Regan, L., Ward, J. and Dunnill, P. (1998) Biotechnol. Bioeng. 58:240-249) for the accurate analysis of enzyme activities of both the dechlorination of PCP and other polychlorinated compounds and the further degradation of chlorocatechols by ortho-ring cleavage. HPLC-analysis as well as MS- or NMR-analysis is needed to investigate substrate flow through both the upper dechlorination pathway and the ortho-ring cleavage pathway and the detection of enzymatic steps allowing only slow intermediate conversion or no conversion at all and hence, needing to be adapted by molecular evolution.

For the directed evolution of individual enzymes efficient screening methods, allowing the screening of large numbers of E. coli clones expressing enzyme variants, should be established. Hence, microtiter plate based spectrophotometrical screens is developed. Spectrophotometrical detection methods for phenolic compounds, chlorocatechols, chloromuconic acid and chlorid have been described in literature (Mars, A. E., Kingma, J., Kaschabek, S. R., Reineke, W. and Janssen D. B. (1999) J. Bacteriol. 181:1309-1318; Davis, J., Vaughan, D. H. and Cardosi, M. F. (1995) Anal. Proc. 32:423-426; Parke, D. (1992) Appl. Environm. Microbiol. 58:2694-2697; Bertoni, G., Bolognese, F., Galli, E. and Barbieri, P. (1996) 62:3704-3711; Khalaf, K. D., Ba, H., Moralesrubio, A. and Delaguardia, M. (1994) TALANTA 41:547-556; Cheregi, M. and Danet, A. F. (1997) Anal. Lett. 30:2847-2858). These methods can be adapted to a microtiter plate format. Positive enzyme variants identified in these spectrophotometrical screens are more accurately analysed by HPLC methods.

Assembly of ortho-pathway and investigation of chlorocatechol degradation. The genes necessary for ortho-ring cleavage of chlorocatechols are assembled on pFNmod to a functional pathway. Each gene is expressed under the control of either the lac- or the tac-promoter. Genes cloned from Ralstonia are assembled to one pathway and those cloned from Pseudomonas gives rise to a second pathway. Although information on substrate specificity of many of these enzymes is available as reviewed in (Reineke, W. (1998) Ann. Rev. Microbiol. 52:287-331), substrate specificities of the assembled pathways in E. coli should be investigated as well as substrate flux through the pathway and the accumulation of intermediates. Especially the degradation of 6-CHQ as the product of PCP-degradation through the upper-pathway needs to be investigated. Depending on the results, a final pathway is assembled with genes from both Ralstonia and Pesudomonas to yield a pathway which allows the degradation of many substituted catechols in E. coli.

Co-expression of PCP-degradation and ortho-pathway in E. coli. In order to check if both pathways, PCP-degradation (upper-pathway) and ortho-ring cleavage of chlorocatechols, function together in E. coli and lead to the degradation of PCP to 3-oxoadipate, both plasmids pACmod and pFNmod (expressing the upper-pathway and ortho-pathway, respectively) are co-transformed in E. coli and degradation of PCP and other polychlorinated aromatic compounds investigated.

Directed evolution of enzymes involved in the PCP-degradation pathway. Depending on the investigations on substrate specificities of the individual enzymes and the identification of rate-limiting enzymes as described in objective 3, two main goals are addressed by directed evolution: i) increase of enzyme activity of rate limiting enzymes towards PCP and other polychlorinated phenols and ii) adaptation of enzymes to other substrates than PCP.

While the first enzyme involved in PCP-degradation, PCP 4-monooxygenase, has a rather broad substrate specificity (Xun, L. and Orser, C. S. (1991) J. Bacteriol. 173:4447-4453; Xun, L., Topp, E. and Orser, C. S. (1992) J. Bacteriol. 174:2898-2902) is that of the 2,6-dichlorohydroquinone chlorohydrolase restricted to 2,6 dichlorohydroquinone (Lee, J.-Y. and Xun, L. (1997) J. Bacteriol. 179:1521-1524). Thus, apart from enhancing the enzyme activities necessary for effective PCP degradation, 2,6-dichlorohydroquinone chlorohydrolase are evolved to accept phenols containing chlorines at different positions.

Directed evolution of enzymes involved in ortho-ring cleavage of chlorocatechols. Similar to the molecular evolution of the enzymes involved in the upper-pathway (PCP-degradation), also the enzyme activity of rate-limiting enzymes of chlorocatechol degradation is increased by directed evolution. As outlined in project I, it is likely that the chlorocatechol 1,2 dioxygenase is the rate-limiting enzyme and hence, is evolved. In addition, individual enzymes of the ring-cleavage pathway can be adapted to efficiently degrade the chlorinated catechols produced by the upper-pathway.

Molecular Evolution of Regulatory Circuits

The designed pathways are placed under the control of a regulatory circuit efficiently induced by the aromatic compound to be degraded. Regulatory circuits have been described for several catabolic operons involved in biodegradation (Collier, L. S., Gaines, G. L. and Neidle, E. (1998) J. Bacteriol. 180:2493-2501). However, the transcriptional control of the TOL plasmid catabolic operons has so far been investigated in most detail, as reviewed by Ramos et al. (Ramos, J. L., Marques, S. and Timmis, K. (1997) Annu. Rev. Rev. Microbiol. 51:341-73). Positive regulation of the operons involved in toluene degradation is mediated by two regulator proteins XylS and XylR which belong to the XylS/AraC and NtrC families, respectively, of transcriptional regulators. Expression of the upper pathway operon for toluene degradation is controlled by XylR (cascade loop), while XylS regulates the expression of the meta-pathway operon (meta loop). Since the cascade loop, which is controlled by XylR, is much more complex than the meta loop, requires a s54-containing RNA polymerase and a DNA-bending protein integration host factor (IHF) and is subject to catabolite repression, XylS mediated regulation can be chosen for the directed evolution of regulatory circuits.

XylS is expressed in an inactive form at low constitutive levels from the Ps2 promoter. Alkylbenzoates as the primary substrates of the meta-pathway enzymes bind to XylS and activate the effector, which then binds to the Pm promoter and allows transcription of the meta-pathway operon. Both the XylS regulator and the Pm promoter have been studied in detail (Ramos, J. L., Marques, S. and Timmis, K. (1997) Annu. Rev. Rev. Microbiol. 51:341-73). XylS is composed of two domains: a C-terminal region involved in DNA-binding and a more N-terminal located recognition pocket for XylS effectors. Substituted benzoates are XylS effectors, but the positions and the type of the substituents define the binding to XylS. How the binding of the effector mediates activation of XylS and thus, binding to the Pm promoter is not yet understood.

According to the invention, the XylS regulatory circuit is evolved for the selective induction of designed catabolic pathways as developed in project I and II. Therefore, the XylS regulator gene preceded by the Ps2 promoter is cloned on a pUC-based plasmid. A reporter gene, such as the green fluorescent protein (GFP), is placed under the control of the Pm promoter on a second plasmid pACmod. Directed evolution of XylS by random mutagenesis can result in novel variants that bind effectors like phenol, toluene, xylene, benzene or chlorinated phenols and mediate transcription from the Pm promoter at low effector concentrations. Especially, induction of catabolic gene expression at low concentrations of the compound to be degraded is important for an efficient biodegradation process. The threshold concentrations for the induction of degradative pathways in microorganisms are usually higher than desirable for an efficient biodegradation process.

Cloning of XylS expression unit and construction of the reporter system. The gene encoding XylS and its promoter Ps2 can be isolated by PCR based on published nucleotide sequences and using the TOL plasmid pWWO from Ps. putida as template. This expression unit can then be cloned in a pUC-based vector devoid of the lac-promoter.

In order to check the function of XylS and screen a library of XylS variants for desired properties, a second vector expressing a reporter gene under the control of the XylS dependent Pm promoter is constructed. Therefore, the enhanced green fluorescent protein (egfp from Clonetech) can serve as a reporter protein and be inserted into the low-copy vector pACmod. The Pm promoter is fused upstream to the egfp gene. Following transformation of E. coli with pACmod, containing the egfp gene under the control of the Pm promoter, and with the second vector expressing XylS at low constitutive levels, recombinant cells can appear green fluorescent in the presence of a XylS effector.

Development of screening methods and determination of XylS effectors. A microtiter plate based screen for the identification of novel XylS variants is developed. E. coli clones expressing XylS variants capable of binding a desired effector can lead to the expression of egfp and hence appear green fluorescent. Since both effector binding to XylS and XylS binding to the Pm promoter are equilibrium reactions, egfp expression and thus fluorescence is dependent on the binding strength of the effector to XylS. Hence, determination of the fluorescent signal also indicates how strong an effector is bound to XylS. The use of a miniaturized cell-sorter can allow to select for fluorescent clones. Following the development of a screening method for XylS variants, binding of different effectors to the XylS wildtype protein is investigated and compared to published data.

Directed evolution of XylS. Similarly to above, inducible XylS variants are evolved by random mutagenesis of XylS and screening of the desired variants. Likely target effector molecules for XylS binding are chlorophenols, chlorobenzenes, phenol, benzenes, xylene, tolouene and cresols. Screening of a XylS variant library with several target effectors not only allows faster identification of desired variants, but also allows the assignment of effector binding arrays to each individual XylS variant. Hence, apart from identifying Xyls variants effectively binding a given effector and thus resulting in the expression of egfp even at low effector concentrations, an additional criteria for the regulation of designed pathways is a XylS regulator specifically activated by those substrates degraded by a particular pathway.

Construction of a strong, XylS inducible hybrid-promoter. Based on the information on XylS binding sequences within the Pm promoter sequence and the location of the −35 and −10 regions (Han, S., Eltis, L. D., Timmis, K. N., Muchmore, S. W. and Bolin, J. T. (1995) Science 270:976-980), a hybrid promoter containing the −35 and −10 regions of the stronger lac- or tac-promoter and the XylS binding region is constructed. A strong, XylS inducible promoter increases the expression of catabolic genes at low effector concentrations. Again, egfp expression is used to check XylS regulation and transcription levels of the constructed hybrid-promoter.

Expression of catabolic genes of designed pathways under the control of XylS regulation. The lac- or tac-promoter used for expression of catabolic genes during pathway design is replaced by the designed XylS inducible hybrid-promoter. Individual designed pathways assembled on pACmod and/or pFNmod and under the control of the hybrid promoter is transformed in E. coli. In order to achieve specific induction of the pathways, the respective evolved XylS variants, expressed under the control of the Ps2 promoter on a pUC-based plasmid, is co-transformed and the induction of catabolic gene expression determined.

### Combining Genes from Different Metabolic Pathways

The above section exemplifies directed evolution of genes combined from different biodegradation pathways. The same principles may also be applied to biosynthetic pathways. Many biologically active natural compounds contain additional modifications. The most common modification is the introduction of oxygen functions, which is of ten catalyzed by P450 monooxygenases. However, only a few such modifying enzymes have been cloned so far. The invention advantageously provides novel modifying enzymes resulting in novel or more efficiently produced modified compounds.

### Novel Cyclic Modified Terpenes

To introduce oxygen functions into terpene structures, the carotenoid monooxygenases (spheroidene monooxygenase crtA from Rhodobacter) can be used, as described above under the section entitled “Terpenoids”. Preliminary studies showed that these monooxygenases are evolvable to oxygenize different polyprene substrates. Different C20 (GGDP), C30 (squalene, dehydrosqualene) to C40 substrates can be created by evolving the carotenoid monooxygenases.

Terpene cyclases, like sesquiterpene cyclases, diterpene cyclases and triterpene cyclases (squalene-hopene cyclase and oxido-squalene cyclases) can then be adapted to the oxygenated polyprene substrates for the production of novel cyclic oxo-terpenes.

In addition, cyclic terpenoids containing oxygen functions may be further modified by evolving carotenoid glycosylating enzymes like zeaxanthin glucosylase crtX from Erwinia species to introduce carbohydrate functions into terpenoid structures.

### Bacterial P450 Monooxygenases

The bacterial monooxygenases P450 BM3 from Bacillus megaterium and P450CAM from Pseudomonas putida, which are both well expressed in recombinant microorganisms, may be useful for the oxidation of a variety of metabolites. These enzymes can be evolved to accept polyketide, carotenoid or terpenoid moieties as substrates and thus produce novel types of compounds.

### Epoxy-carotenoids

In addition, novel epoxy-carotenoids can be produced, e.g., by evolving the squalene epoxidase from S. cerevisiae to accept different acyclic carotenoids obtained in breeding experiments.

### EXAMPLES

The invention will be better understood by reference to the following Examples, which are provided by way of illustration and not limitation.

### Materials and Methods

Examples 1 and 2 employ the materials and methods described here.

Cloning and Culture Growth

Genes for GGDP synthase (crtE_{EU}), phytoene synthase (crtB_{EU}) phytoene desaturase (crtI_{EU}, crtI_{EH}) and lycopene desaturase (crtY_{EU}, crtY_{EH}) were amplified from genomic DNA of Erwinia uredovora (Pantoa ananatis DSM 30080) and Erwinia hericola EhoI (Pantoea ananatis DSM 30071) (GenBank accession codes:D90087, M87280, M99707) using a 5 ′PCR primer, which contained at its 5 ′end a XbaI-site (crtE_{EU}, crtB_{EU}, crtI_{EU}, crtI_{EH}) or a EcoRI-site (crtY_{EU}, crtY_{EH}) followed by the sequence 5′-AGG AGG ATT ACA AAA TG-3′ providing a shine-dalgarno sequence (underlined) and a start codon (bold), and a 3′PCR primer containing at its 5 ′end a EcoRI-site (crtE_{EU}, crtB_{EU}, crtI_{EU}, crtI_{EH}) or a NcoI-site (crtY_{EU}, crtY_{EH}). PCR products were then cloned into pUC19, which has been modified by deleting the lacZ-fragment and introducing a new multiple cloning site (5′-XbaI-SmaI-EcoRI-NcoI-NotI), thereby changing the operator sequence to facilitate constitutive expression. GGDP-synthase (crtE_{EU}) and phytoene desaturase (crtB_{EU}) were sugcloned into the BamHI-site (crtB_{EU}) or ClaI-site (crtE_{EU}) of pACmod (pACYC184 devoid of the XbaI-site) by amplification of the genes together with the lac-promoter using primer which introduce at both sites a BamHI-site or ClaI-site, respectively. The two reading frames face each other in the resulting plasmid pAC-crtE_{EU}-crtB_{EU}. Similarly, phytoene desaturase (wildtype or mutant) was subcloned from pUC into the HindIII site of pAC-crtE_{EU}-crtB_{EU} to give pAC-crtE_{EU}-crtB_{EU}-crtI_{EU}/crtI_{EH}/I14 where both genes crtE_{EU} and phytoene desaturase have the same orientation. For carotenoid biosynthesis, transformed E. coli JM101 or the recombination deficient strain JM109 (for stable propagation of mutant I14 during carotenoid biosynthesis) were cultivated for 24 hrs at 28° C. in the dark in LB-medium (500 ml medium in 1l flask) supplemented with 50 μg ml⁻¹chloramphenicol and 50 μg ml⁻¹ carbenicillin.

Analysis of Carotenoids

Wet cells (0.3 mg) were extracted with 1 ml acetone and reextracted with an equal volume of hexane after addition of ⅕ volume water. 20 μl of extract was applied to a Spherisorb ODS 2 column (250×4.6 mm, 5 μm, Waters), and eluted with acetonitrile: isopropanol (99:1) at a flow-rate of 2 ml/min using an Alliance HPLC system equipped with a photodiode array detector from Waters. Mass spectra were obtained with a Hewlett-Packard (Agilent Technologies, Palo Alto, Calif.) Series 1100 LC/MSD coupled with APCI (atmosphere pressure chemical ionization) interface.

DNA-Shuffling and Library Screening

A library of phytoene desaturase variants was created by DNA shuffling of the genes crtI_{EU} and crtI_{EH} from Erwinia uredovora and Erwinia herbicola, respectively, using the protocol from Stemmer (Stemmer, W. P. C., Nature, 1994, 370:389-391). The final amplification products were ligated into pUC and transformed into phytoene-producing E. coli JM101 cells containing pAC-crtE_{EU}-crtB_{EU} Transformants were plated on LB plates supplemented with 50 μg ml⁻¹ carbenicillin and chloramphenicol. After 24 hrs of incubation at 30° C. in the dark, colonies were replicated using a nitrocellulose membrane and transferred onto fresh LB plates. Colonies were screened visually for color variants after an additional 12 hrs (or until color developed) incubation. Overnight cultures (5 ml LB) were inoculated with selected colonies for analysis of carotenoid synthesis. A library of lycopene cyclase variants was created by shuffling crtY_{EU} and crtY_{EH} from Erwinia uredovora and Erwinia hericola, respectively. After ligation into pUC, the library was used to transform E. coli JM109 cells harboring plasmid pAC-crtE_{EU}-crtB_{EU}-I14.

### EXAMPLE 1

Biosynthesis of New Carotenoids in E. coli

This example describes shuffling two genes encoding phytoene desaturases within a cartenoid biosynthetic pathway assembled from genes isolated from different bacterial species and screening the resulting library for novel carotenoids. One desaturase chimera introduced six rather than four double bonds into phytoene, allowing the pathway to produce the fully-conjugated carotenoid, 3,4,3′,4′-tetradehydrolycopene.

To enable biosynthesis of new carotenoids in E. coli, the phytoene desaturase (crtI) and the lycopene cyclase (crtY) for in vitro evolution was targeted. These enzymes are located at important branch points of the carotenoid biosynthetic pathway and determine the types of acyclic or cyclic carotenoids produced (see, FIG. 1). The first goal was to convert the four-step desaturase from Erwinia into an efficient six-step desaturase, in order to synthesize the strong antioxidant, 3,4,3′,′-tetradehydrolycopene in E. coli.

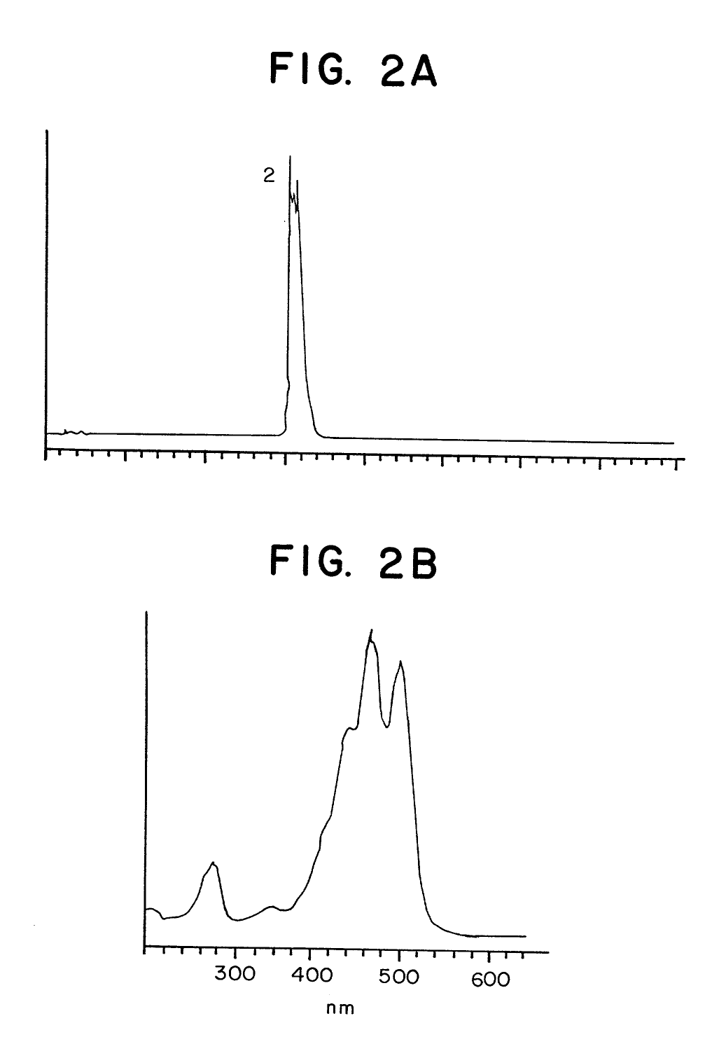

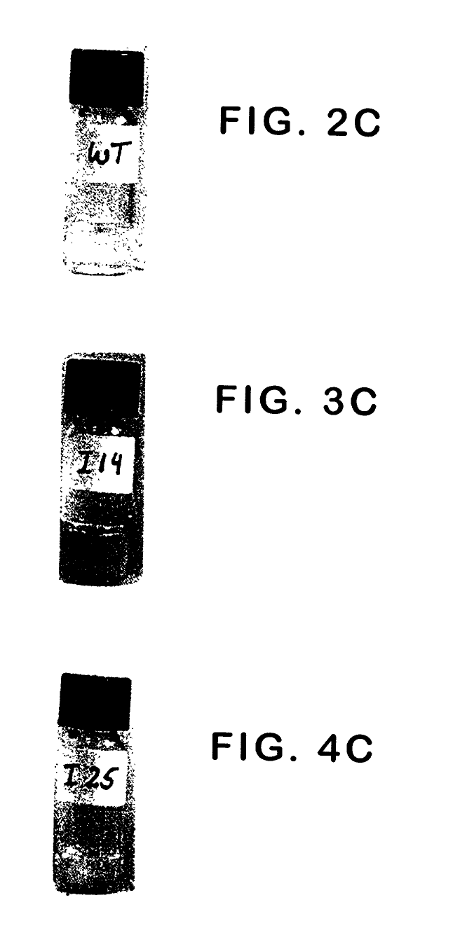

E. coli cells co-transformed with pAC-crtE_{EU}-crtB_{EU}, expressing the GGDP synthase (crtB_{EU}) and the phytoene synthase (crtE_{EU}) from Erwinia uredovora (EU), and with pUC-crtI_{EU} or pUC-crtI_{EH} expressing the phytoene desaturases (crtI) from E. uredovora and E. herbicola (EH), respectively, produced lycopene as the exclusive carotenoid as determiend by HPLC analysis (FIG. 2A) and the absorption spectrum of the peak (FIG. 2B). These cells appeared orange to orange-red on plates and in liquid culture (FIG. 2C).

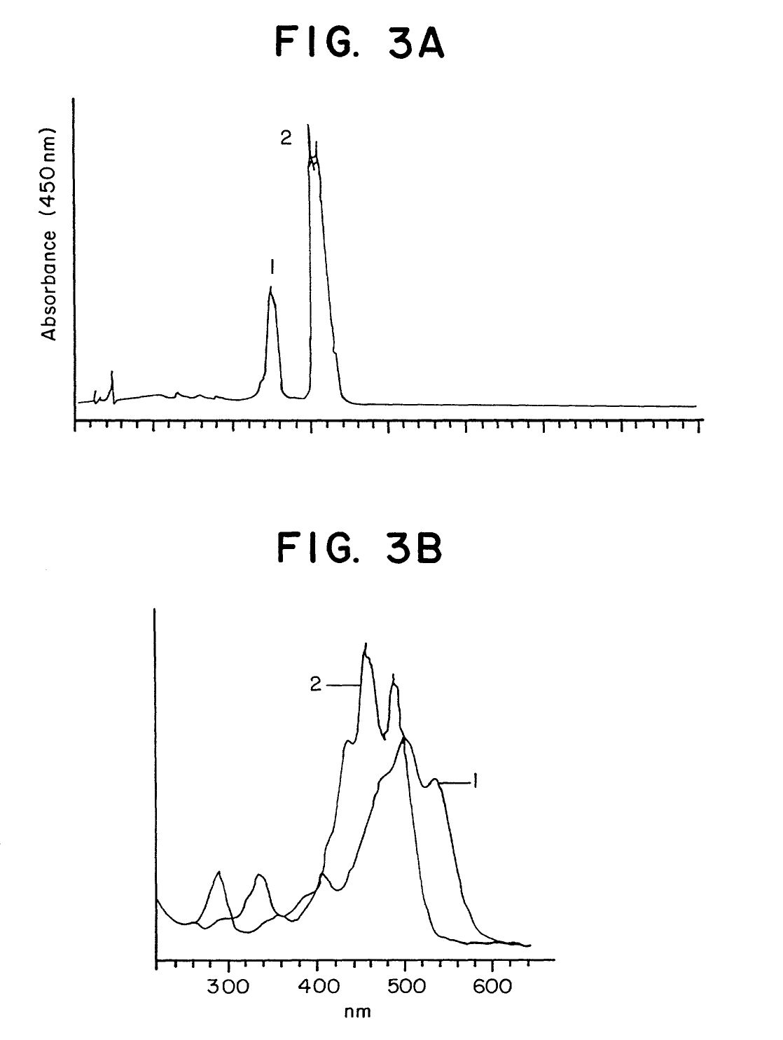

A library of desaturases generated by in vitro homologous recombination (DNA shuffling; Stemmer, W. P. C., Nature, 1994, 370:389-391) of the genes from E. herbicola and E. uredevoa was transformed into phytoene-synthesizing E. coli JM 101 harboring pAC-crtE_{EU}-crtB_{EU}. Colonies were transferred to nitrocellulose membranes, which provide a white background for visual screening of the clones based on color. Approximately 10,000 colonies were screened; 30% appeared white due to inactivation of the desaturase. Twenty colonies were yellow, indicating the presence of carotenoids with fewer conjugated double bonds than lycopene. In addition, one pink clone (I14) (FIG. 3C) was identified, suggesting the introduction of additional double bonds into lycopene by this mutant.

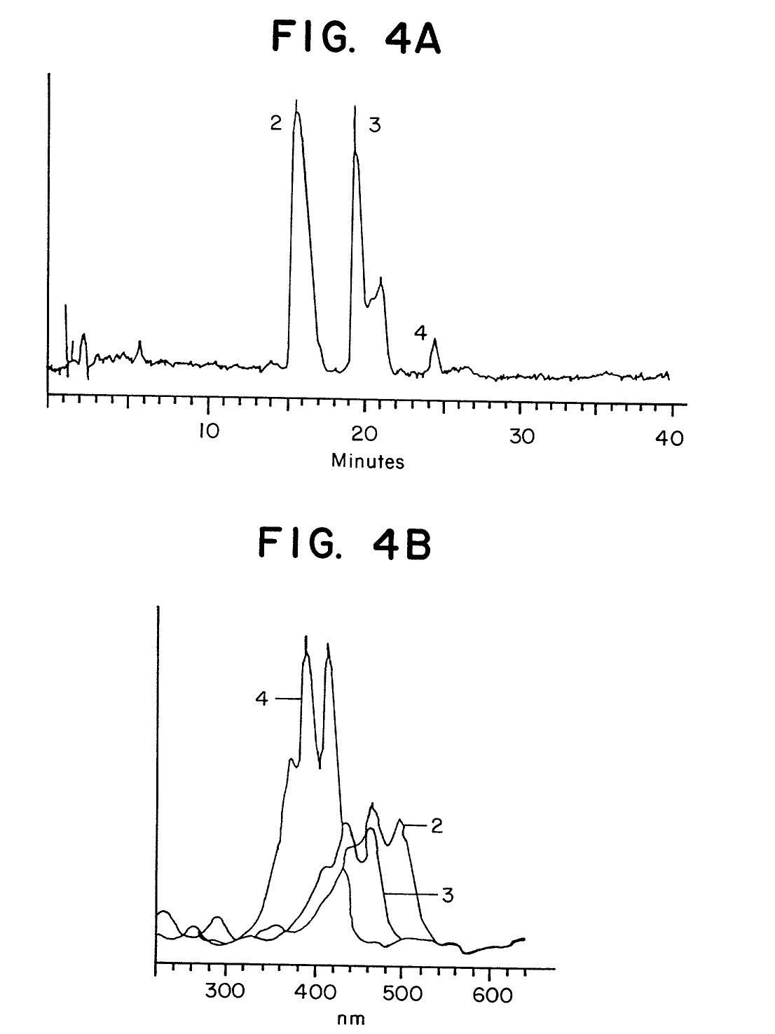

The carotenoid extracts of cells from one yellow clone (I25) (FIG. 4C), I14 and wildtype were analyzed by HPLC (FIGS. 2A, 3A, and 4A). The following carotenoids were identified: peak 1:3,4,3′4′-tetradehydrolycopene (λₘₐₓnm:480 510 540), peak 2: lycopene (λₘₐₓnm:444 470 502), Peak 3: neurosporene (λₘₐₓnm:415 440 468), peak 4: ξ-carotene (λₘₐₓnm:378 400 425). Double peaks indicate different geometrical isomers. Absorption spectra showed for the main products absorption maxima typical for ξ-carotene, 3,4,3,4-tetradehydrolycopene and lycopene, respectively (Britton et al., supra, 1995). Further analysis by high pressure liquid chromatography (HPLC) shows that the desaturase of mutant I14 introduces two double bonds in lycopene, which leads to the accumulation of 3,4,3′,4′-tetradehydrolycopene in addition to lycopene (FIG. 3B). Mutant I25 catalyzes the introduction of two double bonds in phytoene. Reflecting the stepwise nature of desaturation, mutant I25 synthesizes neurosporene and lycopene, in addition to the main product, ξ-carotene (FIGS. 4A and 4B).

Sequence analysis of the I25 desaturase showed two amino acid changes, R332H and G470S, in the sequence of crtI_{EU} and no recombination. G470S is located in a hydrophobic C-terminal domain that is thought to be involved in substrate binding and the dehydrogenation reaction and is conserved among carotenoid desaturases (Armstrong et al., supra, 1989). In mutant I14, the N-terminus (residues 1-39) of the desaturase from E. uredovora is replaced with that of E. herbicola, which differs in only four residues (P3K, T5V, V27T, L28V). The I14 desaturase also contained two amino acid substitutions, F291L and A269V.

Two chimeras were constructed to determine whether the N-terminal recombination or the point mutations (or both) were responsible for the altered catalytic activity of mutant I14. Chimera I contained only the recombined N-terminus, and chimera II contained only the two amino acid changes. Only chimera I exhibited the altered catalytic activity of mutant I14. The N-terminus comprises a typical dinucleotide binding domain (Gly-Xaa-Gly-(Xaa)₂-Ala/Gly-(Xaa)₃-Ala-(Xaa)₆-Gly) (Wierenga et al., J. Mol. Biol., 1986, 187:101-107) not previously associated with substrate specificity. Co-factor binding (FAD in Erwinia desaturases; Fraser et al., supra, 1992) might play an important role in controlling desaturation.

### EXAMPLE 2

### Biosynthesis of Cyclic Carotenoids in E. coli

The pathway described in Example 1 was extended with a library of genes encoding shuffled lycopene cyclases. This example describes the attempt to produce new pathways for the biosynthesis of cyclic carotenoids by in vitro evolution of the cyclase (FIG. 1). This experiment was based on the hypothesis that wildtype lycopene cyclase or a closely-related variant might also cyclize 3,4-didehydrolycopene. From this new set of pathways, one produces, for the first time, the cyclic carotenoid torulene in a bacteria ( E. coli).

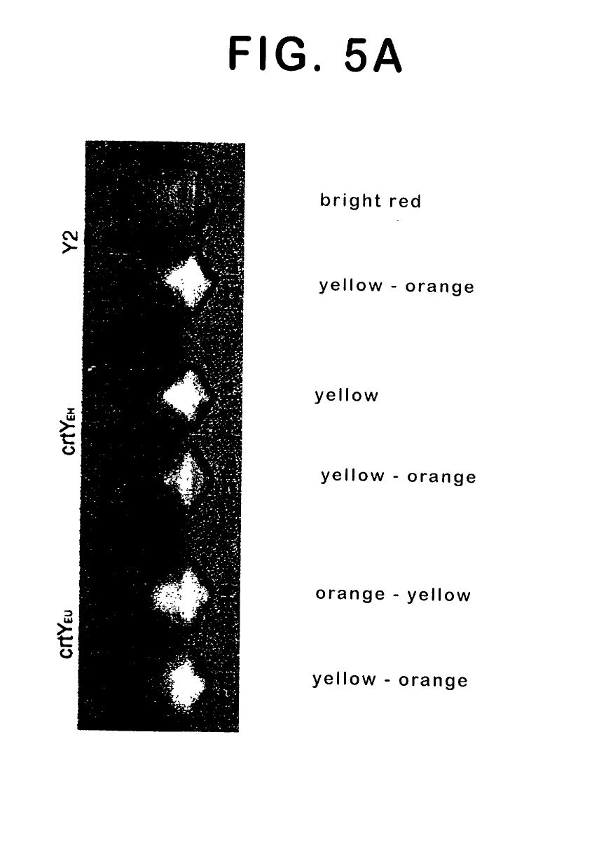

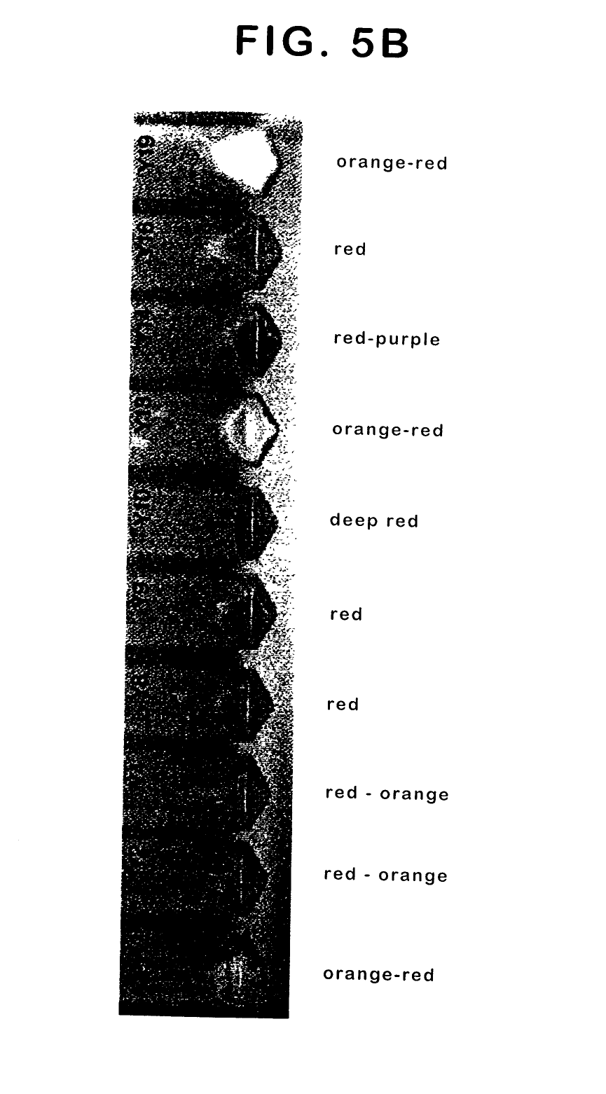

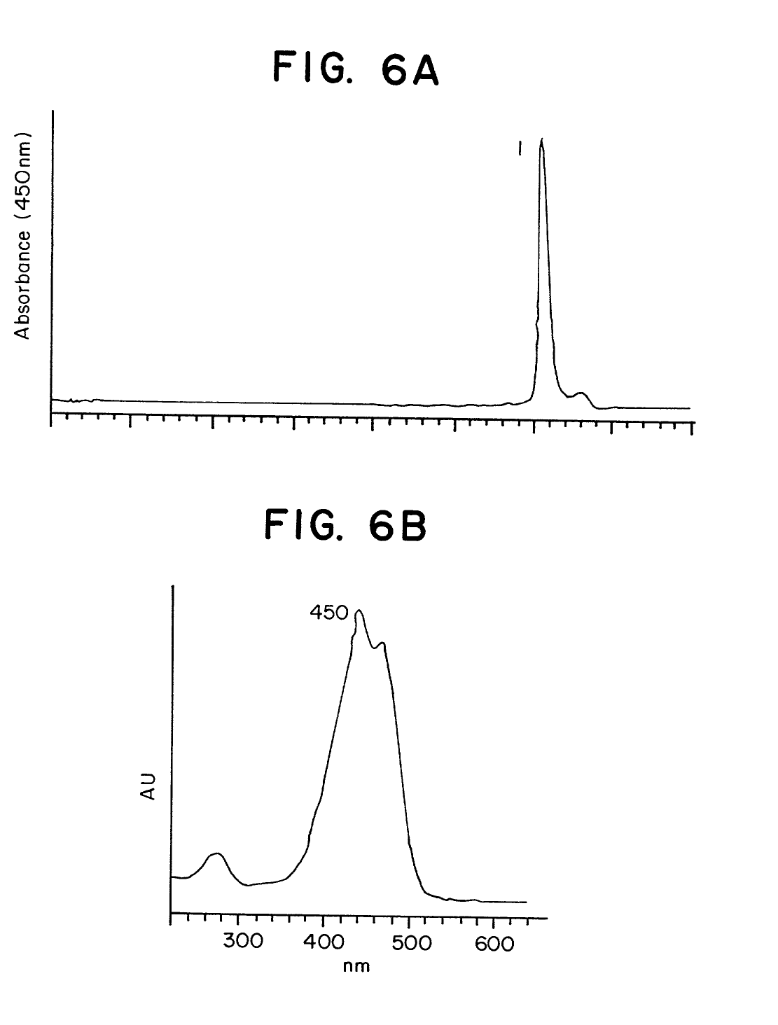

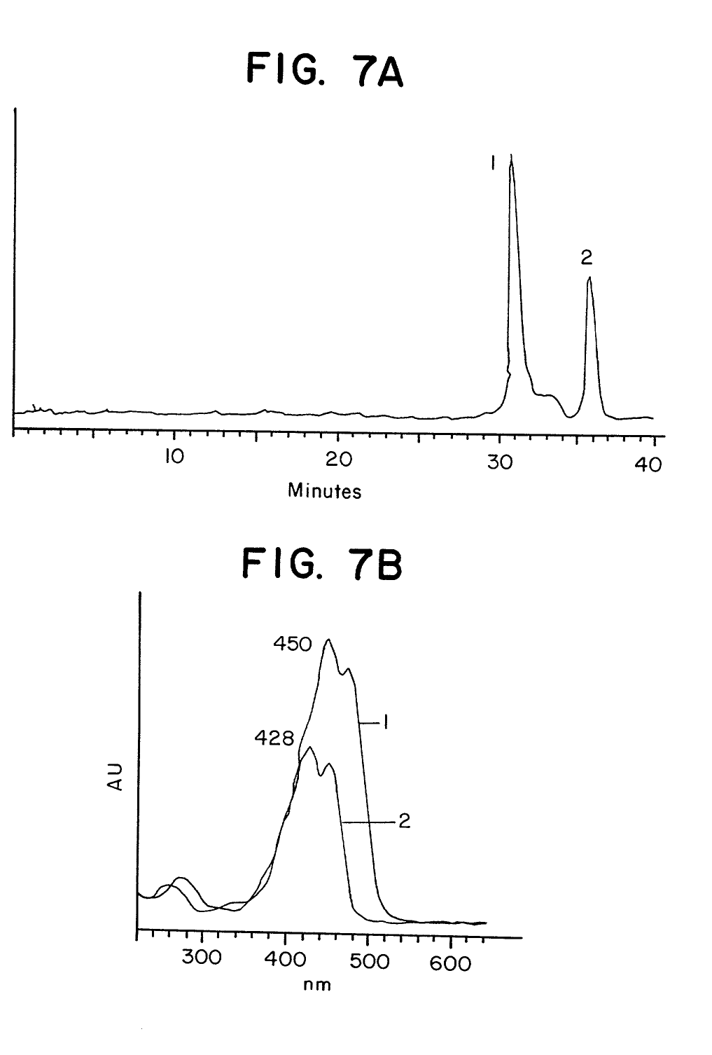

The biosynthetic pathway consisting of GGDP synthase (crtB_{EU}), phytoene synthase (crtE_{EU}) and either wildtype phytoene desaturase (crtI_{EU}) or mutant I14 was extended with the genes for the lycopene cyclase (crtY) from E. uredovora or E. herbicola by cloning the desaturase genes into pAC-crtE_{EU}-crtB_{EU} to yield pAC-crtE_{EU}-crtB_{EU}-crtI_{EU}/I14 and complementation of E. coli pAC-crtE_{EU}-crtB_{EU}-crtI_{EU} /I14 with pUC-crtY_{EU} or pUC-crtY_{EH}. E. coli cells expressing wildtype desaturase crtI_{EU} on pAC-crtE_{EU}-crtB_{EU}-crtI_{EU} together with the lycopene cyclases crtY_{EU} or crtY_{EH} on pUC-crtY_{EU} or pUC-crtY_{EH}, respectively, synthesized predominantly β, β-carotene from lycopene and turned bright yellow-orange (FIG. 5A). A less-polar carotenoid with a spectrum typical for β-zeacarotene, the monocyclic product derived from neurosporene, is also produced (FIGS. 7A and 7B). In contrast, E. coli expressing I14 desaturase together with the wildtype lycopene cyclases only synthesized β,β-carotene (FIGS. 6A and 6B) and developed a bright orange color (FIG. 5A). Neither 3,4,3′,4′-tetradehydrolycopene nor its cyclization products are synthesized in E. coli pAC-crtE_{EU}-crtB_{EU}-I14 expressing wildtype lycopene cyclases, suggesting that lycopene (the precursor to 3,4,3′,4′-tetradehydrolycopene) is a good substrate for the cyclases. Desaturase variant I14 appears to have higher desaturation activity than the wildtype enzyme, since no neurosporene accumulates that can be cyclized to β-zeacarotene.

A library of lycopene cyclases was created by shuffling the genes crtY_{EU} and crtY_{EH}. This library was used to transform E. coli cells harboring pAC-crtE_{EU}-crtB_{EU}-I14 encoding the extended desaturation pathway. Among approximately 4,500 clones screened, 20% were pink due to inactivation of the cyclase. Twenty-five colonies that were orange-red to purple-red, indicating the possible cyclization of 3,4-didehydrolycopene, were selected. The selected clones exhibited a variety of colors (FIG. 5B) and accumulated different ratios of lycopene, 3,4, 3′4′-tetradehydrolycopene and β,β-carotene (clones expressing wildtype enzymes formed only β,β-carotene).

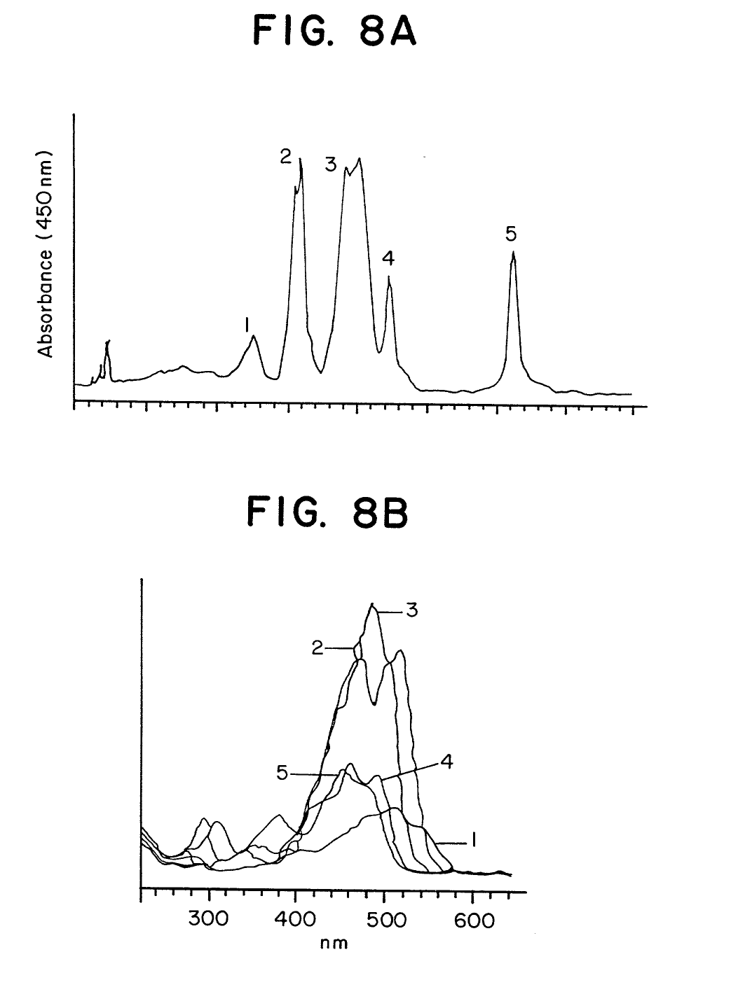

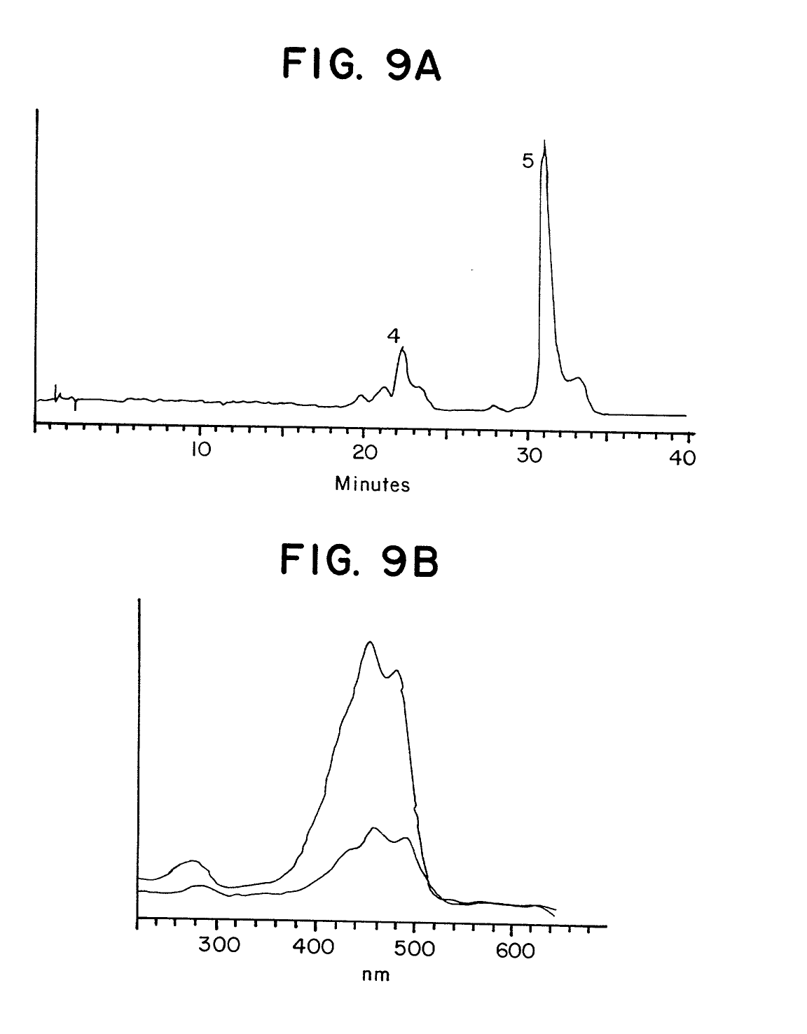

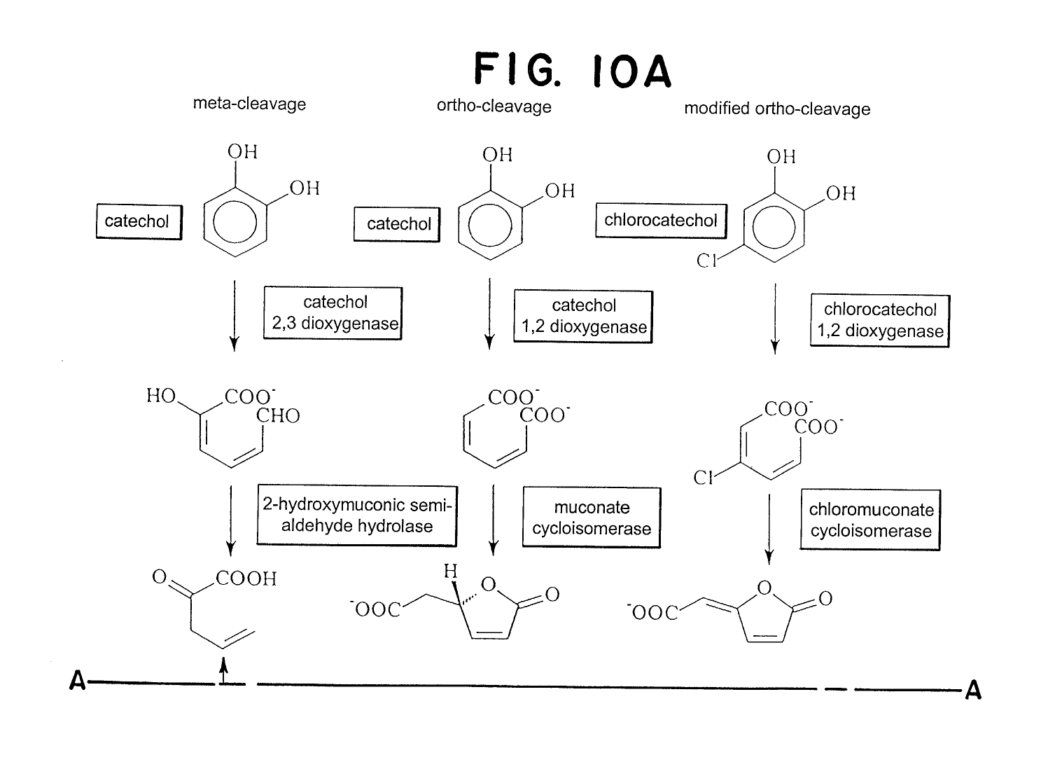

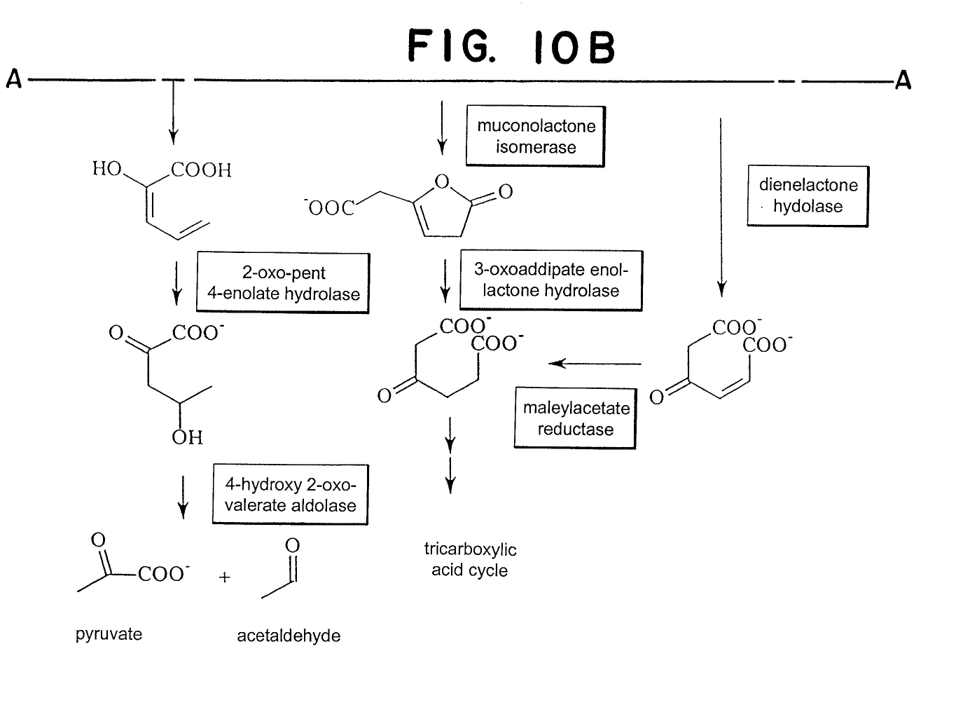

Clone Y2 appeared bright red compared to the yellow-orangecolor of the wildtype (FIG. 5A); its extract showed a marked absorption maximum of 480 nm. HPLC analysis revealed not only the acyclic carotenoids lycopene and 3,4,3′,4′-tetradehydrolycopene, but also the cyclization products of lycopene, β,β-carotene and β,Ψ-carotene, as well as a new, major carotenoid (FIG. 8A). The absorption maxima (Britton et al., supra, 1995), mass and polarity of this new product correspond to those of torulene, the cyclization product of 3,4-didehydrolycopene (FIG. 1). When the cyclase from mutant Y2 was expressed with the wildtype desaturase, the bacteria synthesized monocyclic β,Ψ-carotene and dicyclic β,β-carotene from lycopene, but no torulene (FIG. 9A).

Torulene has been identified in red yeasts such as Rhodotorula and Phaffia (Johnson and Schroeder, Adv. Biochem. Eng. Biotechnol., 1995, 53:119-178). However, analysis of pigment accumulation in Rhodotorula glutinis and Phaffia rhodozyma suggested biosynthesis of torulene from β-zeacarotene, the monocyclic product derived from neurosporene, through desaturation of the 7,8-dihydro-Ψ end group rather than cyclization of 3,4-didehydrolycopene (Britton, supra, 1998; An et al., J. Biosci. Bioeng., 1999, 88:189-193). The enzyme catalyzing this desaturation has not yet been characterized. Sequence analysis of mutant Y2 revealed two amino acid changes, R330H and P367S, in the sequence of the E. uredovora cylcase and no recombination. Neither mutation is located in motifs conserved among various cyclases (Cunningham et al., Plant Cell, 1996, 8:1613-1626).

Extension of the pathway to 3,4-didehydrolycopene with a functional cyclase was accomplished by DNA shuffling, leading to the first reported synthesis of torulene in E. coli. Torulene is also not produced by the organisms from which the biosynthetic genes were obtained. Furthermore, torulene production in yeasts follows a different synthetic strategy. Thus the in vitro evolution has extended the biosynthetic pathway with a catalytic function currently not available from a natural source. Assembling biosynthetic genes into a pathway and evolving key enzymes is an efficient strategy for the synthesis of new metabolites in E. coli. In vitro evolution allowed us to engineer the catalytic properties of two enzymes for which there is no three-dimensional structure and little knowledge of the catalytic mechanism. Addition of new biosynthetic genes and further evolution should allow us to produce yet more novel carotenoids in E. coli.

These approaches of rational pathway assembly and directed evolution allow the discovery and production of many new compounds that are for all practical purposes inaccessible from natural sources or by synthetic chemistry.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all disclosed or experimentally determined values are approximate, and are provided for description only.

All patents, patent applications, publications, experimental protocols, and other materials cited herein are hereby incorporated herein reference in their entireties.

What is claimed is:

1. A library of host cells, wherein each host cell comprises an expression vector that expresses a mutated gene encoding a biometabolic enzyme operably associated with an expression control sequence, the enzyme being one component of a biometabolic pathway, and wherein
(a) the mutated gene is a chimera of genes from different metabolic pathways; or
(b) the enzyme is isolated from a biometabolic pathway different from the biometabolic pathway of which it is a component in the host cell; or
(c) the biometabolic pathway is a carotenoid biosynthetic pathway.

2. The library of claim 1, wherein a host cell further comprises a second mutated gene encoding a biometabolic enzyme.

3. The library of claim 1, wherein the host cells are bacterial host cells.

4. The library of claim 3, which E. coli host cells express genes necessary for the production of starting materials for the biometabolic pathway.

5. The library of claim 1, wherein the biometabolic pathway is a biosynthesis pathway for a class of compounds selected from terpenoids, carotenoids, polyketides, flavonoids, tetrapyrroles, aminoglycosides, and non-ribosomally produced polypeptides.

6. The library of claim 1, wherein the biometabolic pathway is a biodegradation pathway.

7. The library of claim 1, wherein the biometabolic enzyme is a component of a biosynthesis pathway for a class of compounds selected from terpenoids, carotenoids, polyketides, flavonoids, tetrapyrroles, aminoglycosides, and non-ribosomally produced polypeptides

8. The library of claim 7 wherein the carotenoid biosynthesis enzyme is selected from the group consisting of GGDP synthase, a phytoene synthase, a phytoene desaturase, a lycopene β-cyclase, a lycopene ε-cyclase, a spheroidene monoxygenase, a β-carotene oxygenase, a methoxyneurosporene desaturase, a zeanthin glucosylase, a β-carotene hydroxylase, and a β-carotene desaturase, a dehydrosqualene synthase, and a dehydrosqualene desaturase.

9. The library of claim 1, wherein the biometabolic enzyme is a component of a biodegradation pathway.

10. The library of claim 1, wherein the mutated gene is a chimera of two homologous genes derived from different species.

11. The library of claim 1, wherein the mutated gene is a chimera derived from homologous genes from different biometabolic pathways.

12. A host cell which produces a novel biosynthetic product, which host cell is selected from the library of claim 1.

13. A method for producing a biometabolic product, which method comprises culturing a host cell comprising an expression vector that expresses a mutated biometabolic gene operably associated with an expression control sequence, under conditions that permit production of the product by the host cell, wherein the host cell is selected from the library in claim 1.

14. The method according to claim 13, wherein the host cell further comprises a second mutated biometabolic gene.

15. The method according to claim 13, wherein the host cell is a bacterial host cell.

16. The method according to claim 15, wherein the host cell is an E. coli, which E. coli expresses genes necessary for the production of starting materials for the biometabolic pathway.

17. The method according to claim 13, wherein the biometabolic product is a carotenoid and the mutated gene encodes for a carotenoid biosynthesis enzyme, selected from the group consisting of a GGDP synthase, a phytoene synthase, a phytoene desaturase, a lycopene β-cyclase, a lycopene ε-cyclase, a spheroidene monoxygenase, a β-carotene oxygenase, a methoxyneurosporene desaturase, azeanthin glucosylase, a β-carotene hydroxylase, and a β-carotene desaturase, a dehydrosqualene synthase, and a dehydrosqualene desaturase.

18. The method according to claim 17, wherein the carotenoid is a novel carotenoid.

19. The method according to claim 13, wherein the mutated gene encodes for a biosynthesis enzyme which is a component of a biosynthesis pathway for a class of compounds selected from terpenoids, polyketides, flavonoids, tetrapyrroles, aminoglycosides, and non-ribosomally produced polypeptides

20. A method for creating a new biometabolic pathway, which method comprises detecting production of a biometabolic compound in a host cell modified by transduction with a mutated gene encoding abiometabolic enzyme, wherein the biometabolic compound is not produced by the host cell in the absence of the modification, wherein
(a) the mutated gene is a chimera of genes from different metabolic pathways; or
(b) the enzyme is isolated from a metabolic pathway different from the biometabolic pathway of which it is a component in the host cell; or
(c) the biometabolic pathway is a carotenoid biosynthetic pathway.

21. The method according to claim 20, wherein the biometabolic enzyme is a carotenoid biosynthesis enzyme selected from the group consisting of a GGDP synthase, a phytoene synthase, a phytoene desaturase, a lycopene β-cyclase, a lycopene ε-cyclase, a spheroidene monoxygenase, a β-carotene oxygenase, a methoxyneurosporene desaturase, a zeanthin glucosylase, a β-carotene hydroxylase, a β-carotene desaturase, a dehydrosqualene synthase, and a dehydrosqualene desaturase.

22. The method according to claim 21, wherein the carotenoid biosynthesis enzyme is selected from the group consisting of crtI from Erwinia hericola, crtI from Erwinia uredovora, crtY from Erwinia hericola, and crtY from Erwinia uredovora.

23. A nucleic acid encoding a phytoene desaturase selected from the group consisting of (i) an E. uredovora crtI comprising an arginine to histidine modification at position 332 and a glysine to serine substitution at position 470, and (ii) a E. uredovora crtI comprising a proline to lysine modification at position 3, a threonine to valine modification at position 5, a valine to threonine modification at position 27, and a leucine to valine modification at position 28.

24. An expression vector comprising the nucleic acid of claim 23 operably associated with an expression control sequence.

25. A host cell comprising the expression vector of claim 24.

26. A nucleic acid encoding a lycopene cyclase (crtY) from E. uredovora comprising an arginine to histidine modification at position 330 and a proline to serine modification at position 367.

27. An expression vector comprising the nucleic acid of claim 26 operably associated with an expression control sequence.

28. A host cell comprising the expression vector of claim 27.

29. An expression vector comprising a sequence for a mutated gene encoding a biometabolic enzyme operably associated with an expression control sequence, the enzyme being one component of a metabolic pathway, and wherein
(a) the mutated gene is a chimera of genes from different metabolic pathways; or
(b) the enzyme is isolated from a biometabolic pathway different from the biometabolic pathway of which it is a component in the host cell; or
(c) the biometabolic pathway is a carotenoid biosynthetic pathway.

30. The expression vector of claim 29, wherein the biometabolic enzyme is a component of a biosynthesis pathway for a class of compounds selected from terpenoids, carotenoids, polyketides, flavonoids, tetrapyrroles, amino glyco sides, and non-ribosomally produced polypeptides

31. The expression vector of claim 29, wherein the biometabolic enzyme is a component of a biodegradation pathway.
